(19) United States
(12) Patent Application Publication
(10) Pub. No.: US 2022/0061746 A1
(43) Pub. Date: Mar. 3, 2022

(54) RISK ASSESSMENT SYSTEM AND METHODS FOR USE THEREWITH

(71) Applicant: Enlitic, Inc., San Francisco, CA (US)

(72) Inventors: Kevin Lyman, Fords, NJ (US); Ben Covington, JR., Berkeley, CA (US); Anthony Upton, Malvern (AU)

(73) Assignee: Enlitic, Inc., San Francisco, CA (US)

(21) Appl. No.: 17/007,924

(22) Filed: Aug. 31, 2020

(51) Int. Cl. A61B 5/00; G16H 50/30; G16H 70/20; G16H 50/70; G16H 40/20; G16H 70/60; G16H 30/20; G16H 30/40; G16H 50/20; G16H 15/00

(52) U.S. Cl. CPC A61B 5/411; A61B 5/4519; G16H 70/20; G16H 50/70; G16H 40/20; G16H 70/60; G16H 30/20; G16H 30/40; G16H 50/20; G16H 15/00; A61B 5/7275; A61B 5/0022; A61B 5/7267; A61B 5/742; A61B 5/4528; A61B 5/4872; G16H 50/30

(57) ABSTRACT

A risk assessment system is configured to receive patient history data for a patient. A set of risk assessment scores corresponding to the patient are generated for a set of risk assessment categories based on applying at least one risk assessment function to the patient history data. One of the set of risk assessment categories is identified as high risk for the patient based on a corresponding one of the set of risk assessment scores. A high risk protocol corresponding to the one of the set of risk assessment categories is identified, and performance of the high risk protocol is facilitated for the patient based on identification of the one of the set of risk assessment categories as high risk for the patient.

### CROSS-REFERENCE TO RELATED APPLICATIONS

None

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISC

Not applicable.

### BACKGROUND

### Technical Field

This invention relates generally to medical imaging devices and knowledge-based systems used in conjunction with client/server network architectures.

### Description of Related Art

[symbol]

### BRIEF DESCRIPTION OF THE SEVERAL VIEWS OF THE DRAWING(S)

FIG. 1 is a schematic block diagram of an embodiment of a medical scan processing system;

FIG. 2A is a schematic block diagram of a client device in accordance with various embodiments;

FIG. 2B is a schematic block diagram of one or more subsystems in accordance with various embodiments;

FIG. 3 is a schematic block diagram of a database storage system in accordance with various embodiments;

FIG. 4A is schematic block diagram of a medical scan entry in accordance with various embodiments;

FIG. 4B is a schematic block diagram of abnormality data in accordance with various embodiments;

FIG. 5A is a schematic block diagram of a user profile entry in accordance with various embodiments;

FIG. 5B is a schematic block diagram of a medical scan analysis function entry in accordance with various embodiments;

FIGS. 6A-6B are schematic block diagram of a medical scan diagnosing system in accordance with various embodiments;

FIG. 7A is a flowchart representation of an inference step in accordance with various embodiments;

FIG. 7B is a flowchart representation of a detection step in accordance with various embodiments;

FIGS. 8A-8F are schematic block diagrams of a medical picture archive integration system in accordance with various embodiments;

FIG. 9 is a flowchart representation of a method for execution by a medical picture archive integration system in accordance with various embodiments;

FIG. 10A is a schematic block diagram of a de-identification system in accordance with various embodiments;

FIG. 10B is an illustration of an example of anonymizing patient identifiers in image data of a medical scan in accordance with various embodiments;

FIG. 11 presents a flowchart illustrating a method for execution by a de-identification system in accordance with various embodiments;

FIG. 12A is a schematic block diagram of a risk assessment system in accordance with various embodiments;

FIG. 12B is a schematic block diagram illustrating performance of a set of risk assessment functions in accordance with various embodiments;

FIGS. 12C and 12D illustrate example entries of a risk assessment database in accordance with various embodiments;

FIGS. 12F-12G are schematic block diagrams of a risk assessment system in accordance with various embodiments;

FIG. 12H illustrates example entries of a risk assessment database in accordance with various embodiments;

FIGS. 12I-12J are schematic block diagrams of a risk assessment system in accordance with various embodiments;

FIG. 12K presents a flowchart illustrating a method in accordance with various embodiments;

FIG. 13A is a schematic block diagrams of a scan review alert system in accordance with various embodiments; and

FIG. 13B presents a flowchart illustrating a method in accordance with various embodiments.

### DETAILED DESCRIPTION

The present U.S. Utility patent application is related to U.S. Utility application Ser. No. 15/627,644, entitled “MEDICAL SCAN ASSISTED REVIEW SYSTEM”, filed 20 Jun. 2017, which claims priority pursuant to 35 U.S.C. § 119(e) to U.S. Provisional Application No. 62/511,150, entitled “MEDICAL SCAN ASSISTED REVIEW SYSTEM AND METHODS”, filed 25 May 2017 and is also related to U.S. Utility application Ser. No. 16/353,935, entitled “LESION TRACKING SYSTEM”, filed on 14 Mar. 2019, which claims priority pursuant to 35 U.S.C. § 119(e) to U.S. Provisional Application No. 62/770,334, entitled “LESION TRACKING SYSTEM”, filed on 21 Nov. 2018, all of which are hereby incorporated herein by reference in their entirety and made part of the present U.S. Utility patent application for all purposes.

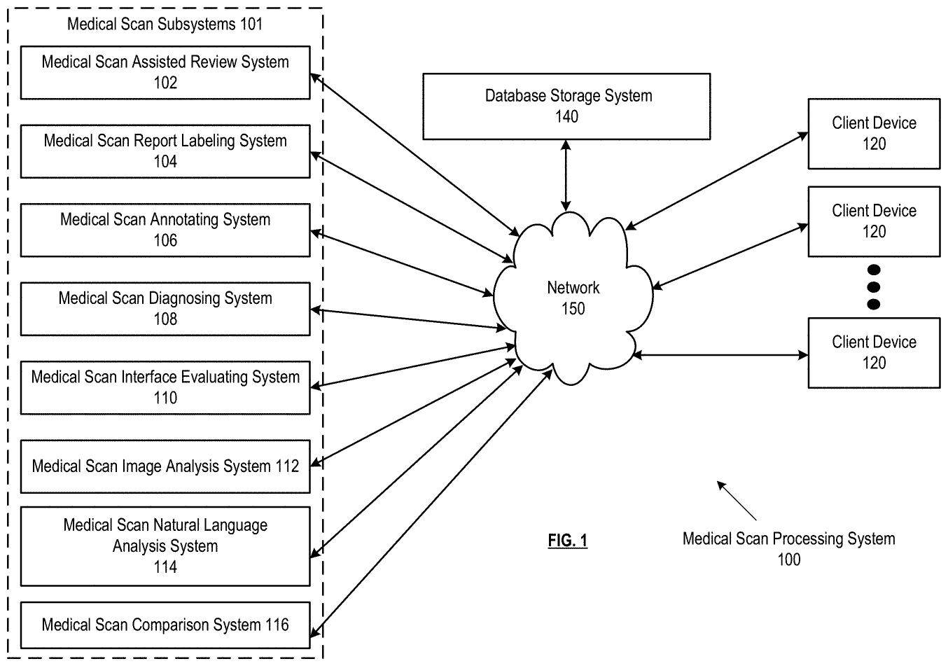

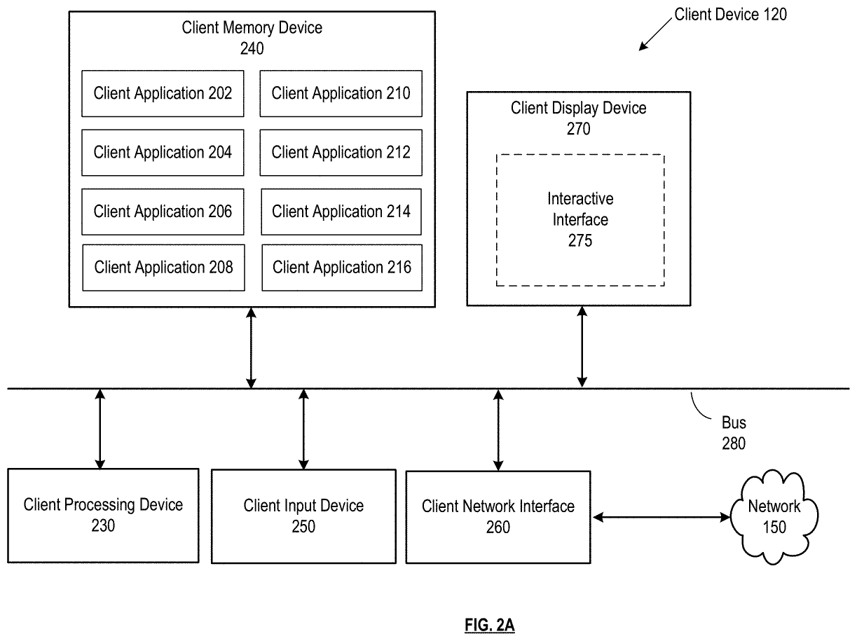

FIG. 1 presents a medical scan processing system 100, which can include one or more medical scan subsystems 101 that communicate bidirectionally with one or more client devices 120 via a wired and/or wireless network 150. The medical scan subsystems 101 can include a medical scan assisted review system 102, medical scan report labeling system 104, a medical scan annotator system 106, a medical scan diagnosing system 108, a medical scan interface feature evaluator system 110, a medical scan image analysis system 112, a medical scan natural language analysis system 114, and/or a medical scan comparison system 116. Some or all of the subsystems 101 can utilize the same processing devices, memory devices, and/or network interfaces, for example, running on a same set of shared servers connected to network 150. Alternatively or in addition, some or all of the subsystems 101 be assigned their own processing devices, memory devices, and/or network interfaces, for example, running separately on different sets of servers connected to network 150. Some or all of the subsystems 101 can interact directly with each other, for example, where one subsystem's output is transmitted directly as input to another subsystem via network 150. Network 150 can include one or more wireless and/or wired communication systems; one or more non-public intranet systems and/or public internet systems; and/or one or more local area networks (LAN) and/or wide area networks (WAN).

The medical scan processing system 100 can further include a database storage system 140, which can include one or more servers, one or more memory devices of one or more subsystems 101, and/or one or more other memory devices connected to network 150. The database storage system 140 can store one or more shared databases and/or one or more files stored on one or more memory devices that include database entries as described herein. The shared databases and/or files can each be utilized by some or all of the subsystems of the medical scan processing system, allowing some or all of the subsystems and/or client devices to retrieve, edit, add, or delete entries to the one or more databases and/or files.

The one or more client devices 120 can each be associated with one or more users of one or more subsystems of the medical scan processing system. Some or all of the client devices can be associated with hospitals or other medical institutions and/or associated with medical professionals, employees, or other individual users for example, located at one or more of the medical institutions. Some of the client devices 120 can correspond to one or more administrators of one or more subsystems of the medical scan processing system, allowing administrators to manage, supervise, or override functions of one or more subsystems for which they are responsible.

Some or all of the subsystems 101 of the medical scan processing system 100 can include a server that presents a website for operation via a browser of client devices 120. Alternatively or in addition, each client device can store application data corresponding to some or all subsystems, for example, a subset of the subsystems that are relevant to the user in a memory of the client device, and a processor of the client device can display the interactive interface based on instructions in the interface data stored in memory. For example, the website presented by a subsystem can operate via the application. Some or all of the web sites presented can correspond to multiple subsystems, for example, where the multiple subsystems share the server presenting the website. Furthermore, the network 150 can be configured for secure and/or authenticated communications between the medical scan subsystems 101, the client devices 120 and the database storage system 140 to protect the data stored in the database storage system and the data communicated between the medical scan subsystems 101, the client devices 120 and the database storage system 140 from unauthorized access.

The medical scan assisted review system 102 can be used to aid medical professionals or other users in diagnosing, triaging, classifying, ranking, and/or otherwise reviewing medical scans by presenting a medical scan for review by a user by transmitting medical scan data of a selected medical scan and/or interface feature data of selected interface features of to a client device 120 corresponding to a user of the medical scan assisted review system for display via a display device of the client device. The medical scan assisted review system 102 can generate scan review data for a medical scan based on user input to the interactive interface displayed by the display device in response to prompts to provide the scan review data, for example, where the prompts correspond to one or more interface features.

The medical scan assisted review system 102 can be operable to receive, via a network, a medical scan for review. Abnormality annotation data can be generated by identifying one or more of abnormalities in the medical scan by utilizing a computer vision model that is trained on a plurality of training medical scans. The abnormality annotation data can include location data and classification data for each of the plurality of abnormalities and/or data that facilitates the visualization of the abnormalities in the scan image data. Report data including text describing each of the plurality of abnormalities is generated based on the abnormality data. The visualization and the report data, which can collectively be displayed annotation data, can be transmitted to a client device. A display device associated with the client device can display the visualization in conjunction with the medical scan via an interactive interface, and the display device can further display the report data via the interactive interface.

In various embodiments, longitudinal data, such as one or more additional scans of longitudinal data 433 of the medical scan or of similar scans, can be displayed in conjunction with the medical scan automatically, or in response to the user electing to view longitudinal data via user input. For example, the medical scan assisted review system can retrieve a previous scan or a future scan for the patient from a patient database or from the medical scan database automatically or in response to the user electing to view past patient data. One or more previous scans can be displayed in one or more corresponding windows adjacent to the current medical scan. For example, the user can select a past scan from the longitudinal data for display. Alternatively or in addition, the user can elect longitudinal parameters such as amount of time elapsed, scan type, electing to select the most recent and/or least recent scan, electing to select a future scan, electing to select a scan at a date closest to the scan, or other criteria, and the medical scan assisted review system can automatically select a previous scan that compares most favorably to the longitudinal parameters. The selected additional scan can be displayed in an adjacent window alongside the current medical scan. In some embodiments, multiple additional scans will be selected and can be displayed in multiple adjacent windows.

In various embodiments, a first window displaying an image slice 412 of the medical scan and an adjacent second window displaying an image slice of a selected additional scan will display image slices 412 determined to correspond with the currently displayed slice 412 of the medical scan. As described with respect to selecting a slice of a selected similar medical scan for display, this can be achieved based on selecting the image slice with a matching slice number, based on automatically determining the image slice that most closely matches the anatomical region corresponding to the currently displayed slice of the current scan, and/or based on determining the slice in the previous scan with the most similar view of the abnormality as the currently displayed slice. The user can use a single scroll bar or other single user input indication to jump to a different image slice, and the multiple windows can simultaneously display the same numbered image slice, or can scroll or jump by the same number of slices if different slice numbers are initially displayed. In some embodiments, three or more adjacent windows corresponding to the medical scan and two or more additional scans are displayed, and can all be controlled with the single scroll bar in a similar fashion.

The medical scan assisted review system 102 can automatically detect previous states of the identified abnormalities based on the abnormality data, such as the abnormality location data. The detected previous states of the identified abnormality can be circled, highlighted, or otherwise indicated in their corresponding window. The medical scan assisted review system 102 can retrieve classification data for the previous state of the abnormality by retrieving abnormality annotation data 442 of the similar abnormality mapped to the previous scan from the medical scan database 342. This data may not be assigned to the previous scan, and the medical scan assisted review system can automatically determine classification or other diagnosis data for the previous medical scan by utilizing the medical scan image analysis system as discussed. Alternatively or in addition, some or all of the abnormality classification data 445 or other diagnosis data 440 for the previous scan can be assigned values determined based on the abnormality classification data or other diagnosis data determined for the current scan. Such abnormality classification data 445 or other diagnosis data 440 determined for the previous scan can be mapped to the previous scan, and or mapped to the longitudinal data 433, in the database and/or transmitted to a responsible entity via the network.

The medical assisted review system can automatically generate state change data such as a change in size, volume, malignancy, or other changes to various classifiers of the abnormality. This can be achieved by automatically comparing image data of one or more previous scans and the current scan and/or by comparing abnormality data of the previous scan to abnormality data of the current scan. In some embodiments, such metrics can be calculated by utilizing the medical scan similarity analysis function, for example, where the output of the medical scan similarity analysis function such as the similarity score indicates distance, error, or other measured discrepancy in one or more abnormality classifier categories 444 and/or abnormality pattern categories 446. This calculated distance, error, or other measured discrepancy in each category can be used to quantify state change data, indicate a new classifier in one or more categories, to determine if a certain category has become more or less severe, or otherwise determine how the abnormality has changed over time. In various embodiments, this data can be displayed in one window, for example, where an increase in abnormality size is indicated by overlaying or highlighting an outline of the current abnormality over the corresponding image slice of the previous abnormality, or vice versa. In various embodiments where several past scans are available, such state change data can be determined over time, and statistical data showing growth rate changes over time or malignancy changes over time can be generated, for example, indicating if a growth rate is lessening or worsening over time. Image slices corresponding to multiple past scans can be displayed in sequence, for example, where a first scroll bar allows a user to scroll between image slice numbers, and a second scroll bar allows a user to scroll between the same image slice over time. In various embodiments the abnormality data, heat map data, or other interface features will be displayed in conjunction with the image slices of the past image data.

The medical scan report labeling system 104 can be used to automatically assign medical codes to medical scans based on user identified keywords, phrases, or other relevant medical condition terms of natural language text data in a medical scan report of the medical scan, identified by users of the medical scan report labeling system 104. The medical scan report labeling system 104 can be operable to transmit a medical report that includes natural language text to a first client device for display. Identified medical condition term data can be received from the first client device in response. An alias mapping pair in a medical label alias database can be identified by determining that a medical condition term of the alias mapping pair compares favorably to the identified medical condition term data. A medical code that corresponds to the alias mapping pair and a medical scan that corresponds to the medical report can be transmitted to a second client device of an expert user for display, and accuracy data can be received from the second client device in response. The medical code is mapped to the first medical scan in a medical scan database when the accuracy data indicates that the medical code compares favorably to the medical scan.

The medical scan annotator system 106 can be used to gather annotations of medical scans based on review of the medical scan image data by users of the system such as radiologists or other medical professionals. Medical scans that require annotation, for example, that have been triaged from a hospital or other triaging entity, can be sent to multiple users selected by the medical scan annotator system 106, and the annotations received from the multiple medical professionals can be processed automatically by a processing system of the medical scan annotator system, allowing the medical scan annotator system to automatically determine a consensus annotation of each medical scan. Furthermore, the users can be automatically scored by the medical scan annotator system based on how closely their annotation matches to the consensus annotation or some other truth annotation, for example, corresponding to annotations of the medical scan assigned a truth flag. Users can be assigned automatically to annotate subsequent incoming medical scans based on their overall scores and/or based on categorized scores that correspond to an identified category of the incoming medical scan.

The medical scan annotator system 106 can be operable to select a medical scan for transmission via a network to a first client device and a second client device for display via an interactive interface, and annotation data can be received from the first client device and the second client device in response. Annotation similarity data can be generated by comparing the first annotation data to the second annotation data, and consensus annotation data can be generated based on the first annotation data and the second annotation data in response to the annotation similarity data indicating that the difference between the first annotation data and the second annotation data compares favorably to an annotation discrepancy threshold. The consensus annotation data can be mapped to the medical scan in a medical scan database.

A medical scan diagnosing system 108 can be used by hospitals, medical professionals, or other medical entities to automatically produce inference data for given medical scans by utilizing computer vision techniques and/or natural language processing techniques. This automatically generated inference data can be used to generate and/or update diagnosis data or other corresponding data of corresponding medical scan entries in a medical scan database. The medical scan diagnosing system can utilize a medical scan database, user database, and/or a medical scan analysis function database by communicating with the database storage system 140 via the network 150, and/or can utilize another medical scan database, user database, and/or function database stored in local memory.

The medical scan diagnosing system 108 can be operable to receive a medical scan. Diagnosis data of the medical scan can be generated by performing a medical scan inference function on the medical scan. The first medical scan can be transmitted to a first client device associated with a user of the medical scan diagnosing system in response to the diagnosis data indicating that the medical scan corresponds to a non-normal diagnosis. The medical scan can be displayed to the user via an interactive interface displayed by a display device corresponding to the first client device. Review data can be received from the first client device, where the review data is generated by the first client device in response to a prompt via the interactive interface. Updated diagnosis data can be generated based on the review data. The updated diagnosis data can be transmitted to a second client device associated with a requesting entity.

A medical scan interface feature evaluating system 110 can be used evaluate proposed interface features or currently used interface features of an interactive interface to present medical scans for review by medical professionals or other users of one or more subsystems 101. The medical scan interface feature evaluator system 110 can be operable to generate an ordered image-to-prompt mapping by selecting a set of user interface features to be displayed with each of an ordered set of medical scans. The set of medical scans and the ordered image-to-prompt mapping can be transmitted to a set of client devices. A set of responses can be generated by each client device in response to sequentially displaying each of the set of medical scans in conjunction with a mapped user interface feature indicated in the ordered image-to-prompt mapping via a user interface. Response score data can be generated by comparing each response to truth annotation data of the corresponding medical scan. Interface feature score data corresponding to each user interface feature can be generated based on aggregating the response score data, and is used to generate a ranking of the set of user interface features.

A medical scan image analysis system 112 can be used to generate and/or perform one or more medical scan image analysis functions by utilizing a computer vision-based learning algorithm 1350 on a training set of medical scans with known annotation data, diagnosis data, labeling and/or medical code data, report data, patient history data, patient risk factor data, and/or other metadata associated with medical scans. These medical scan image analysis functions can be used to generate inference data for new medical scans that are triaged or otherwise require inferred annotation data, diagnosis data, labeling and/or medical code data, and/or report data. For example, some medical scan image analysis functions can correspond to medical scan inference functions of the medical scan diagnosing system or other medical scan analysis functions of a medical scan analysis function database. The medical scan image analysis functions can be used to determine whether or not a medical scan is normal, to detect the location of an abnormality in one or more slices of a medical scan, and/or to characterize a detected abnormality. The medical scan image analysis system can be used to generate and/or perform computer vision based medical scan image analysis functions utilized by other subsystems of the medical scan processing system as described herein, aiding medical professionals to diagnose patients and/or to generate further data and models to characterize medical scans. The medical scan image analysis system can include a processing system that includes a processor and a memory that stores executable instructions that, when executed by the processing system, facilitate performance of operations.

The medical scan image analysis system 112 can be operable to receive a plurality of medical scans that represent a three-dimensional anatomical region and include a plurality of cross-sectional image slices. A plurality of three-dimensional subregions corresponding to each of the plurality of medical scans can be generated by selecting a proper subset of the plurality of cross-sectional image slices from each medical scan, and by further selecting a two-dimensional subregion from each proper subset of cross-sectional image slices. A learning algorithm can be performed on the plurality of three-dimensional subregions to generate a neural network. Inference data corresponding to a new medical scan received via the network can be generated by performing an inference algorithm on the new medical scan by utilizing the neural network. An inferred abnormality can be identified in the new medical scan based on the inference data.

The medical scan natural language analysis system 114 can determine a training set of medical scans with medical codes determined to be truth data. Corresponding medical reports and/or other natural language text data associated with a medical scan can be utilized to train a medical scan natural language analysis function by generating a medical report natural language model. The medical scan natural language analysis function can be utilized to generate inference data for incoming medical reports for other medical scans to automatically determine corresponding medical codes, which can be mapped to corresponding medical scans. Medical codes assigned to medical scans by utilizing the medical report natural language model can be utilized by other subsystems, for example, to train other medical scan analysis functions, to be used as truth data to verify annotations provided via other subsystems, to aid in diagnosis, or otherwise be used by other subsystems as described herein.

A medical scan comparison system 116 can be utilized by one or more subsystems to identify and/or display similar medical scans, for example, to perform or determine function parameters for a medical scan similarity analysis function, to generate or retrieve similar scan data, or otherwise compare medical scan data. The medical scan comparison system 116 can also utilize some or all features of other subsystems as described herein. The medical scan comparison system 116 can be operable to receive a medical scan via a network and can generate similar scan data. The similar scan data can include a subset of medical scans from a medical scan database and can be generated by performing an abnormality similarity function, such as medical scan similarity analysis function, to determine that a set of abnormalities included in the subset of medical scans compare favorably to an abnormality identified in the medical scan. At least one cross-sectional image can be selected from each medical scan of the subset of medical scans for display on a display device associated with a user of the medical scan comparison system in conjunction with the medical scan.

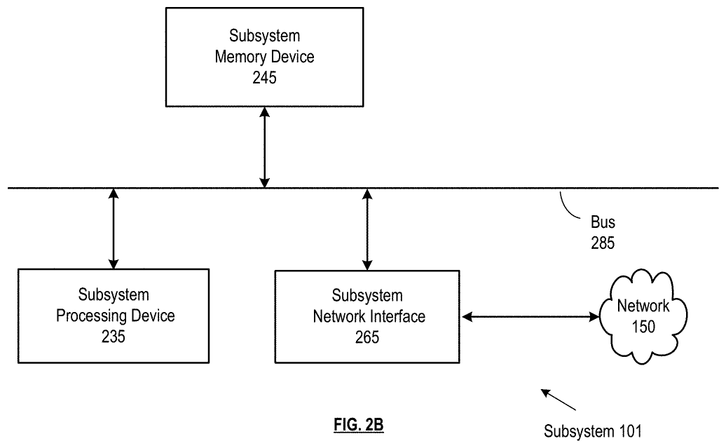

FIG. 2A presents an embodiment of client device 120. Each client device 120 can include one or more client processing devices 230, one or more client memory devices 240, one or more client input devices 250, one or more client network interfaces 260 operable to more support one or more communication links via the network 150 indirectly and/or directly, and/or one or more client display devices 270, connected via bus 280. Client applications 202, 204, 206, 208, 210, 212, 214, and/or 216 correspond to subsystems 102, 104, 106, 108, 110, 112, 114, and/or 116 of the medical scan processing system respectfully. Each client device 120 can receive the application data from the corresponding subsystem via network 150 by utilizing network interface 260, for storage in the one or more memory devices 240. In various embodiments, some or all client devices 120 can include a computing device associated with a radiologist, medical entity, or other user of one or more subsystems as described herein.

The one or more processing devices 230 can display interactive interface 275 on the one or more client display devices 270 in accordance with one or more of the client applications 202, 204, 206, 208, 210, 212, 214, and/or 216, for example, where a different interactive interface 275 is displayed for some or all of the client applications in accordance with the website presented by the corresponding subsystem 102, 104, 106, 108, 110, 112, 114 and/or 116. The user can provide input in response to menu data or other prompts presented by the interactive interface via the one or more client input devices 250, which can include a microphone, mouse, keyboard, touchscreen of display device 270 itself or other touchscreen, and/or other device allowing the user to interact with the interactive interface. The one or more processing devices 230 can process the input data and/or send raw or processed input data to the corresponding subsystem, and/or can receive and/or generate new data in response for presentation via the interactive interface 275 accordingly, by utilizing network interface 260 to communicate bidirectionally with one or more subsystems and/or databases of the medical scan processing system via network 150.

FIG. 2B presents an embodiment of a subsystem 101, which can be utilized in conjunction with subsystem 102, 104, 106, 108, 110, 112, 114 and/or 116. Each subsystem 101 can include one or more subsystem processing devices 235, one or more subsystem memory devices 245, and/or one or more subsystem network interfaces 265, connected via bus 285. The subsystem memory devices 245 can store executable instructions that, when executed by the one or more subsystem processing devices 235, facilitate performance of operations by the subsystem 101, as described for each subsystem herein.

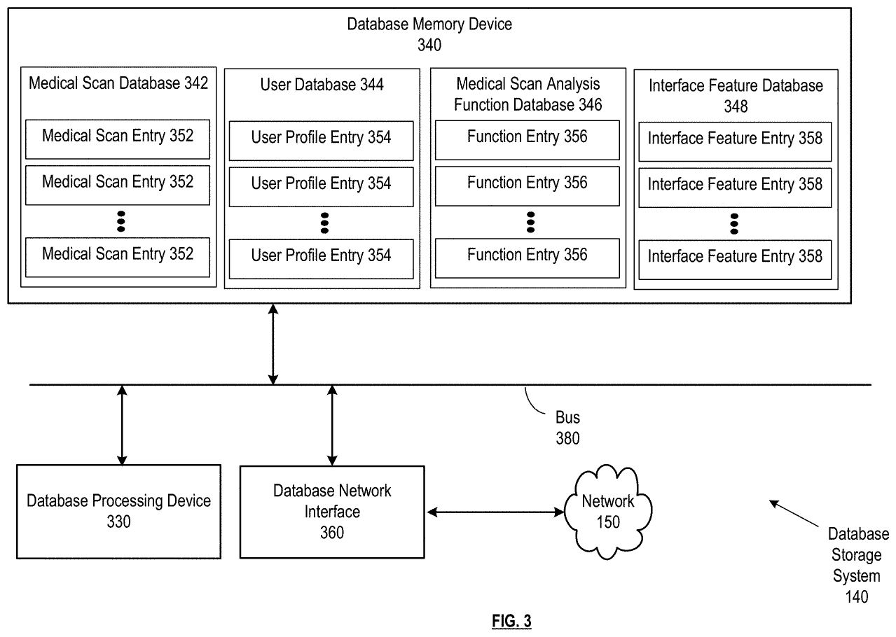

FIG. 3 presents an embodiment of the database storage system 140. Database storage system 140 can include at least one database processing device 330, at least one database memory device 340, and at least one database network interface 360, operable to more support one or more communication links via the network 150 indirectly and/or directly, all connected via bus 380. The database storage system 140 can store one or more databases the at least one memory 340, which can include a medical scan database 342 that includes a plurality medical scan entries 352, a user database 344 that includes a plurality of user profile entries 354, a medical scan analysis function database 346 that includes a plurality of medical scan analysis function entries 356, an interface feature database 348 can include a plurality of interface feature entries 358, and/or other databases that store data generated and/or utilized by the subsystems 101. Some or all of the databases 342, 344, 346 and/or 348 can consist of multiple databases, can be stored relationally or non-relationally, and can include different types of entries and different mappings than those described herein. A database entry can include an entry in a relational table or entry in a non-relational structure. Some or all of the data attributes of an entry 352, 354, 356, and/or 358 can refer to data included in the entry itself or that is otherwise mapped to an identifier included in the entry and can be retrieved from, added to, modified, or deleted from the database storage system 140 based on a given identifier of the entry. Some or all of the databases 342, 344, 346, and/or 348 can instead be stored locally by a corresponding subsystem, for example, if they are utilized by only one subsystem.

The processing device 330 can facilitate read/write requests received from subsystems and/or client devices via the network 150 based on read/write permissions for each database stored in the at least one memory device 340. Different subsystems can be assigned different read/write permissions for each database based on the functions of the subsystem, and different client devices 120 can be assigned different read/write permissions for each database. One or more client devices 120 can correspond to one or more administrators of one or more of the databases stored by the database storage system, and database administrator devices can manage one or more assigned databases, supervise assess and/or efficiency, edit permissions, or otherwise oversee database processes based on input to the client device via interactive interface 275.

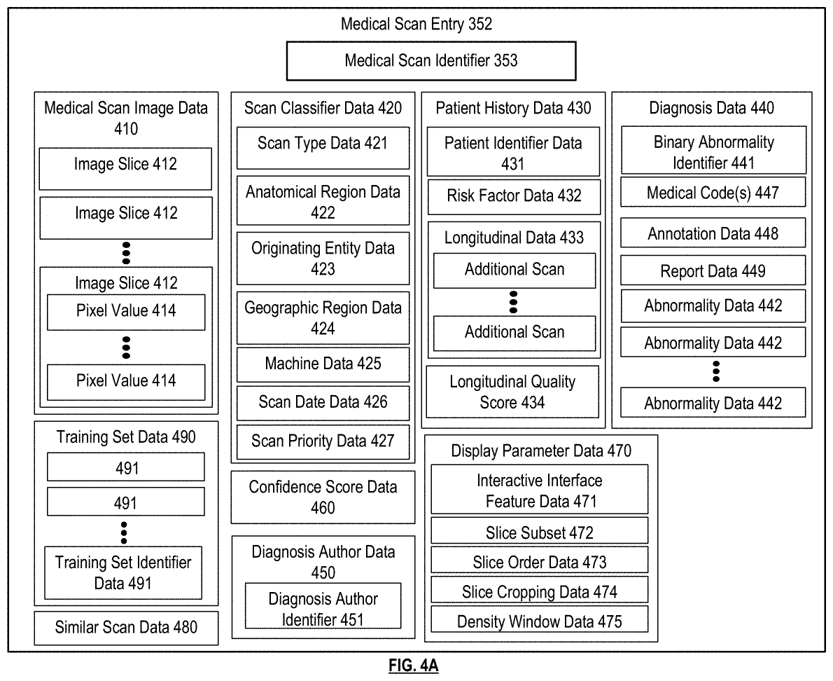

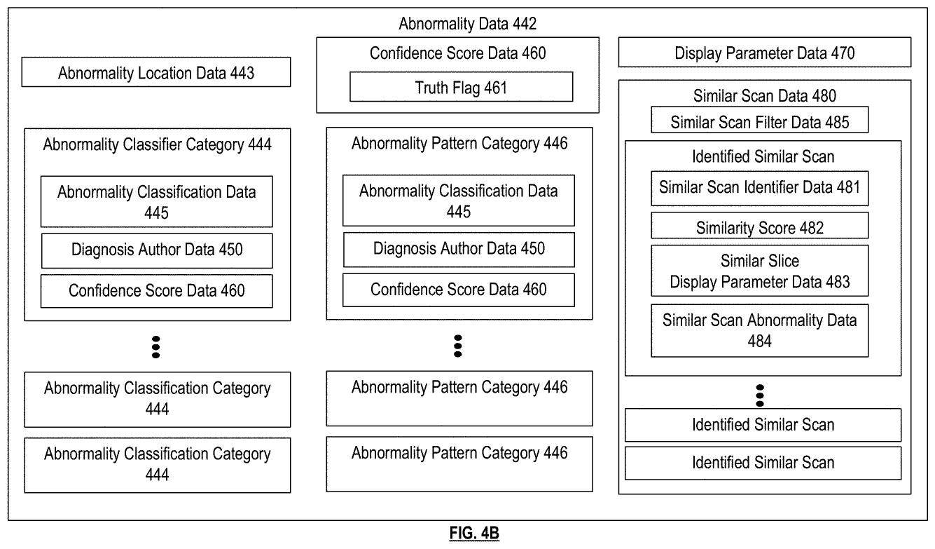

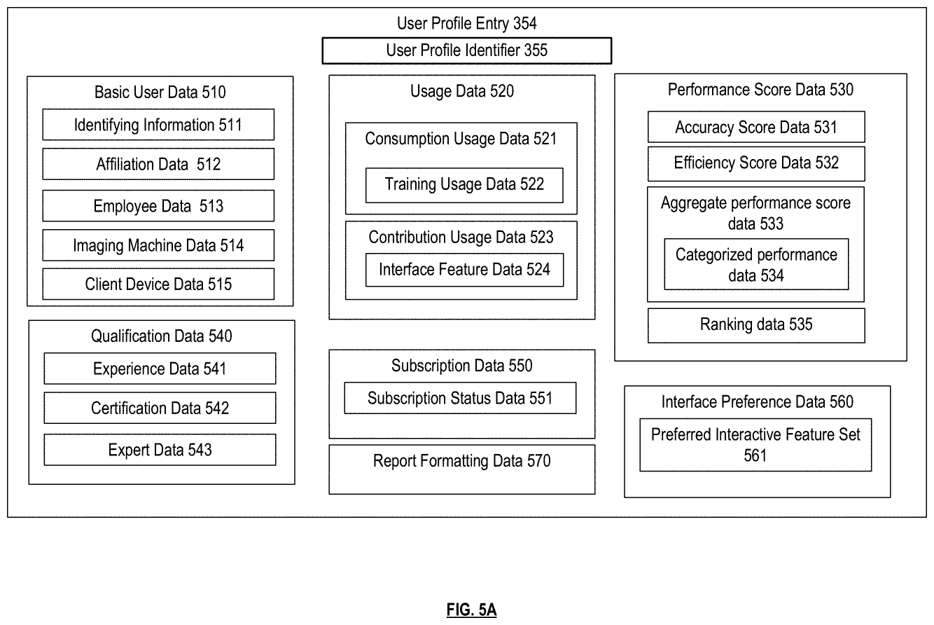

FIG. 4A presents an embodiment of a medical scan entry 352, stored in medical scan database 342, included in metadata of a medical scan, and/or otherwise associated with a medical scan. A medical scan can include imaging data corresponding to a CT scan, x-ray, MRI, PET scan, Ultrasound, EEG, mammogram, or other type of radiological scan or medical scan taken of an anatomical region of a human body, animal, organism, or object and further can include metadata corresponding to the imaging data. Some or all of the medical scan entries can be formatted in accordance with a Digital Imaging and Communications in Medicine (DICOM) format or other standardized image format, and some or more of the fields of the medical scan entry 352 can be included in a DICOM header or other standardized header of the medical scan. Medical scans can be awaiting review or can have already been reviewed by one or more users or automatic processes and can include tentative diagnosis data automatically generated by a subsystem, generated based on user input, and/or generated from another source. Some medical scans can include final, known diagnosis data generated by a subsystem and/or generated based on user input, and/or generated from another source, and can included in training sets used to train processes used by one or more subsystems such as the medical scan image analysis system 112 and/or the medical scan natural language analysis system 114.

Some medical scans can include one or more abnormalities, which can be identified by a user or can be identified automatically. Abnormalities can include nodules, for example malignant nodules identified in a chest CT scan. Abnormalities can also include and/or be characterized by one or more abnormality pattern categories such as such as cardiomegaly, consolidation, effusion, emphysema, and/or fracture, for example identified in a chest x-ray. Abnormalities can also include any other unknown, malignant or benign feature of a medical scan identified as not normal. Some scans can contain zero abnormalities, and can be identified as normal scans. Some scans identified as normal scans can include identified abnormalities that are classified as benign, and include zero abnormalities classified as either unknown or malignant. Scans identified as normal scans may include abnormalities that were not detected by one or more subsystems and/or by an originating entity. Thus, some scans may be improperly identified as normal. Similarly, scans identified to include at least one abnormality may include at least one abnormality that was improperly detected as an abnormality by one or more subsystems and/or by an originating entity. Thus, some scans may be improperly identified as containing abnormalities.

Each medical scan entry 352 can be identified by its own medical scan identifier 353, and can include or otherwise map to medical scan image data 410, and metadata such as scan classifier data 420, patient history data 430, diagnosis data 440, annotation author data 450, confidence score data 460, display parameter data 470, similar scan data 480, training set data 490, and/or other data relating to the medical scan. Some or all of the data included in a medical scan entry 352 can be used to aid a user in generating or editing diagnosis data 440, for example, in conjunction with the medical scan assisted review system 102, the medical scan report labeling system 104, and/or the medical scan annotator system 106. Some or all of the data included in a medical scan entry 352 can be used to allow one or more subsystems 101, such as automated portions of the medical scan report labeling system 104 and/or the medical scan diagnosing system 108, to automatically generate and/or edit diagnosis data 440 or other data the medical scan. Some or all of the data included in a medical scan entry 352 can be used to train some or all medical scan analysis functions of the medical scan analysis function database 346 such as one or more medical scan image analysis functions, one or more medical scan natural language analysis functions, one or more medical scan similarity analysis functions, one or more medical report generator functions, and/or one or more medical report analysis functions, for example, in conjunction with the medical scan image analysis system 112, the medical scan natural language analysis system 114, and/or the medical scan comparison system 116.

The medical scan entries 352 and the associated data as described herein can also refer to data associated with a medical scan that is not stored by the medical scan database, for example, that is uploaded by a client device for direct transmission to a subsystem, data generated by a subsystem and used as input to another subsystem or transmitted directly to a client device, data stored by a Picture Archive and Communication System (PACS) communicating with the medical scan processing system 100, or other data associated with a medical scan that is received and or generated without being stored in the medical scan database 342. For example, some or all of the structure and data attributes described with respect to a medical scan entry 352 can also correspond to structure and/or data attribute of data objects or other data generated by and/or transmitted between subsystems and/or client devices that correspond to a medical scan. Herein, any of the data attributes described with respect to a medical scan entry 352 can also correspond to data extracted from a data object generated by a subsystem or client device or data otherwise received from a subsystem, client device, or other source via network 150 that corresponds to a medical scan.

The medical scan image data 410 can include one or more images corresponding to a medical scan. The medical scan image data 410 can include one or more image slices 412, for example, corresponding to a single x-ray image, a plurality of cross-sectional, tomographic images of a scan such as a CT scan, or any plurality of images taken from the same or different point at the same or different angles. The medical scan image data 410 can also indicate an ordering of the one or more image slices 412. Herein, a “medical scan” can refer a full scan of any type represented by medical scan image data 410. Herein, an “image slice” can refer to one of a plurality of cross-sectional images of the medical scan image data 410, one of a plurality of images taken from different angles of the medical scan image data 410, and/or the single image of the medical scan image data 410 that includes only one image. Furthermore “plurality of image slices” can refer to all of the images of the associated medical scan, and refers to only a single image if the medical scan image data 410 includes only one image. Each image slice 412 can include a plurality of pixel values 414 mapped to each pixel of the image slice. Each pixel value can correspond to a density value, such as a Hounsfield value or other measure of density. Pixel values can also correspond to a grayscale value, an RGB (Red-Green-Blue) or other color value, or other data stored by each pixel of an image slice 412.

Scan classifier data 420 can indicate classifying data of the medical scan. Scan classifier data can include scan type data 421, for example, indicating the modality of the scan. The scan classifier data can indicate that the scan is a CT scan, x-ray, MRI, PET scan, Ultrasound, EEG, mammogram, or other type of scan. Scan classifier data 420 can also include anatomical region data 422, indicating for example, the scan is a scan of the chest, head, right knee, or other anatomical region. Scan classifier data can also include originating entity data 423, indicating the hospital where the scan was taken and/or a user that uploaded the scan to the system. If the originating entity data corresponds to a user of one or more subsystems 101, the originating entity data can include a corresponding user profile identifier and/or include other data from the user profile entry 354 of the user. Scan classifier data 420 can include geographic region data 424, indicating a city, state, and/or country from which the scan originated, for example, based on the user data retrieved from the user database 344 based on the originating entity. Scan classifier data can also include machine data 425, which can include machine identifier data, machine model data, machine calibration data, and/or contrast agent data, for example based on imaging machine data retrieved from the user database 344 based on the originating entity data 423. The scan classifier data 420 can include scan date data 426 indicating when the scan was taken. The scan classifier data 420 can include scan priority data 427, which can indicate a priority score, ranking, number in a queue, or other priority data with regard to triaging and/or review. A priority score, ranking, or queue number of the scan priority data 427 can be generated by automatically by a subsystem based on the scan priority data 427, based on a severity of patient symptoms or other indicators in the risk factor data 432, based on a priority corresponding to the originating entity, based on previously generated diagnosis data 440 for the scan, and/or can be assigned by the originating entity and/or a user of the system.

The scan classifier data 420 can include other classifying data not pictured in FIG. 4A. For example, a set of scans can include medical scan image data 410 corresponding to different imaging planes. The scan classifier data can further include imaging plane data indicating one or more imaging planes corresponding to the image data. For example, the imaging plane data can indicate the scan corresponds to the axial plane, sagittal plane, or coronal plane. A single medical scan entry 352 can include medical scan image data 410 corresponding multiple planes, and each of these planes can be tagged appropriately in the image data. In other embodiments, medical scan image data 410 corresponding to each plane can be stored as separate medical scan entries 352, for example, with a common identifier indicating these entries belong to the same set of scans.

Alternatively or in addition, the scan classifier data 420 can include sequencing data. For example, a set of scans can include medical scan image data 410 corresponding to different sequences. The scan classifier data can further include sequencing data indicating one or more of a plurality of sequences of the image data corresponds to, for example, indicating whether an MRI scan corresponds to a T2 sequence, a T1 sequence, a T1 sequence with contrast, a diffusion sequence, a FLAIR sequence, or other MRI sequence. A single medical scan entry 352 can include medical scan image data 410 corresponding to multiple sequences, and each of these sequences can be tagged appropriately in the entry. In other embodiments, medical scan image data 410 corresponding to each sequence can be stored as separate medical scan entries 352, for example, with a common identifier indicating these entries belong to the same set of scans.

Alternatively or in addition, the scan classifier data 420 can include an image quality score. This score can be determined automatically by one or more subsystems 101, and/or can be manually assigned the medical scan. The image quality score can be based on a resolution of the image data 410, where higher resolution image data is assigned a more favorable image quality score than lower resolution image data. The image quality score can be based on whether the image data 410 corresponds to digitized image data received directly from the corresponding imaging machine, or corresponds to a hard copy of the image data that was later scanned in. In some embodiments, the image quality score can be based on a detected corruption, and/or detected external factor that determined to negatively affect the quality of the image data during the capturing of the medical scan and/or subsequent to the capturing of the medical scan. In some embodiments, the image quality score can be based on detected noise in the image data, where a medical scan with a higher level of detected noise can receive a less favorable image quality score than a medical scan with a lower level of detected noise. Medical scans with this determined corruption or external factor can receive a less favorable image quality score than medical scans with no detected corruption or external factor.

In some embodiments, the image quality score can be based on include machine data 425. In some embodiments, one or more subsystems can utilize the image quality score to flag medical scans with image quality scores that fall below an image quality threshold. The image quality threshold can be the same or different for different subsystems, medical scan modalities, and/or anatomical regions. For example, the medical scan image analysis system can automatically filter training sets based on selecting only medical scans with image quality scores that compare favorably to the image quality threshold. As another example, one or more subsystems can flag a particular imaging machine and/or hospital or other medical entity that have produced at least a threshold number and/or percentage of medical scan with image quality scores that compare unfavorably to the image quality threshold. As another example, a de-noising algorithm can be automatically utilized to clean the image data when the image quality score compares unfavorably to the image quality threshold. As another example, the medical scan image analysis system can select a particular medical image analysis function from a set of medical image analysis functions to utilize on a medical scan to generate inference data for the medical scan. Each of this set of medical image analysis function can be trained on different levels of image quality, and the selected image analysis function can be selected based on the determined image quality score falling within a range of image quality scores the image analysis function was trained on and/or is otherwise suitable for.

The patient history data 430 can include patient identifier data 431 which can include basic patient information such as name or an identifier that may be anonymized to protect the confidentiality of the patient, age, and/or gender. The patient identifier data 431 can also map to a patient entry in a separate patient database stored by the database storage system, or stored elsewhere. The patient history data can include patient risk factor data 432 which can include previous medical history, family medical history, smoking and/or drug habits, pack years corresponding to tobacco use, environmental exposures, patient symptoms, etc. The patient history data 430 can also include longitudinal data 433, which can identify one or more additional medical scans corresponding to the patient, for example, retrieved based on patient identifier data 431 or otherwise mapped to the patient identifier data 431. Some or all additional medical scans can be included in the medical scan database, and can be identified based on their corresponding identifiers medical scan identifiers 353. Some or all additional medical scans can be received from a different source and can otherwise be identified. Alternatively or in addition, the longitudinal data can simply include some or all relevant scan entry data of a medical scan entry 352 corresponding to the one or more additional medical scans. The additional medical scans can be the same type of scan or different types of scans. Some or all of the additional scans may correspond to past medical scans, and/or some or all of the additional scans may correspond to future medical scans. The longitudinal data 433 can also include data received and/or determined at a date after the scan such as final biopsy data, or some or all of the diagnosis data 440. The patient history data can also include a longitudinal quality score 434, which can be calculated automatically by a subsystem, for example, based on the number of additional medical scans, based on how many of the additional scans in the file were taken before and/or after the scan based on the scan date data 426 of the medical scan and the additional medical scans, based on a date range corresponding to the earliest scan and corresponding to the latest scan, based on the scan types data 421 these scans, and/or based on whether or not a biopsy or other final data is included. As used herein, a “high” longitudinal quality score refers to a scan having more favorable longitudinal data than that with a “low” longitudinal quality score.

Diagnosis data 440 can include data that indicates an automated diagnosis, a tentative diagnosis, and/or data that can otherwise be used to support medical diagnosis, triage, medical evaluation and/or other review by a medical professional or other user. The diagnosis data 440 of a medical scan can include a binary abnormality identifier 441 indicating whether the scan is normal or includes at least one abnormality. In some embodiments, the binary abnormality identifier 441 can be determined by comparing some or all of confidence score data 460 to a threshold, can be determined by comparing a probability value to a threshold, and/or can be determined by comparing another continuous or discrete value indicating a calculated likelihood that the scan contains one or more abnormalities to a threshold. In some embodiments, non-binary values, such as one or more continuous or discrete values indicating a likelihood that the scan contains one or more abnormalities, can be included in diagnosis data 440 in addition to, or instead of, binary abnormality identifier 441. One or abnormalities can be identified by the diagnosis data 440, and each identified abnormality can include its own set of abnormality annotation data 442. Alternatively, some or all of the diagnosis data 440 can indicate and/or describe multiple abnormalities, and thus will not be presented for each abnormality in the abnormality annotation data 442. For example, the report data 449 of the diagnosis data 440 can describe all identified abnormalities, and thus a single report can be included in the diagnosis.

FIG. 4B presents an embodiment of the abnormality annotation data 442. The abnormality annotation data 442 for each abnormality can include abnormality location data 443, which can include an anatomical location and/or a location specific to pixels, image slices, coordinates or other location information identifying regions of the medical scan itself. The abnormality annotation data 442 can include abnormality classification data 445 which can include binary, quantitative, and/or descriptive data of the abnormality as a whole, or can correspond to one or more abnormality classifier categories 444, which can include size, volume, pre-post contrast, doubling time, calcification, components, smoothness, spiculation, lobulation, sphericity, internal structure, texture, or other categories that can classify and/or otherwise characterize an abnormality. Abnormality classifier categories 444 can be assigned a binary value, indicating whether or not such a category is present. For example, this binary value can be determined by comparing some or all of confidence score data 460 to a threshold, can be determined by comparing a probability value to a threshold, and/or can be determined by comparing another continuous or discrete value indicating a calculated likelihood that a corresponding abnormality classifier category 444 is present to a threshold, which can be the same or different threshold for each abnormality classifier category 444. In some embodiments, abnormality classifier categories 444 can be assigned one or more non-binary values, such as one or more continuous or discrete values indicating a likelihood that the corresponding classifier category 444 is present.

The abnormality classifier categories 444 can also include a malignancy category, and the abnormality classification data 445 can include a malignancy rating such as a Lung-RADS score, a Fleischner score, and/or one or more calculated values that indicate malignancy level, malignancy severity, and/or probability of malignancy. Alternatively or in addition, the malignancy category can be assigned a value of “yes”, “no”, or “maybe”. The abnormality classifier categories 444 can also include abnormality pattern categories 446 such as cardiomegaly, consolidation, effusion, emphysema, and/or fracture, and the abnormality classification data 445 for each abnormality pattern category 446 can indicate whether or not each of the abnormality patterns is present.

The abnormality classifier categories can correspond to Response Evaluation Criteria in Solid Tumors (RECIST) eligibility and/or RECIST evaluation categories. For example, an abnormality classifier category 444 corresponding to RECIST eligibility can have corresponding abnormality classification data 445 indicating a binary value “yes” or “no”, and/or can indicate if the abnormality is a “target lesion” and/or a “non-target lesion.” As another example, an abnormality classifier category 444 corresponding to a RECIST evaluation category can be determined based on longitudinal data 433 and can have corresponding abnormality classification data 445 that includes one of the set of possible values “Complete Response”, “Partial Response”, “Stable Disease”, or “Progressive Disease.”

The diagnosis data 440 as a whole, and/or the abnormality annotation data 442 for each abnormality, can include custom codes or datatypes identifying the binary abnormality identifier 441, abnormality location data 443 and/or some or all of the abnormality classification data 445 of one or more abnormality classifier categories 444. Alternatively or in addition, some or all of the abnormality annotation data 442 for each abnormality and/or other diagnosis data 440 can be presented in a DICOM format or other standardized image annotation format, and/or can be extracted into custom datatypes based on abnormality annotation data originally presented in DICOM format. Alternatively or in addition, the diagnosis data 440 and/or the abnormality annotation data 442 for each abnormality can be presented as one or more medical codes 447 such as SNOMED codes, Current Procedure Technology (CPT) codes, ICD-9 codes, ICD-10 codes, or other standardized medical codes used to label or otherwise describe medical scans.

Alternatively or in addition, the diagnosis data 440 can include natural language text data 448 annotating or otherwise describing the medical scan as a whole, and/or the abnormality annotation data 442 can include natural language text data 448 annotating or otherwise describing each corresponding abnormality. In some embodiments, some or all of the diagnosis data 440 is presented only as natural language text data 448. In some embodiments, some or all of the diagnosis data 440 is automatically generated by one or more subsystems based on the natural language text data 448, for example, without utilizing the medical scan image data 410, for example, by utilizing one or more medical scan natural language analysis functions trained by the medical scan natural language analysis system 114. Alternatively or in addition, some embodiments, some or all of the natural language text data 448 is generated automatically based on other diagnosis data 440 such as abnormality annotation data 442, for example, by utilizing a medical scan natural language generating function trained by the medical scan natural language analysis system 114.

The diagnosis data can include report data 449 that includes at least one medical report, which can be formatted to include some or all of the medical codes 447, some or all of the natural language text data 448, other diagnosis data 440, full or cropped images slices formatted based on the display parameter data 470 and/or links thereto, full or cropped images slices or other data based on similar scans of the similar scan data 480 and/or links thereto, full or cropped images or other data based on patient history data 430 such as longitudinal data 433 and/or links thereto, and/or other data or links to data describing the medical scan and associated abnormalities. The diagnosis data 440 can also include finalized diagnosis data corresponding to future scans and/or future diagnosis for the patient, for example, biopsy data or other longitudinal data 433 determined subsequently after the scan. The medical report of report data 449 can be formatted based on specified formatting parameters such as font, text size, header data, bulleting or numbering type, margins, file type, preferences for including one or more full or cropped image slices 412, preferences for including similar medical scans, preferences for including additional medical scans, or other formatting to list natural language text data and/or image data, for example, based on preferences of a user indicated in the originating entity data 423 or other responsible user in the corresponding report formatting data.

Annotation author data 450 can be mapped to the diagnosis data for each abnormality, and/or mapped to the scan as a whole. This can include one or more annotation author identifiers 451, which can include one or more user profile identifiers of a user of the system, such as an individual medical professional, medical facility and/or medical entity that uses the system. Annotation author data 450 can be used to determine the usage data of a user profile entry 354. Annotation author data 450 can also include one or more medical scan analysis function identifiers 357 or other function identifier indicating one or more functions or other processes of a subsystem responsible for automatically generating and/or assisting a user in generating some or all of the diagnosis data, for example an identifier of a particular type and/or version of a medical scan image analysis functions that was used by the medical scan diagnosing system 108 used to generate part or all of the diagnosis data 440 and/or an interface feature identifier, indicating an one or more interface features presented to a user to facilitate entry of and/or reviewing of the diagnosis data 440. The annotation author data can also simply indicate, for one or more portions of the diagnosis data 440, if this portion was generated by a human or automatically generated by a subsystem of the medical scan processing system.

In some embodiments, if a medical scan was reviewed by multiple entities, multiple, separate diagnosis data entries 440 can be included in the medical scan entry 352, mapped to each diagnosis author in the annotation author data 450. This allows different versions of diagnosis data 440 received from multiple entities. For example, annotation author data of a particular medical scan could indicate that the annotation data was written by a doctor at medical entity A, and the medical code data was generated by user Y by utilizing the medical scan report labeling system 104, which was confirmed by expert user X. The annotation author data of another medical scan could indicate that the medical code was generated automatically by utilizing version 7 of the medical scan image analysis function relating to chest x-rays, and confirmed by expert user X. The annotation author data of another medical scan could indicate that the location and a first malignancy rating were generated automatically by utilizing version 7 of the medical scan image analysis function relating to chest x-rays, and that a second malignancy rating was entered by user Z. In some embodiments, one of the multiple diagnosis entries can include consensus annotation data, for example, generated automatically by a subsystem such as the medical scan annotating system 106 based on the multiple diagnosis data 440, based on confidence score data 460 of each of the multiple diagnosis data 440, and/or based on performance score data of a corresponding user, a medical scan analysis function, or an interface feature, identified in the annotation author data for each corresponding one of the multiple diagnosis data 440.

Confidence score data 460 can be mapped to some or all of the diagnosis data 440 for each abnormality, and/or for the scan as a whole. This can include an overall confidence score for the diagnosis, a confidence score for the binary indicator of whether or not the scan was normal, a confidence score for the location a detected abnormality, and/or confidence scores for some or all of the abnormality classifier data. This may be generated automatically by a subsystem, for example, based on the annotation author data and corresponding performance score of one or more identified users and/or subsystem attributes such as interactive interface types or medical scan image analysis functions indicated by the annotation author data. In the case where multiple diagnosis data entries 440 are included from different sources, confidence score data 460 can be computed for each entry and/or an overall confidence score, for example, corresponding to consensus diagnosis data, can be based on calculated distance or other error and/or discrepancies between the entries, and/or can be weighted on the confidence score data 460 of each entry. In various embodiments, the confidence score data 460 can include a truth flag 461 indicating the diagnosis data is considered as “known” or “truth”, for example, flagged based on user input, flagged automatically based on the author data, and/or flagged automatically based on the calculated confidence score of the confidence score data exceeding a truth threshold. As used herein, a “high” confidence score refers to a greater degree or more favorable level of confidence than a “low” confidence score.

Display parameter data 470 can indicate parameters indicating an optimal or preferred display of the medical scan by an interactive interface 275 and/or formatted report for each abnormality and/or for the scan as a whole. Some or all of the display parameter data can have separate entries for each abnormality, for example, generated automatically by a subsystem 101 based on the abnormality annotation data 442. Display parameter data 470 can include interactive interface feature data 471, which can indicate one or more selected interface features associated with the display of abnormalities and/or display of the medical scan as a whole, and/or selected interface features associated with user interaction with a medical scan, for example, based on categorized interface feature performance score data and a category associated with the abnormality and/or with the medical scan itself. The display parameter data can include a slice subset 472, which can indicate a selected subset of the plurality of image slices that includes a single image slice 412 or multiple image slices 412 of the medical scan image data 410 for display by a user interface. The display parameter data 470 can include slice order data 473 that indicates a selected custom ordering and/or ranking for the slice subset 472, or for all of the slices 412 of the medical scan. The display parameter data 470 can include slice cropping data 474 corresponding to some or all of the slice subset 472, or all of the image slices 412 of the medical scan, and can indicating a selected custom cropped region of each image slice 412 for display, or the same selected custom cropped region for the slice subset 472 or for all slices 412. The display parameter data can include density window data 475, which can indicate a selected custom density window for display of the medical scan as a whole, a selected custom density window for the slice subset 472, and/or selected custom density windows for each of the image slices 412 of the slice subset 472, and/or for each image slice 412 of the medical scan. The density window data 475 can indicate a selected upper density value cut off and a selected lower density value cut off, and/or can include a selected deterministic function to map each density value of a pixel to a grayscale value based on the preferred density window. The interactive interface feature data 471, slice subset 472, slice order data 473, slice cropping data 474, and/or the density window data 475 can be selected via user input and/or generated automatically by one or more subsystems 101, for example, based on the abnormality annotation data 442 and/or based on performance score data of different interactive interface versions.

Similar scan data 480 can be mapped to each abnormality, or the scan as a whole, and can include similar scan identifier data 481 corresponding to one or more identified similar medical scans, for example, automatically identified by a subsystem 101, for example, by applying a similar scan identification step of the medical scan image analysis system 112 and/or applying medical scan similarity analysis function to some or all of the data stored in the medical scan entry of the medical scan, and/or to some or all corresponding data of other medical scans in the medical scan database. The similar scan data 480 can also correspond to medical scans received from another source. The stored similarity data can be used to present similar cases to users of the system and/or can be used to train medical scan image analysis functions or medical scan similarity analysis functions.

Each identified similar medical scan can have its own medical scan entry 352 in the medical scan database 342 with its own data, and the similar scan identifier data 481 can include the medical scan identifier 353 each similar medical scan. Each identified similar medical scan can be a scan of the same scan type or different scan type than medical scan.

The similar scan data 480 can include a similarity score 482 for each identified similar scan, for example, generated based on some or all of the data of the medical scan entry 352 for medical scan and based on some or all of the corresponding data of the medical scan entry 352 for the identified similar medical scan. For example, the similarity score 482 can be generated based on applying a medical scan similarity analysis function to the medical image scan data of medical scans and 402, to some or all of the abnormality annotation data of medical scans and 402, and/or to some or all of the patient history data 430 of medical scans and 402 such as risk factor data 432. As used herein, a “high” similarity score refers a higher level of similarity that a “low” similarity score.

The similar scan data 480 can include its own similar scan display parameter data 483, which can be determined based on some or all of the display parameter data 470 of the identified similar medical scan. Some or all of the similar scan display parameter data 483 can be generated automatically by a subsystem, for example, based on the display parameter data 470 of the identified similar medical scan, based on the abnormality annotation data 442 of the medical scan itself and/or based on display parameter data 470 of the medical scan itself. Thus, the similar scan display parameter data 483 can be the same or different than the display parameter data 470 mapped to the identified similar medical scan and/or can be the same or different than the display parameter data 470 of the medical scan itself. This can be utilized when displaying similar scans to a user via interactive interface 275 and/or can be utilized when generating report data 449 that includes similar scans, for example, in conjunction with the medical scan assisted review system 102.

The similar scan data 480 can include similar scan abnormality data 484, which can indicate one of a plurality of abnormalities of the identified similar medical scan and its corresponding abnormality annotation data 442. For example, the similarity scan abnormality data 484 can include an abnormality pair that indicates one of a plurality of abnormalities of the medical scan, and indicates one of a plurality of abnormalities of the identified similar medical scan, for example, that was identified as the similar abnormality.

The similar scan data 480 can include similar scan filter data 485. The similar scan filter data can be generated automatically by a subsystem, and can include a selected ordered or un-ordered subset of all identified similar scans of the similar scan data 480, and/or a ranking of all identified similar scans. For example, the subset can be selected and/or some or all identified similar scans can be ranked based on each similarity score 482, and/or based on other factors such as based on a longitudinal quality score 434 of each identified similar medical scan.

The training set data 490 can indicate one or more training sets that the medical scan belongs to. For example, the training set data can indicate one or more training set identifiers 491 indicating one or more medical scan analysis functions that utilized the medical scan in their training set, and/or indicating a particular version identifier 641 of the one or more medical scan analysis functions that utilized the medical scan in their training set. The training set data 490 can also indicate which portions of the medical scan entry were utilized by the training set, for example, based on model parameter data 623 of the corresponding medical scan analysis functions. For example, the training set data 490 can indicate that the medical scan image data 410 was included in the training set utilized to train version X of the chest x-ray medical scan image analysis function, or that the natural language text data 448 of this medical scan was used to train version Y of the natural language analysis function.

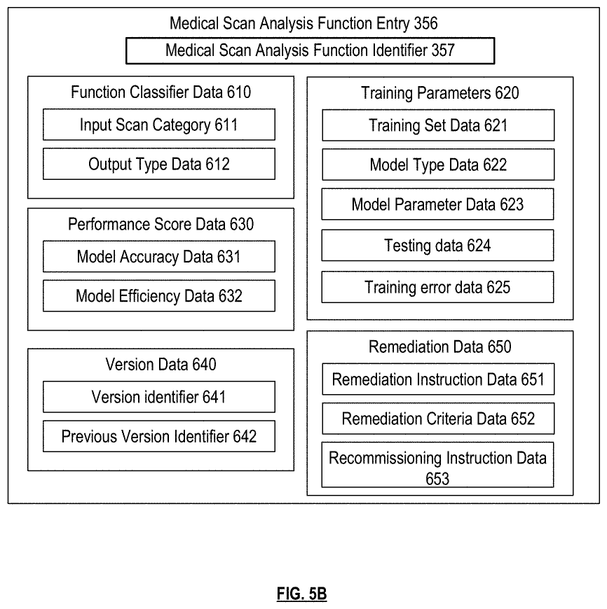

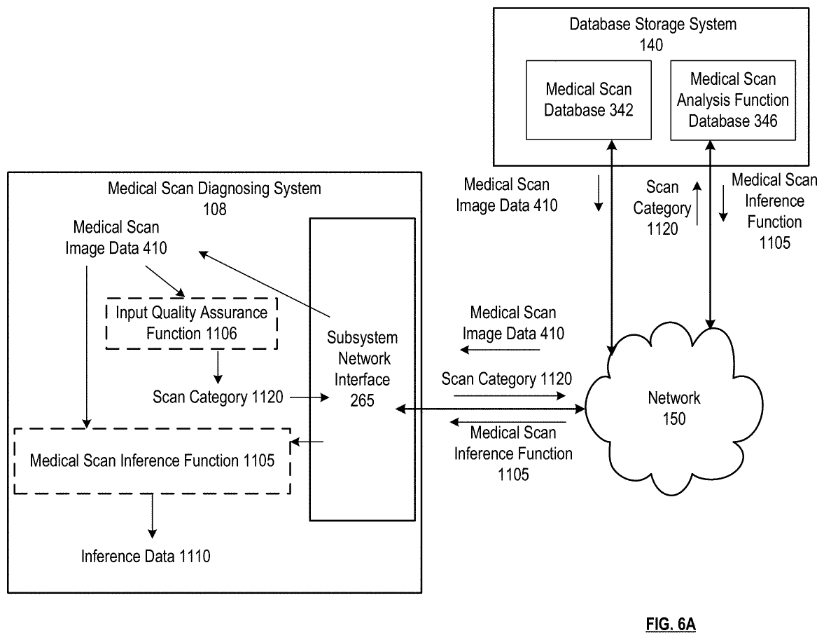

FIG. 5A presents an embodiment of a user profile entry 354, stored in user database 344 or otherwise associated with a user. A user can correspond to a user of one or more of the subsystems such as a radiologist, doctor, medical professional, medical report labeler, administrator of one or more subsystems or databases, or other user that uses one or more subsystems 101. A user can also correspond to a medical entity such as a hospital, medical clinic, establishment that utilizes medical scans, establishment that employs one or more of the medical professionals described, an establishment associated with administering one or more subsystems, or other entity. A user can also correspond to a particular client device 120 or account that can be accessed one or more medical professionals or other employees at the same or different medical entities. Each user profile entry can have a corresponding user profile identifier 355.

A user profile entry 354 can include basic user data 510, which can include identifying information 511 corresponding to the user such as a name, contact information, account/login/password information, geographic location information such as geographic region data 424, and/or other basic information. Basic user data 510 can include affiliation data 512, which can list one or more medical entities or other establishments the user is affiliated with, for example, if the user corresponds to a single person such as a medical professional, or if the user corresponds to a hospital in a network of hospitals. The affiliation data 512 can include one or more corresponding user profile identifiers 355 and/or basic user data 510 if the corresponding affiliated medical entity or other establishment has its own entry in the user database. The user identifier data can include employee data 513 listing one or more employees, such as medical professionals with their own user profile entries 354, for example, if the user corresponds to a medical entity or supervising medical professional of other medical professional employees, and can list a user profile identifier 355 and/or basic user data 510 for each employee. The basic user data 510 can also include imaging machine data 514, which can include a list of machines affiliated with the user which can include machine identifiers, model information, calibration information, scan type information, or other data corresponding to each machine, for example, corresponding to the machine data 425. The user profile entry can include client device data 515, which can include identifiers for one or more client devices associated with the user, for example, allowing subsystems 101 to send data to a client device 120 corresponding to a selected user based on the client device data and/or to determine a user that data was received by determining the client device from which the data was received.

The user profile entry can include usage data 520 which can include identifying information for a plurality of usages by the user in conjunction with using one or more subsystems 101. This can include consumption usage data 521, which can include a listing of, or aggregate data associated with, usages of one or more subsystems by the user, for example, where the user is utilizing the subsystem as a service. For example, the consumption usage data 521 can correspond to each instance where diagnosis data was sent to the user for medical scans provided to the user in conjunction with the medical scan diagnosing system 108 and/or the medical scan assisted review system 102. Some or all of consumption usage data 521 can include training usage data 522, corresponding to usage in conjunction with a certification program or other user training provided by one or more subsystems. The training usage data 522 can correspond to each instance where diagnosis feedback data was provided by user for a medical scan with known diagnosis data, but diagnosis feedback data is not utilized by a subsystem to generate, edit, and/or confirm diagnosis data 440 of the medical scan, as it is instead utilized to train a user and/or determine performance data for a user.

Usage data 520 can include contribution usage data 523, which can include a listing of, or aggregate data associated with, usages of one or more subsystems 101 by the user, for example, where the user is generating and/or otherwise providing data and/or feedback that can is utilized by the subsystems, for example, to generate, edit, and/or confirm diagnosis data 440 and/or to otherwise populate, modify, or confirm portions of the medical scan database or other subsystem data. For example, the contribution usage data 523 can correspond to diagnosis feedback data received from user, used to generate, edit, and/or confirm diagnosis data. The contribution usage data 523 can include interactive interface feature data 524 corresponding to the interactive interface features utilized with respect to the contribution.

The consumption usage data 521 and/or the contribution usage data 523 can include medical scan entry 352 whose entries the user utilized and/or contributed to, can indicate one or more specific attributes of a medical scan entry 352 that a user utilized and/or contributed to, and/or a log of the user input generated by a client device of the user in conjunction with the data usage. The contribution usage data 523 can include the diagnosis data that the user may have generated and/or reviewed, for example, indicated by, mapped to, and/or used to generate the annotation author data 450 of corresponding medical scan entries 352. Some usages may correspond to both consumption usage of the consumption usage data 521 and contribution usage of the contribution usage data 523. The usage data 520 can also indicate one or more subsystems 101 that correspond to each consumption and/or contribution.

The user profile entry can include performance score data 530. This can include one or more performance scores generated based on the contribution usage data 523 and/or training usage data 522. The performance scores can include separate performance scores generated for every contribution in the contribution usage data 523 and/or training usage data 522 and/or generated for every training consumption usages corresponding to a training program. As used herein, a “high” performance score refers to a more favorable performance or rating than a “low” performance score.

The performance score data can include accuracy score data 531, which can be generated automatically by a subsystem for each contribution, for example, based on comparing diagnosis data received from a user to data to known truth data such as medical scans with a truth flag 461, for example, retrieved from the corresponding medical scan entry 352 and/or based on other data corresponding to the medical scan, for example, received from an expert user that later reviewed the contribution usage data of the user and/or generated automatically by a subsystem. The accuracy score data 531 can include an aggregate accuracy score generated automatically by a subsystem, for example, based on the accuracy data of multiple contributions by the user over time.

The performance data can also include efficiency score data 532 generated automatically by a subsystem for each contribution based on an amount of time taken to complete a contribution, for example, from a time the request for a contribution was sent to the client device to a time that the contribution was received from the client device, based on timing data received from the client device itself, and/or based on other factors. The efficiency score can include an aggregate efficiency score, which can be generated automatically by a subsystem based on the individual efficiency scores over time and/or based on determining a contribution completion rate, for example based on determining how many contributions were completed in a fixed time window.

Aggregate performance score data 533 can be generated automatically by a subsystem based on the aggregate efficiency and/or accuracy data. The aggregate performance data can include categorized performance data 534, for example, corresponding to different scan types, different anatomical regions, different subsystems, different interactive interface features and/or display parameters. The categorized performance data 534 can be determined automatically by a subsystem based on the scan type data 421 and/or anatomical region data 422 of the medical scan associated with each contribution, one or more subsystems 101 associated with each contribution, and/or interactive interface feature data 524 associated with each contribution. The aggregate performance data can also be based on performance score data 530 of individual employees if the user corresponds to a medical entity, for example, retrieved based on user profile identifiers 355 included in the employee data 513. The performance score data can also include ranking data 535, which can include an overall ranking or categorized rankings, for example, generated automatically by a subsystem or the database itself based on the aggregate performance data.

In some embodiments, aggregate data for each user can be further broken down based on scores for distinct scan categories, for example, based on the scan classifier data 420, for example, where a first aggregate data score is generated for a user “A” based on scores from all knee x-rays, and a second aggregate data score is generated for user A based on scores from all chest CT scans. Aggregate data for each user can be further based on scores for distinct diagnosis categories, where a first aggregate data score is generated for user A based on scores from all normal scans, and a second aggregate data score is generated for user A based on scores from all scans that contain an abnormality. This can be further broken down, where a first aggregate score is generated for user A based on all scores from scans that contain an abnormality of a first type and/or in a first anatomical location, and a second aggregate score is generated for A based on all scores from scans that contain an abnormality of a second type and/or in a second location. Aggregate data for each user can be further based on affiliation data, where a ranking is generated for a medical professional “B” based on scores from all medical professionals with the same affiliation data, and/or where a ranking is generated for a hospital “C” based on scores for all hospitals, all hospitals in the same geographical region, etc. Aggregate data for each user can be further based on scores for interface features, where a first aggregate data score is generated for user A based on scores using a first interface feature, and a second aggregate data score is generated for user A based on scores using a first interface feature.

The user profile entry can include qualification data 540. The qualification data can include experience data 541 such as education data, professional practice data, number of years practicing, awards received, etc. The qualification data 540 can also include certification data 542 corresponding to certifications earned based on contributions to one or more subsystems, for example, assigned to users automatically by a subsystem based on the performance score data 530 and/or based on a number of contributions in the contribution usage data 523 and/or training usage data 522. For example, the certifications can correspond to standard and/or recognized certifications to train medical professionals and/or incentivize medical professionals to use the system. The qualification data 540 can include expert data 543. The expert data 543 can include a binary expert identifier, which can be generated automatically by a subsystem based on experience data 541, certification data 542, and/or the performance score data 530, and can indicate whether the user is an expert user. The expert data 543 can include a plurality of categorized binary expert identifiers corresponding to a plurality of qualification categories corresponding to corresponding to scan types, anatomical regions, and/or the particular subsystems. The categorized binary expert identifiers can be generated automatically by a subsystem based on the categorized performance data 534 and/or the experience data 541. The categories be ranked by performance score in each category to indicate particular specialties. The expert data 543 can also include an expert ranking or categorized expert ranking with respect to all experts in the system.

The user profile entry can include subscription data 550, which can include a selected one of a plurality of subscription options that the user has subscribed to. For example, the subscription options can correspond to allowed usage of one or more subsystems, such as a number of times a user can utilize a subsystem in a month, and/or to a certification program, for example paid for by a user to receive training to earn a subsystem certification of certification data 542. The subscription data can include subscription expiration information, and/or billing information. The subscription data can also include subscription status data 551, which can for example indicate a number of remaining usages of a system and/or available credit information. For example, the remaining number of usages can decrease and/or available credit can decrease in response to usages that utilize one or more subsystems as a service, for example, indicated in the consumption usage data 521 and/or training usage data 522. In some embodiments, the remaining number of usages can increase and/or available credit can increase in response to usages that correspond to contributions, for example, based on the contribution usage data 523. An increase in credit can be variable, and can be based on a determined quality of each contribution, for example, based on the performance score data 530 corresponding to the contribution where a higher performance score corresponds to a higher increase in credit, based on scan priority data 427 of the medical scan where contributing to higher priority scans corresponds to a higher increase in credit, or based on other factors.

The user profile entry 354 can include interface preference data 560. The interface preference data can include a preferred interactive interface feature set 561, which can include one or more interactive interface feature identifiers and/or one or more interactive interface version identifiers of interface feature entries 358 and/or version identifiers of the interface features. Some or all of the interface features of the preferred interactive interface feature set 561 can correspond to display parameter data 470 of medical scans. The preferred interactive interface feature set 561 can include a single interactive feature identifier for one or more feature types and/or interface types, and/or can include a single interactive interface version identifier for one or more interface categories. The preferred interactive interface feature set 561 can include a ranking of multiple features for the same feature type and/or interface type. The ranked and/or unranked preferred interactive interface feature set 561 can be generated based on user input to an interactive interface of the client device to select and/or rank some or all of the interface features and/or versions. Some or all of the features and/or versions of the preferred interactive feature set can be selected and/or ranked automatically by a subsystem such as the medical scan interface evaluator system, for example based on interface feature performance score data and/or feature popularity data. Alternatively or in addition, the performance score data 530 can be utilized by a subsystem to automatically determine the preferred interactive feature set, for example, based on the scores in different feature-based categories of the categorized performance data 534.

The user profile entry 354 can include report formatting data 570, which can indicate report formatting preferences indicated by the user. This can include font, text size, header data, bulleting or numbering type, margins, file type, preferences for including one or more full or cropped image slices 412, preferences for including similar medical scans, preferences for including additional medical scans in reports, or other formatting preference to list natural language text data and/or image data corresponding to each abnormality. Some or all of the report formatting data 570 can be based on interface preference data 560. The report formatting data 570 can be used by one or more subsystems to automatically generate report data 449 of medical scans based on the preferences of the requesting user.

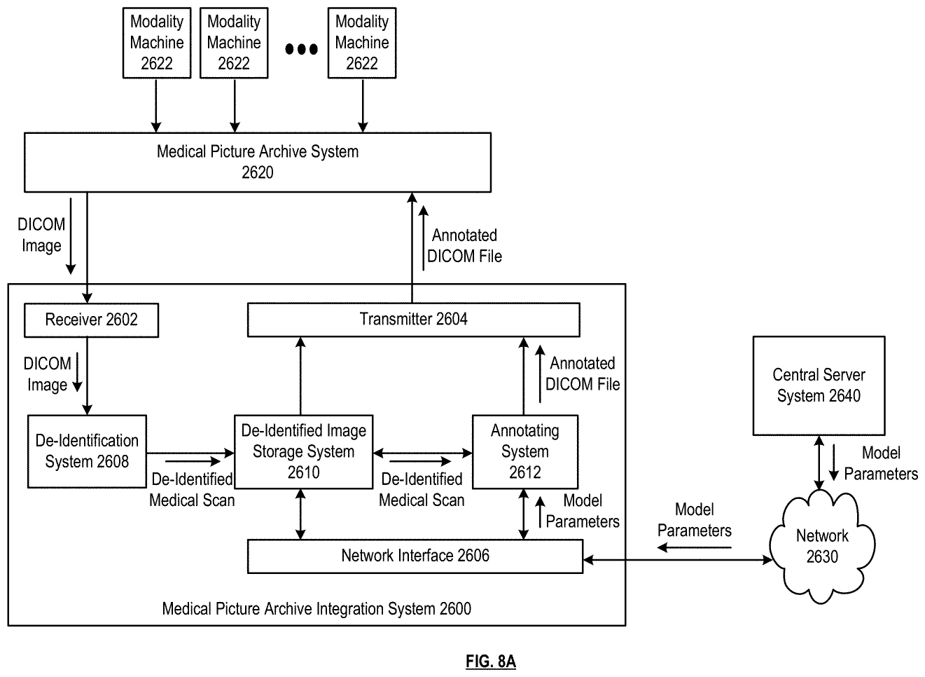

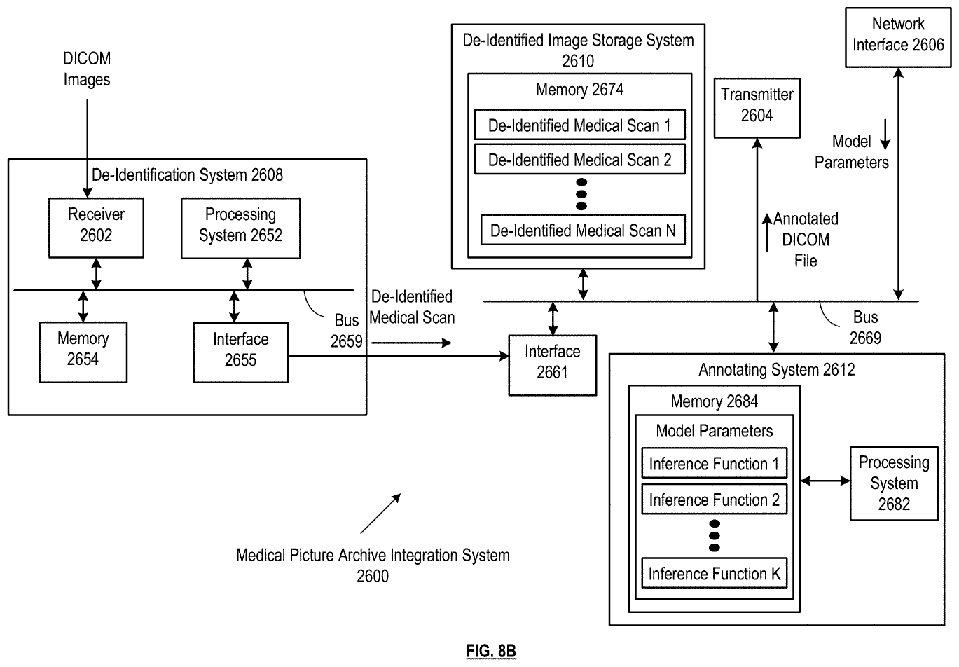

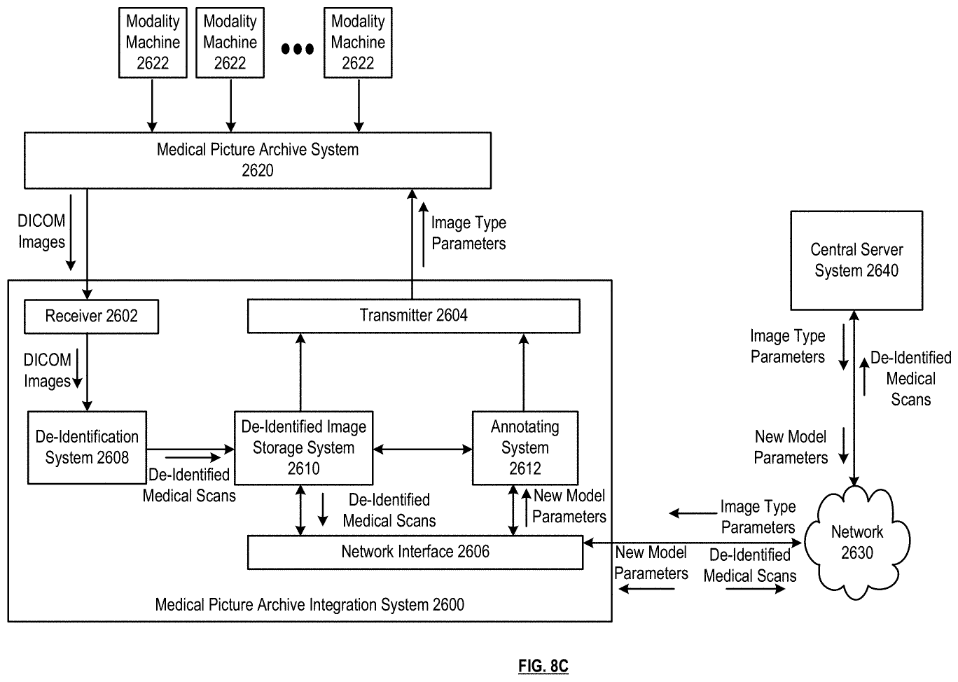

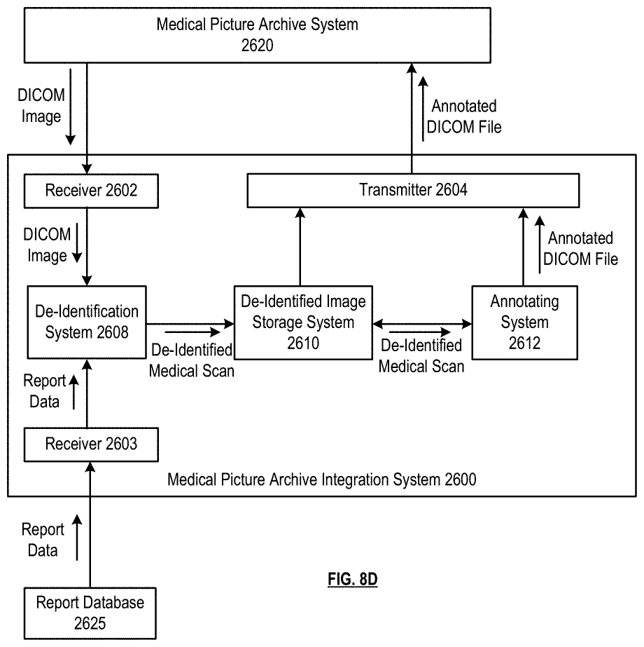

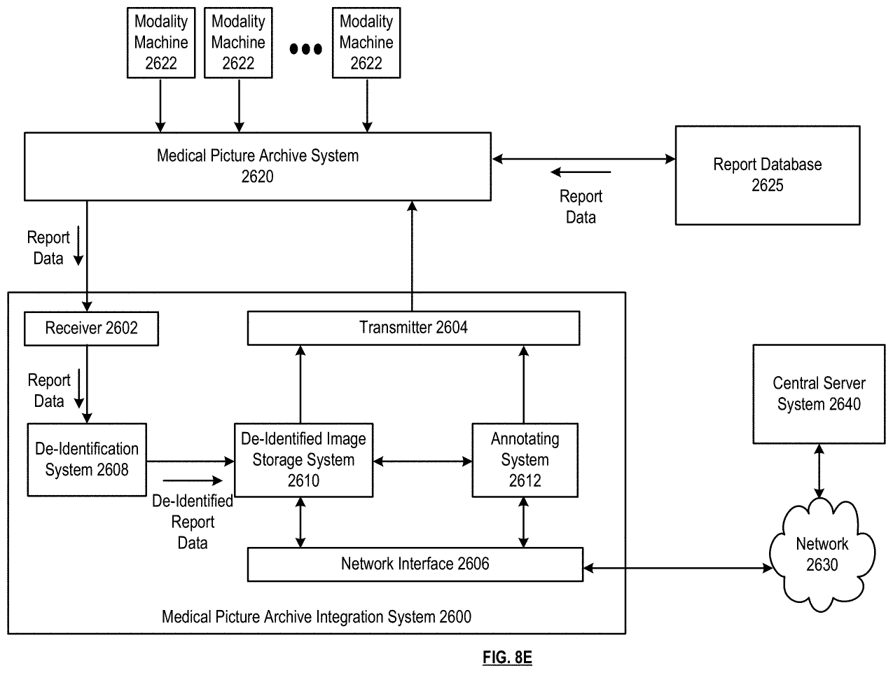

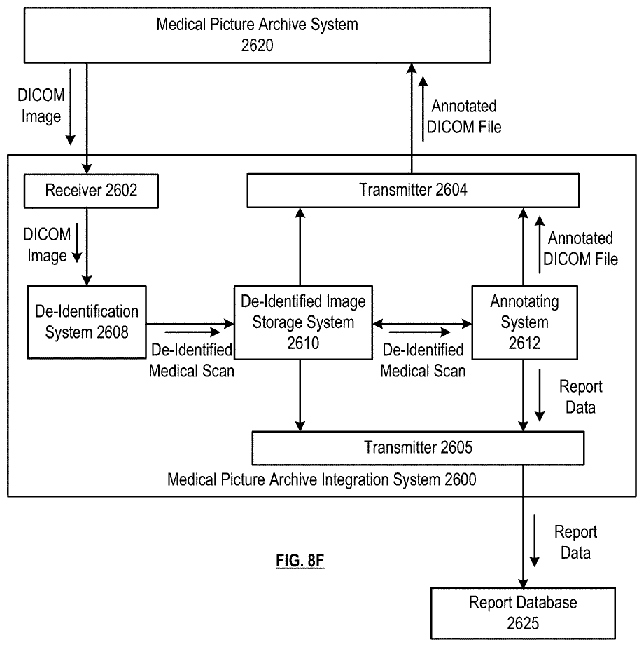

FIG. 5B presents an embodiment of a medical scan analysis function entry 356, stored in medical scan analysis function database 346 or otherwise associated with one of a plurality of medical scan analysis functions trained by and/or utilized by one or more subsystems 101. For example, a medical scan analysis function can include one or more medical scan image analysis functions trained by the medical scan image analysis system 112; one or more medical scan natural language analysis functions trained by the medical scan natural language analysis system 114; one or more medical scan similarity analysis function trained by the medical scan image analysis system 112, the medical scan natural language analysis system 114, and/or the medical scan comparison system 116; one or more medical report generator functions trained by the medical scan natural language analysis system 114 and/or the medical scan image analysis system 112, and/or the medical report analysis function trained by the medical scan natural language analysis system 114. Some or all of the medical scan analysis functions can correspond to medical scan inference functions of the medical scan diagnosing system 108, the de-identification function and/or the inference functions utilized by a medical picture archive integration system as discussed in conjunction with FIGS. 8A-8F, or other functions and/or processes described herein in conjunction with one or more subsystems 101. Each medical scan analysis function entry 356 can include a medical scan analysis function identifier 357.

A medical scan analysis function entry 356 can include function classifier data 610. Function classifier data 610 can include input and output types corresponding to the function. For example the function classifier data can include input scan category 611 that indicates which types of scans can be used as input to the medical scan analysis function. For example, input scan category 611 can indicate that a medical scan analysis function is for chest CT scans from a particular hospital or other medical entity. The input scan category 611 can include one or more categories included in scan classifier data 420. In various embodiments, the input scan category 611 corresponds to the types of medical scans that were used to train the medical scan analysis function. Function classifier data 610 can also include output type data 612 that characterizes the type of output that will be produced by the function, for example, indicating that a medical scan analysis function is used to generate medical codes 447. The input scan category 611 can also include information identifying which subsystems 101 are responsible for running the medical scan analysis function.

A medical scan analysis function entry 356 can include training parameters 620. This can include training set data 621, which can include identifiers for the data used to train the medical scan analysis function, such as a set of medical scan identifiers 353 corresponding to the medical scans used to train the medical scan analysis function, a list of medical scan reports and corresponding medical codes used to train the medical scan analysis function, etc. Alternatively or in addition to identifying particular scans of the training set, the training set data 621 can identify training set criteria, such as necessary scan classifier data 420, necessary abnormality locations, classifiers, or other criteria corresponding to abnormality annotation data 442, necessary confidence score data 460, for example, indicating that only medical scans with diagnosis data 440 assigned a truth flag 461 or with confidence score data 460 otherwise comparing favorably to a training set confidence score threshold are included, a number of medical scans to be included and proportion data corresponding to different criteria, or other criteria used to populate a training set with data of medical scans. Training parameters 620 can include model type data 622 indicating one or more types of model, methods, and/or training functions used to determine the medical scan analysis function by utilizing the training set 621. Training parameters 620 can include model parameter data 623 that can include a set of features of the training data selected to train the medical scan analysis function, determined values for weights corresponding to selected input and output features, determined values for model parameters corresponding to the model itself, etc. The training parameter data can also include testing data 624, which can identify a test set of medical scans or other data used to test the medical scan analysis function. The test set can be a subset of training set 621, include completely separate data than training set 621, and/or overlap with training set 621. Alternatively or in addition, testing data 624 can include validation parameters such as a percentage of data that will be randomly or pseudo-randomly selected from the training set for testing, parameters characterizing a cross validation process, or other information regarding testing. Training parameters 620 can also include training error data 625 that indicates a training error associated with the medical scan analysis function, for example, based on applying cross validation indicated in testing data 624.

A medical scan analysis function entry 356 can include performance score data 630. Performance data can include model accuracy data 631, for example, generated and/or updated based on the accuracy of the function when performed on new data. For example, the model accuracy data 631 can include or be calculated based on the model error for determined for individual uses, for example, generated by comparing the output of the medical scan analysis function to corresponding data generated by user input to interactive interface 275 in conjunction with a subsystem 101 and/or generated by comparing the output of the medical scan analysis function to medical scans with a truth flag 461. The model accuracy data 631 can include aggregate model accuracy data computed based on model error of individual uses of the function over time. The performance score data 630 can also include model efficiency data 632, which can be generated based on how quickly the medical scan analysis function performs, how much memory is utilized by medical scan analysis function, or other efficiency data relating to the medical scan analysis function. Some or all of the performance score data 630 can be based on training error data 625 or other accuracy and/or efficiency data determined during training and/or validation. As used herein, a “high” performance score refers to a more favorable performance or rating than a “low” performance score.

A medical scan analysis function entry 356 can include version data 640. The version data can include a version identifier 641. The version data can indicate one or more previous version identifiers 642, which can map to version identifiers 641 stored in other medical scan analysis function entry 356 that correspond to previous versions of the function. Alternatively or in addition, the version data can indicate multiple versions of the same type based on function classifier data 610, can indicate the corresponding order and/or rank of the versions, and/or can indicate training parameters 620 associated with each version.

A medical scan analysis function entry 356 can include remediation data 650. Remediation data 650 can include remediation instruction data 651 which can indicate the steps in a remediation process indicating how a medical scan analysis function is taken out of commission and/or reverted to a previous version in the case that remediation is necessary. The version data 640 can further include remediation criteria data 652, which can include threshold data or other criteria used to automatically determine when remediation is necessary. For example, the remediation criteria data 652 can indicate that remediation is necessary at any time where the model accuracy data and/or the model efficiency data compares unfavorably to an indicated model accuracy threshold and/or indicated model efficiency threshold. The remediation data 650 can also include recommissioning instruction data 653, identifying required criteria for recommissioning a medical scan analysis function and/or updating a medical scan analysis function. The remediation data 650 can also include remediation history, indicating one or more instances that the medical scan analysis function was taken out of commission and/or was recommissioned.

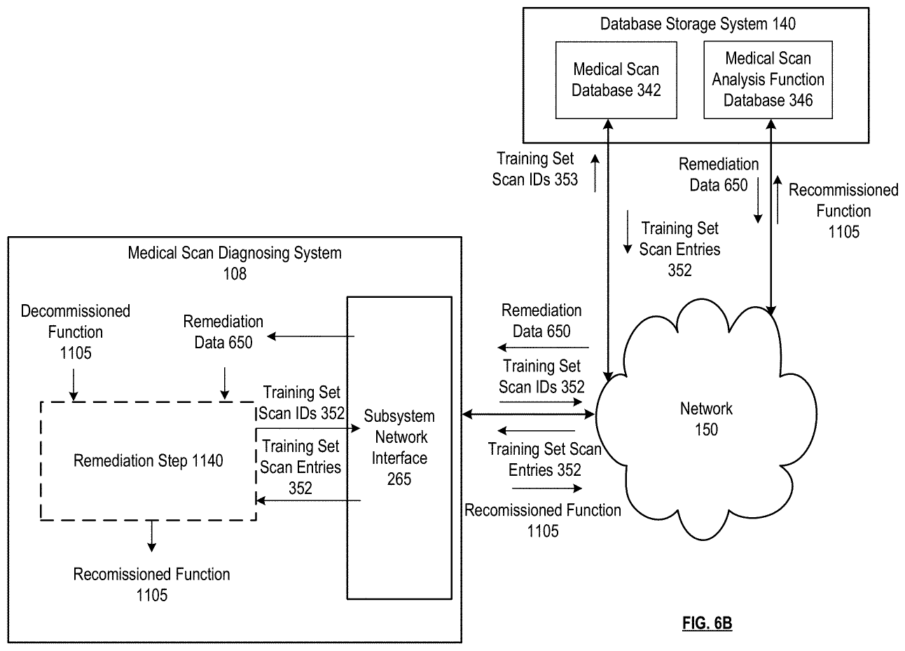

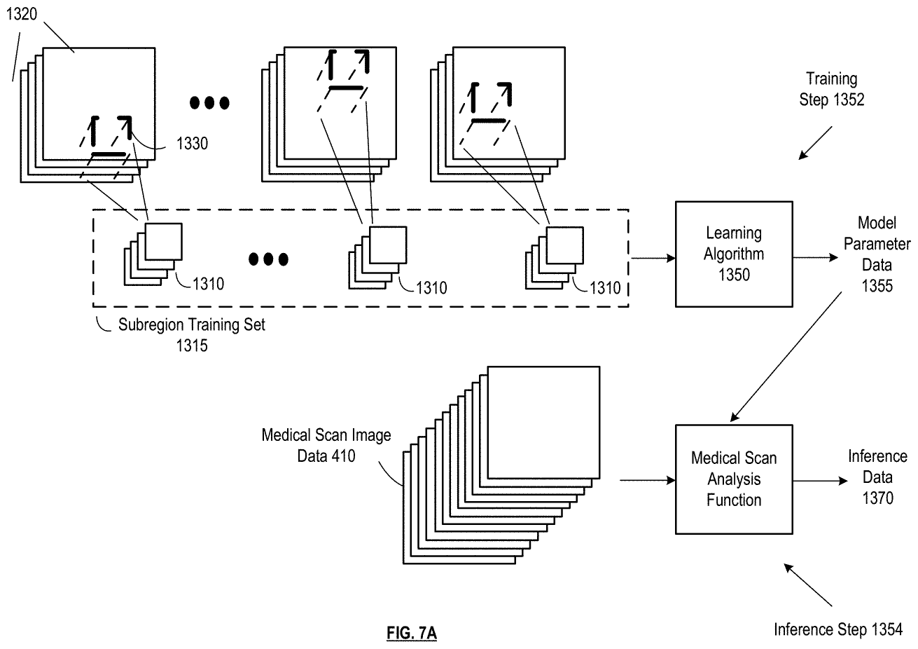

FIGS. 6A and 6B present an embodiment of a medical scan diagnosing system 108. The medical scan diagnosing system 108 can generate inference data 1110 for medical scans by utilizing a set of medical scan inference functions 1105, stored and run locally, stored and run by another subsystem 101, and/or stored in the medical scan analysis function database 346, where the function and/or parameters of the function can be retrieved from the database by the medical scan diagnosing system. For example, the set of medical scan inference function 1105 can include some or all medical scan analysis functions described herein or other functions that generate inference data 1110 based on some or all data corresponding to a medical scan such as some or all data of a medical scan entry 352. Each medical scan inference function 1105 in the set can correspond to a scan category 1120, and can be trained on a set of medical scans that compare favorably to the scan category 1120. For example, each inference function can be trained on a set of medical scans of the one or more same scan classifier data 420, such as the same and/or similar scan types, same and/or similar anatomical regions locations, same and/or similar machine models, same and/or similar machine calibration, same and/or similar contrasting agent used, same and/or similar originating entity, same and/or similar geographical region, and/or other classifiers. Thus, the scan categories 1120 can correspond to one or more of a scan type, scan anatomical region data, hospital or other originating entity data, machine model data, machine calibration data, contrast agent data, geographic region data, and/or other scan classifying data 420. For example, a first medical scan inference function can be directed to characterizing knee x-rays, and a second medical scan inference function can be directed to chest CT scans. As another example, a first medical scan inference function can be directed to characterizing CT scans from a first hospital, and a second medical scan image analysis function can be directed to characterizing CT scans from a second hospital.

Training on these categorized sets separately can ensure each medical scan inference function 1105 is calibrated according to its scan category 1120, for example, allowing different inference functions to be calibrated on type specific, anatomical region specific, hospital specific, machine model specific, and/or region-specific tendencies and/or discrepancies. Some or all of the medical scan inference functions 1105 can be trained by the medical scan image analysis system and/or the medical scan natural language processing system, and/or some medical scan inference functions 1105 can utilize both image analysis and natural language analysis techniques to generate inference data 1110. For example, some or all of the inference functions can utilize image analysis of the medical scan image data 410 and/or natural language data extracted from abnormality annotation data 442 and/or report data 449 as input, and generate diagnosis data 440 such as medical codes 447 as output. Each medical scan inference function can utilize the same or different learning models to train on the same or different features of the medical scan data, with the same or different model parameters, for example indicated in the model type data 622 and model parameter data 623. Model type and/or parameters can be selected for a particular medical scan inference function based on particular characteristics of the one or more corresponding scan categories 1120, and some or all of the indicated in the model type data 622 and model parameter data 623 can be selected automatically by a subsystem during the training process based on the particular learned and/or otherwise determined characteristics of the one or more corresponding scan categories 1120.

As shown in FIG. 6A, the medical scan diagnosing system 108 can automatically select a medical scan for processing in response to receiving it from a medical entity via the network. Alternatively, the medical scan diagnosing system 108 can automatically retrieve a medical scan from the medical scan database that is selected based on a request received from a user for a particular scan and/or based on a queue of scans automatically ordered by the medical scan diagnosing system 108 or another subsystem based on scan priority data 427.

Once a medical scan to be processed is determined, the medical scan diagnosing system 108 can automatically select an inference function 1105 based on a determined scan category 1120 of the selected medical scan and based on corresponding inference function scan categories. The scan category 1120 of a scan can be determined based one some or all of the scan classifier data 420 and/or based on other metadata associated with the scan. This can include determining which one of the plurality of medical scan inference functions 1105 matches or otherwise compares favorably to the scan category 1120, for example, by comparing the scan category 1120 to the input scan category of the function classifier data 610.

Alternatively or in addition, the medical scan diagnosing system 108 can automatically determine which medical scan inference function 1105 is utilized based on an output preference that corresponding to a desired type of inference data 1110 that is outputted by an inference function 1105. The output preference designated by a user of the medical scan diagnosing system 108 and/or based on the function of a subsystem 101 utilizing the medical scan diagnosing system 108. For example, the set of inference functions 1105 can include inference functions that are utilized to indicate whether or not a medical scan is normal, to automatically identify at least one abnormality in the scan, to automatically characterize the at least one abnormality in the scan, to assign one or more medical codes to the scan, to generate natural language text data and/or a formatted report for the scan, and/or to automatically generate other diagnosis data such as some or all of diagnosis data 440 based on the medical scan. Alternatively or in addition, some inference functions can also be utilized to automatically generate confidence score data 460, display parameter data 470, and/or similar scan data 480. The medical scan diagnosing system 108 can compare the output preference to the output type data 612 of the medical scan inference function 1105 to determine the selected inference function 1105. For example, this can be used to decide between a first medical scan inference function that automatically generates medical codes and a second medical scan inference function that automatically generates natural language text for medical reports based on the desired type of inference data 1110.

Prior to performing the selected medical scan inference function 1105, the medical scan diagnosing system 108 can automatically perform an input quality assurance function 1106 to ensure the scan classifier data 420 or other metadata of the medical scan accurately classifies the medical scan such that the appropriate medical scan inference function 1105 of the appropriate scan category 1120 is selected. The input quality assurance function can be trained on, for example, medical scan image data 410 of plurality of previous medical scans with verified scan categories. Thus, the input quality assurance function 1106 can take medical scan image data 410 as input and can generate an inferred scan category as output. The inferred scan category can be compared to the scan category 1120 of the scan, and the input quality assurance function 1106 can determine whether or not the scan category 1120 is appropriate by determining whether the scan category 1120 compares favorably to the automatically generated inferred scan category. The input quality assurance function 1106 can also be utilized to reassign the generated inferred scan category to the scan category 1120 when the scan category 1120 compares favorably to the automatically generated inferred scan category. The input quality assurance function 1106 can also be utilized to assign the generated inferred scan category to the scan category 1120 for incoming medical scans that do not include any classifying data, and/or to add classifiers in scan classifier data 420 to medical scans missing one or more classifiers.

In various embodiments, upon utilizing the input quality assurance function 1106 to determine that the scan category 1120 determined by a scan classifier data 420 or other metadata is inaccurate, the medical scan diagnosing system 108 can transmit an alert and/or an automatically generated inferred scan category to the medical entity indicating that the scan is incorrectly classified in the scan classifier data 420 or other metadata. In some embodiments, the medical scan diagnosing system 108 can automatically update performance score data corresponding to the originating entity of the scan indicated in originating entity data 423, or another user or entity responsible for classifying the scan, for example, where a lower performance score is generated in response to determining that the scan was incorrectly classified and/or where a higher performance score is generated in response to determining that the scan was correctly classified.

In some embodiments, the medical scan diagnosing system 108 can transmit the medical scan and/or the automatically generated inferred scan category to a selected user. The user can be presented the medical scan image data 410 and/or other data of the medical scan via the interactive interface 275, for example, displayed in conjunction with the medical scan assisted review system 102. The interface can prompt the user to indicate the appropriate scan category 1120 and/or prompt the user to confirm and/or edit the inferred scan category, also presented to the user. For example, scan review data can be automatically generated to reflect the user generated and/or verified scan category 1120. This user indicated scan category 1120 can be utilized to select to the medical scan inference function 1105 and/or to update the scan classifier data 420 or other metadata accordingly. In some embodiments, for example, where the scan review data indicates that the selected user disagrees with the automatically generated inferred scan category created by the input quality assurance function 1106, the medical scan diagnosing system 108 can automatically update performance score data 630 of the input quality assurance function 1106 by generating a low performance score and/or determine to enter the remediation step 1140 for the input quality assurance function 1106.

The medical scan diagnosing system 108 can also automatically perform an output quality assurance step after a medical scan inference function 1105 has been performed on a medical scan to produce the inference data 1110, as illustrated in the embodiment presented in FIG. 6B. The output quality assurance step can be utilized to ensure that the selected medical scan inference function 1105 generated appropriate inference data 1110 based on expert feedback. The inference data 1110 generated by performing the selected medical scan inference function 1105 can be sent to a client device 120 of a selected expert user, such as an expert user in the user database selected based on categorized performance data and/or qualification data that corresponds to the scan category 1120 and/or the inference itself, for example, by selecting an expert user best suited to review an identified abnormality classifier category 444 and/or abnormality pattern category 446 in the inference data 1110 based on categorized performance data and/or qualification data of a corresponding user entry. The selected user can also correspond to a medical professional or other user employed at the originating entity and/or corresponding to the originating medical professional, indicated in the originating entity data 423.

FIG. 6B illustrates an embodiment of the medical scan diagnosing system 108 in conjunction with performing a remediation step 1140. The medical scan diagnosing system 108 can monitor the performance of the set of medical scan inference functions 1105, for example, based on evaluating inference accuracy data outputted by an inference data evaluation function and/or based monitoring on the performance score data 630 in the medical scan analysis function database, and can determine whether or not if the corresponding medical scan inference function 1105 is performing properly. This can include, for example, determining if a remediation step 1140 is necessary for a medical scan inference function 1105, for example, by comparing the performance score data 630 and/or inference accuracy data to remediation criteria data 652. Determining if a remediation step 1140 is necessary can also be based on receiving an indication from the expert user or another user that remediation is necessary for one or more identified medical scan inference functions 1105 and/or for all of the medical scan inference functions 1105.

In various embodiments, a remediation evaluation function is utilized to determine if a remediation step 1140 is necessary for medical scan inference function 1105. The remediation evaluation function can include determining that remediation is necessary when recent accuracy data and/or efficiency data of a particular medical scan inference function 1105 is below the normal performance level of the particular inference function. The remediation evaluation function can include determining that remediation is necessary when recent or overall accuracy data and/or efficiency data of a particular medical scan inference function 1105 is below a recent or overall average for all or similar medical scan inference functions 1105. The remediation evaluation function can include determining that remediation is necessary only after a threshold number of incorrect diagnoses are made. In various embodiments, multiple threshold number of incorrect diagnoses correspond to different diagnoses categories. For example, the threshold number of incorrect diagnoses for remediation can be higher for false negative diagnoses than false positive diagnoses. Similarly, categories corresponding to different diagnosis severities and/or rarities can have different thresholds, for example where a threshold number of more severe and/or more rare diagnoses that were inaccurate to necessitate remediation is lower than a threshold number of less severe and/or less rare diagnoses that were inaccurate.

The remediation step 1140 can include automatically updating an identified medical inference function 1105. This can include automatically retraining identified medical inference function 1105 on the same training set or on a new training set that includes new data, data with higher corresponding confidence scores, or data selected based on new training set criteria. The identified medical inference function 1105 can also be updated and/or changed based on the review data received from the client device. For example, the medical scan and expert feedback data can be added to the training set of the medical scan inference function 1105, and the medical scan inference function 1105 can be retrained on the updated training set. Alternatively or in addition, the expert user can identify additional parameters and/or rules in the expert feedback data based on the errors made by the inference function in generating the inference data 1110 for the medical scan, and these parameters and/or rules can be applied to update the medical scan inference function, for example, by updating the model type data 622 and/or model parameter data 623.

The remediation step 1140 can also include determining to split a scan category 1120 into two or more subcategories. Thus, two or more new medical scan inference functions 1105 can be created, where each new medical scan inference functions 1105 is trained on a corresponding training set that is a subset of the original training set and/or includes new medical scan data corresponding to the subcategory. This can allow medical scan inference functions 1105 to become more specialized and/or allow functions to utilize characteristics and/or discrepancies specific to the subcategory when generating inference data 1110. Similarly, a new scan category 1120 that was not previously represented by any of the medical scan inference functions 1105 can be added in the remediation step, and a new medical scan inference functions 1105 can be trained on a new set of medical scan data that corresponds to the new scan category 1120. Splitting a scan category and/or adding a scan category can be determined automatically by the medical scan diagnosing system 108 when performing the remediation step 1140, for example, based on performance score data 630. This can also be determined based on receiving instructions to split a category and/or add a new scan category from the expert user or other user of the system.

After a medical scan inference function 1105 is updated or created for the first time, the remediation step 1140 can further undergo a commissioning test, which can include rigorous testing of the medical scan inference function 1105 on a testing set, for example, based on the training parameters 620. For example, the commissioning test can be passed when the medical scan inference function 1105 generates a threshold number of correct inference data 1110 and/or the test can be passed if an overall or average discrepancy level between the inference data and the test data is below a set error threshold. The commissioning test can also evaluate efficiency, where the medical scan inference function 1105 only passes the commissioning test if it performs at or exceeds a threshold efficiency level. If the medical scan inference function 1105 fails the commissioning test, the model type and/or model parameters can be modified automatically or based on user input, and the medical scan inference function can be retested, continuing this process until the medical scan inference function 1105 passes the commissioning test.

The remediation step 1140 can include decommissioning the medical scan inference function 1105, for example, while the medical scan inference function is being retrained and/or is undergoing the commissioning test. Incoming scans to the medical scan diagnosing system 108 with a scan category 1120 corresponding to a decommissioned medical scan inference function 1105 can be sent directly to review by one or more users, for example, in conjunction with the medical scan annotator system 106. These user-reviewed medical scans and corresponding annotations can be included in an updated training set used to train the decommissioned medical scan inference function 1105 as part of the remediation step 1140. In some embodiments, previous versions of the plurality of medical scan image analysis functions can be stored in memory of the medical scan diagnosing system and/or can be determined based on the version data 640 of a medical scan inference function 1105. A previous version of a medical scan inference function 1105, such as most recent version or version with the highest performance score, can be utilized during the remediation step 1140 as an alternative to sending all medical scans to user review.

A medical scan inference function can also undergo the remediation step 1140 automatically in response to a hardware and/or software update on processing, memory, and/or other computing devices where the medical scan inference function 1105 is stored and/or performed. Different medical scan inference functions 1105 can be containerized on their own devices by utilizing a micro-service architecture, so hardware and/or software updates may only necessitate that one of the medical scan inference functions 1105 undergo the remediation step 1140 while the others remain unaffected. A medical scan inference function 1105 can also undergo the remediation step 1140 automatically in response to normal system boot-up, and/or periodically in fixed intervals. For example, in response to a scheduled or automatically detected hardware and/or software update, change, or issue, one or more medical scan inference functions 1105 affected by this hardware or software can be taken out of commission until they each pass the commissioning test. Such criteria can be indicated in the remediation criteria data 652.

The medical scan diagnosing system 108 can automatically manage usage data, subscription data, and/or billing data for the plurality of users corresponding to user usage of the system, for example, by utilizing, generating, and/or updating some or all of the subscription data of the user database. Users can pay for subscriptions to the system, which can include different subscription levels that can correspond to different costs. For example, a hospital can pay a monthly cost to automatically diagnose up to 100 medical scans per month. The hospital can choose to upgrade their subscription or pay per-scan costs for automatic diagnosing of additional scans received after the quota is reached and/or the medical scan diagnosing system 108 can automatically send medical scans received after the quota is reached to an expert user associated with the hospital. In various embodiments incentive programs can be used by the medical scan diagnosing system to encourage experts to review medical scans from different medical entities. For example, an expert can receive credit to their account and/or subscription upgrades for every medical scan reviewed, or after a threshold number of medical scans are reviewed. The incentive programs can include interactions by a user with other subsystems, for example, based on contributions made to medical scan entries via interaction with other subsystems.

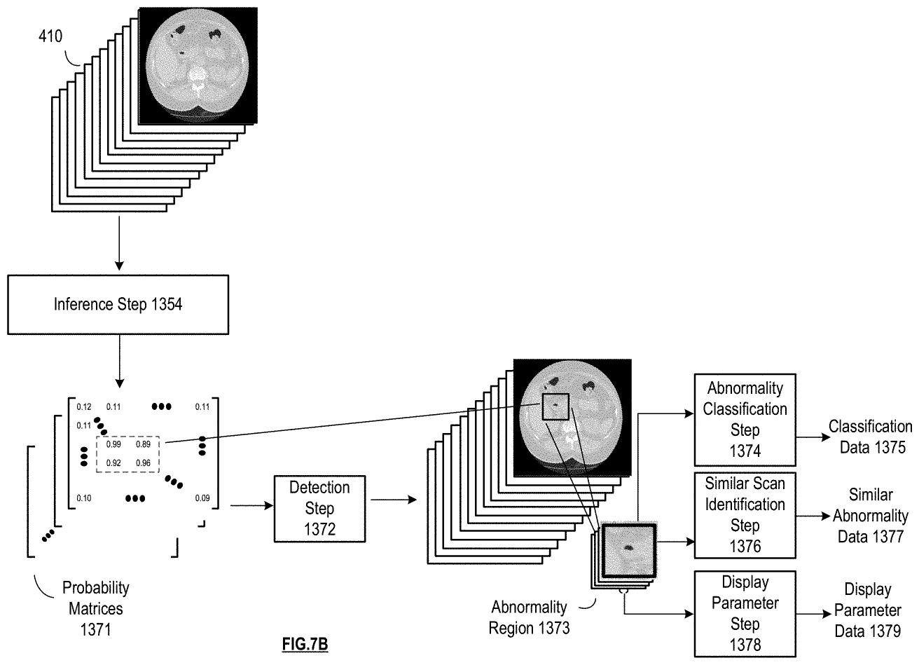

FIG. 7A presents an embodiment of a medical scan image analysis system 112. A training set of medical scans used to train one more medical scan image analysis functions can be received from one or more client devices via the network and/or can be retrieved from the medical scan database 342, for example, based on training set data 621 corresponding to medical scan image analysis functions. Training set criteria, for example, identified in training parameters 620 of the medical scan image analysis function, can be utilized to automatically identify and select medical scans to be included in the training set from a plurality of available medical scans. The training set criteria can be automatically generated based on, for example, previously learned criteria, and/or training set criteria can be received via the network, for example, from an administrator of the medical scan image analysis system. The training set criteria can include a minimum training set size. The training set criteria can include data integrity requirements for medical scans in the training set such as requiring that the medical scan is assigned a truth flag 461, requiring that performance score data for a hospital and/or medical professional associated with the medical scan compares favorably to a performance score threshold, requiring that the medical scan has been reviewed by at least a threshold number of medical professionals, requiring that the medical scan and/or a diagnosis corresponding to a patient file of the medical scan is older than a threshold elapsed time period, or based on other criteria intended to insure that the medical scans and associated data in the training set is reliable enough to be considered “truth” data. The training set criteria can include longitudinal requirements such the number of required subsequent medical scans for the patient, multiple required types of additional scans for the patient, and/or other patient file requirements.

The training set criteria can include quota and/or proportion requirements for one or more medical scan classification data. For example, the training set criteria can include meeting quota and/or proportion requirements for one or more scan types and/or human body location of scans, meeting quota or proportion requirements for a number of normal medical scans and a number of medicals scans with identified abnormalities, meeting quota and/or proportion requirements for a number of medical scans with abnormalities in certain locations and/or a number of medical scans with abnormalities that meet certain size, type, or other characteristics, meeting quota and/or proportion data for a number of medical scans with certain diagnosis or certain corresponding medical codes, and/or meeting other identified quota and/or proportion data relating to metadata, patient data, or other data associated with the medical scans.

In some embodiments, multiple training sets are created to generate corresponding medical scan image analysis functions, for example, corresponding to some or all of the set of medical scan inference functions 1105. Some or all training sets can be categorized based on some or all of the scan classifier data 420 as described in conjunction with the medical scan diagnosing system 108, where medical scans are included in a training set based on their scan classifier data 420 matching the scan category of the training set. In some embodiments, the input quality assurance function 1106 or another input check step can be performed on medical scans selected for each training set to confirm that their corresponding scan classifier data 420 is correct. In some embodiments, the input quality assurance function can correspond to its own medical scan image analysis function, trained by the medical scan image analysis system, where the input quality assurance function utilizes high level computer vision technology to determine a scan category 1120 and/or to confirm the scan classifier data 420 already assigned to the medical scan.

In some embodiments, the training set will be used to create a single neural network model, or other model corresponding to model type data 622 and/or model parameter data 623 of the medical scan image analysis function that can be trained on some or all of the medical scan classification data described above and/or other metadata, patient data, or other data associated with the medical scans. In other embodiments, a plurality of training sets will be created to generate a plurality of corresponding neural network models, where the multiple training sets are divided based on some or all of the medical scan classification data described above and/or other metadata, patient data, or other data associated with the medical scans. Each of the plurality of neural network models can be generated based on the same or different learning algorithm that utilizes the same or different features of the medical scans in the corresponding one of the plurality of training sets. The medical scan classifications selected to segregate the medical scans into multiple training sets can be received via the network, for example based on input to an administrator client device from an administrator. The medical scan classifications selected to segregate the medical scans can be automatically determined by the medical scan image analysis system, for example, where an unsupervised clustering algorithm is applied to the original training set to determine appropriate medical scan classifications based on the output of the unsupervised clustering algorithm.

In embodiments where the medical scan image analysis system is used in conjunction with the medical scan diagnosing system, each of the medical scan image analysis functions associated with each neural network model can correspond to one of the plurality of neural network models generated by the medical scan image analysis system. For example, each of the plurality of neural network models can be trained on a training set classified on scan type, scan human body location, hospital or other originating entity data, machine model data, machine calibration data, contrast agent data, geographic region data, and/or other scan classifying data as discussed in conjunction with the medical scan diagnosing system. In embodiments where the training set classifiers are learned, the medical scan diagnosing system can determine which of the medical scan image analysis functions should be applied based on the learned classifying criteria used to segregate the original training set.

A computer vision-based learning algorithm used to create each neural network model can include selecting a three-dimensional subregion 1310 for each medical scan in the training set. This three-dimensional subregion 1310 can correspond to a region that is “sampled” from the entire scan that may represent a small fraction of the entire scan. Recall that a medical scan can include a plurality of ordered cross-sectional image slices. Selecting a three-dimensional subregion 1310 can be accomplished by selecting a proper image slice subset 1320 of the plurality of cross-sectional image slices from each of the plurality of medical scans, and by further selecting a two-dimensional subregion 1330 from each of the selected subset of cross-sectional image slices of the each of the medical scans. In some embodiments, the selected image slices can include one or more non-consecutive image slices and thus a plurality of disconnected three-dimensional subregions will be created. In other embodiments, the selected proper subset of the plurality of image slices correspond to a set of consecutive image slices, as to ensure that a single, connected three-dimensional subregion is selected. In some embodiments, entire scans of the training set are used to train the neural network model. In such embodiment, as used herein, the three-dimensional subregion 1310 can refer to all of the medical scan image data 410 of a medical scan.

In some embodiments, a density windowing step can be applied to the full scan or the selected three-dimensional subregion. The density windowing step can include utilizing a selected upper density value cut off and/or a selected lower density value cut off, and masking pixels with higher values than the upper density value cut off and/or masking pixels with lower values than the lower density value cut off. The upper density value cut off and/or a selected lower density value cut off can be determined based on based on the range and/or distribution of density values included in the region that includes the abnormality, and/or based on the range and/or distribution of density values associated with the abnormality itself, based on user input to a subsystem, based on display parameter data associated with the medical scan or associated with medical scans of the same type, and/or can be learned in the training step. In some embodiments, a non-linear density windowing function can be applied to alter the pixel density values, for example, to stretch or compress contrast. In some embodiments, this density windowing step can be performed as a data augmenting step, to create additional training data for a medical scan in accordance with different density windows.

Having determined the subregion training set 1315 of three-dimensional subregions 1310 corresponding to the set of full medical scans in the training set, the medical scan image analysis system can complete a training step 1352 by performing a learning algorithm on the plurality of three-dimensional subregions to generate model parameter data 1355 of a corresponding learning model. The learning model can include one or more of a neural network, an artificial neural network, a convolutional neural network, a Bayesian model, a support vector machine model, a cluster analysis model, or other supervised or unsupervised learning model. The model parameter data 1355 can generated by performing the learning algorithm 1350, and the model parameter data 1355 can be utilized to determine the corresponding medical scan image analysis functions. For example, some or all of the model parameter data 1355 can be mapped to the medical scan analysis function in the model parameter data 623 or can otherwise define the medical scan analysis function.

The training step 1352 can include creating feature vectors for each three-dimensional subregion of the training set for use by the learning algorithm 1350 to generate the model parameter data 1355. The feature vectors can include the pixel data of the three-dimensional subregions such as density values and/or grayscale values of each pixel based on a determined density window. The feature vectors can also include other features as additional input features or desired output features, such as known abnormality data such as location and/or classification data, patient history data such as risk factor data or previous medical scans, diagnosis data, responsible medical entity data, scan machinery model or calibration data, contrast agent data, medical code data, annotation data that can include raw or processed natural language text data, scan type and/or anatomical region data, or other data associated with the image, such as some or all data of a medical scan entry 352. Features can be selected based on administrator instructions received via the network and/or can be determined based on determining a feature set that reduces error in classifying error, for example, by performing a cross-validation step on multiple models created using different feature sets. The feature vector can be split into an input feature vector and output feature vector. The input feature vector can include data that will be available in subsequent medical scan input, which can include for example, the three-dimensional subregion pixel data and/or patient history data. The output feature vector can include data that will be inferred in in subsequent medical scan input and can include single output value, such as a binary value indicating whether or not the medical scan includes an abnormality or a value corresponding to one of a plurality of medical codes corresponding to the image. The output feature vector can also include multiple values which can include abnormality location and/or classification data, diagnosis data, or other output. The output feature vector can also include a determined upper density value cut off and/or lower density value cut off, for example, characterizing which pixel values were relevant to detecting and/or classifying an abnormality. Features included in the output feature vector can be selected to include features that are known in the training set, but may not be known in subsequent medical scans such as triaged scans to be diagnosed by the medical scan diagnosing system, and/or scans to be labeled by the medical scan report labeling system. The set of features in the input feature vector and output feature vector, as well as the importance of different features where each feature is assigned a corresponding weight, can also be designated in the model parameter data 1355.

Consider a medical scan image analysis function that utilizes a neural network. The neural network can include a plurality of layers, where each layer includes a plurality of neural nodes. Each node in one layer can have a connection to some or all nodes in the next layer, where each connection is defined by a weight value. Thus, the model parameter data 1355 can include a weight vector that includes weight values for every connection in the network. Alternatively or in addition, the model parameter data 1355 can include any vector or set of parameters associated with the neural network model, which can include an upper density value cut off and/or lower density value cut off used to mask some of the pixel data of an incoming image, kernel values, filter parameters, bias parameters, and/or parameters characterizing one or more of a plurality of convolution functions of the neural network model. The medical scan image analysis function can be utilized to produce the output vector as a function of the input feature vector and the model parameter data 1355 that characterizes the neural network model. In particular, the medical scan image analysis function can include performing a forward propagation step plurality of neural network layers to produce an inferred output vector based on the weight vector or other model parameter data 1355. Thus, the learning algorithm 1350 utilized in conjunction with a neural network model can include determining the model parameter data 1355 corresponding to the neural network model, for example, by populating the weight vector with optimal weights that best reduce output error.

In particular, determining the model parameter data 1355 can include utilizing a backpropagation strategy. The forward propagation algorithm can be performed on at least one input feature vector corresponding to at least one medical scan in the training set to propagate the at least one input feature vector through the plurality of neural network layers based on initial and/or default model parameter data 1355, such as an initial weight vector of initial weight values set by an administrator or chosen at random. The at least one output vector generated by performing the forward propagation algorithm on the at least one input feature vector can be compared to the corresponding at least one known output feature vector to determine an output error. Determining the output error can include, for example, computing a vector distance such as the Euclidian distance, or squared Euclidian distance, between the produced output vector and the known output vector, and/or determining an average output error such as an average Euclidian distance or squared Euclidian distance if multiple input feature vectors were employed. Next, gradient descent can be performed to determine an updated weight vector based on the output error or average output error. This gradient descent step can include computing partial derivatives for the error with respect to each weight, or other parameter in the model parameter data 1355, at each layer starting with the output layer. Chain rule can be utilized to iteratively compute the gradient with respect to each weight or parameter at each previous layer until all weight's gradients are computed. Next updated weights, or other parameters in the model parameter data 1355, are generated by updating each weight based on its corresponding calculated gradient. This process can be repeated on at least one input feature vector, which can include the same or different at least one feature vector used in the previous iteration, based on the updated weight vector and/or other updated parameters in the model parameter data 1355 to create a new updated weight vector and/or other new updated parameters in the model parameter data 1355. This process can continue to repeat until the output error converges, the output error is within a certain error threshold, or another criterion is reached to determine the most recently updated weight vector and/or other model parameter data 1355 is optimal or otherwise determined for selection.

Having determined the medical scan neural network and its final other model parameter data 1355, an inference step 1354 can be performed on new medical scans to produce inference data 1370, such as inferred output vectors, as shown in FIG. 7B. The inference step can include performing the forward propagation algorithm to propagate an input feature vector through a plurality of neural network layers based on the final model parameter data 1355, such as the weight values of the final weight vector, to produce the inference data. This inference step 1354 can correspond to performing the medical scan image analysis function, as defined by the final model parameter data 1355, on new medical scans to generate the inference data 1370, for example, in conjunction with the medical scan diagnosing system 108 to generate inferred diagnosis data or other selected output data for triaged medical scans based on its corresponding the input feature vector.

The inference step 1354 can include applying the density windowing step to new medical scans. Density window cut off values and/or a non-linear density windowing function that are learned can be automatically applied when performing the inference step. For example, if the training step 1352 was used to determine optimal upper density value cut off and/or lower density value cut off values to designate an optimal density window, the inference step 1354 can include masking pixels of incoming scans that fall outside of this determined density window before applying the forward propagation algorithm. As another example, if learned parameters of one or more convolutional functions correspond to the optimal upper density value cut off and/or lower density value cut off values, the density windowing step is inherently applied when the forward propagation algorithm is performed on the new medical scans.

In some embodiments where a medical scan analysis function is defined by model parameter data 1355 corresponding to a neutral network model, the neural network model can be a fully convolutional neural network. In such embodiments, only convolution functions are performed to propagate the input feature vector through the layers of the neural network in the forward propagation algorithm. This enables the medical scan image analysis functions to process input feature vectors of any size. For example, as discussed herein, the pixel data corresponding to the three-dimensional subregions is utilized input to the forward propagation algorithm when the training step 1352 is employed to populate the weight vector and/or other model parameter data 1355. However, when performing the forward propagation algorithm in the inference step 1354, the pixel data of full medical scans can be utilized as input, allowing the entire scan to be processed to detect and/or classify abnormalities, or otherwise generate the inference data 1370. This may be a preferred embodiment over other embodiments where new scans must also be sampled by selecting a three-dimensional subregions and/or other embodiments where the inference step requires “piecing together” inference data 1370 corresponding to multiple three-dimensional subregions processed separately.

The inferred output vector of the inference data 1370 can include a plurality of abnormality probabilities mapped to a pixel location of each of a plurality of cross-sectional image slices of the new medical scan. For example, the inferred output vector can indicate a set of probability matrices 1371, where each matrix in the set corresponds to one of the plurality of image slices of the medical scan, where each matrix is a size corresponding to the number of pixels in each image slice, where each cell of each matrix corresponds to a pixel of the corresponding image slice, whose value is the abnormality probability of the corresponding pixel.

A detection step 1372 can include determining if an abnormality is present in the medical scan based on the plurality of abnormality probabilities. Determining if an abnormality is present can include, for example, determining that a cluster of pixels in the same region of the medical scan correspond to high abnormality probabilities, for example, where a threshold proportion of abnormality probabilities must meet or exceed a threshold abnormality probability, where an average abnormality probability of pixels in the region must meet or exceed a threshold abnormality probability, where the region that includes the cluster of pixels must be at least a certain size, etc. Determining if an abnormality is present can also include calculating a confidence score based on the abnormality probabilities and/or other data corresponding to the medical scan such as patient history data. The location of the detected abnormality can be determined in the detection step 1372 based on the location of the pixels with the high abnormality probabilities. The detection step can further include determining an abnormality region 1373, such as a two-dimensional subregion on one or more image slices that includes some or all of the abnormality. The abnormality region 1373 determined in the detection step 1372 can be mapped to the medical scan to populate some or all of the abnormality location data 443 for use by one or more other subsystems 101 and/or client devices 120. Furthermore, determining whether or not an abnormality exists in the detection step 1372 can be used to populate some or all of the diagnosis data 440 of the medical scan, for example, to indicate that the scan is normal or contains an abnormality in the diagnosis data 440.

An abnormality classification step 1374 can be performed on a medical scan in response to determining an abnormality is present. Classification data 1375 corresponding to one or more classification categories such as abnormality size, volume, pre-post contract, doubling time, calcification, components, smoothness, texture, diagnosis data, one or more medical codes, a malignancy rating such as a Lung-RADS score, or other classifying data as described herein can be determined based on the detected abnormality. The classification data 1375 generated by the abnormality classification step 1374 can be mapped to the medical scan to populate some or all of the abnormality classification data 445 of the corresponding abnormality classifier categories 444 and/or abnormality pattern categories 446 and/or to determine one or more medical codes 447 of the medical scan. The abnormality classification step 1374 can include performing an abnormality classification function on the full medical scan, or the abnormality region 1373 determined in the detection step 1372. The abnormality classification function can be based on another model trained on abnormality data such as a support vector machine model, another neural network model, or any supervised classification model trained on medical scans, or portions of medical scans, that include known abnormality classifying data to generate inference data for some or all of the classification categories. For example, the abnormality classification function can include another medical scan analysis function. Classification data 1375 in each of a plurality of classification categories can also be assigned their own calculated confidence score, which can also be generated by utilizing the abnormality classification function. Output to the abnormality classification function can also include at least one identified similar medical scan and/or at least one identified similar cropped image, for example, based on the training data. The abnormality classification step can also be included in the inference step 1354, where the inferred output vector or other inference data 1370 of the medical scan image analysis function includes the classification data 1375.

The abnormality classification function can be trained on full medical scans and/or one or more cropped or full selected image slices from medical scans that contain an abnormality. For example, the abnormality classification function can be trained on a set of two-dimensional cropped slices that include abnormalities. The selected image slices and/or the cropped region in each selected image slice for each scan in the training set can be automatically selected based upon the known location of the abnormality. Input to the abnormality classification function can include the full medical scan, one or more selected full image slices, and/or one or more selected image slices cropped based on a selected region. Thus, the abnormality classification step can include automatically selecting one or more image slices that include the detected abnormality. The slice selection can include selecting the center slice in a set of consecutive slices that are determined to include the abnormality or selecting a slice that has the largest cross-section of the abnormality, or selecting one or more slices based on other criteria. The abnormality classification step can also include automatically generating one or more cropped two-dimensional images corresponding to the one or more of the selected image slices based on an automatically selected region that includes the abnormality.

Input to the abnormality classification function can also include other data associated with the medical scan, including patient history, risk factors, or other metadata. The abnormality classification step can also include determining some or all of the characteristics based on data of the medical scan itself. For example, the abnormality size and volume can be determined based on a number of pixels determined to be part of the detected abnormality. Other classifiers such as abnormality texture and/or smoothness can be determined by performing one or more other preprocessing functions on the image specifically designed to characterize such features. Such preprocessed characteristics can be included in the input to the abnormality classification function to the more difficult task of assigning a medical code or generating other diagnosis data. The training data can also be preprocessed to include such preprocessed features.

A similar scan identification step 1376 can also be performed on a medical scan with a detected abnormality and/or can be performed on the abnormality region 1373 determined in the detection step 1372. The similar scan identification step 1376 can include generating similar abnormality data 1377, for example, by identifying one or more similar medical scans or one or more similar cropped two-dimensional images from a database of medical scans and/or database of cropped two-dimensional images. Similar medical scans and/or cropped images can include medical scans or cropped images that are visually similar, medical scans or cropped images that have known abnormalities in a similar location to an inferred abnormality location of the given medical scan, medical scans that have known abnormalities with similar characteristics to inferred characteristics of an abnormality in the given scan, medical scans with similar patient history and/or similar risk factors, or some combination of these factors and/or other known and/or inferred factors. The similar abnormality data 1377 can be mapped to the medical scan to populate some or all of its corresponding similar scan data 480 for use by one or more other subsystems 101 and/or client devices 120.

The similar scans identification step 1376 can include performing a scan similarity algorithm, which can include generating a feature vector for the given medical scan and for medical scans in the set of medical scans, where the feature vector can be generated based on quantitative and/or category based visual features, inferred features, abnormality location and/or characteristics such as the predetermined size and/or volume, patient history and/or risk factor features, or other known or inferred features. A medical scan similarity analysis function can be applied to the feature vector of the given medical scan and one or more feature vectors of medical scans in the set. The medical scan similarity analysis function can include computing a similarity distance such as the Euclidian distance between the feature vectors, and assigning the similarity distance to the corresponding medical scan in the set. Similar medical scans can be identified based on determining one or more medical scans in the set with a smallest computed similarity distance, based on ranking medical scans in the set based on the computed similarity distances and identifying a designated number of top ranked medical scans, and/or based on determining if a similarity distance between the given medical scan and a medical scan in the set is smaller than a similarity threshold. Similar medical scans can also be identified based on determining medical scans in a database that mapped to a medical code that matches the medical code of the medical scan, or mapped to other matching classifying data. A set of identified similar medical scans can also be filtered based on other inputted or automatically generated criteria, where for example only medical scans with reliable diagnosis data or rich patient reports, medical scans with corresponding with longitudinal data in the patient file such as multiple subsequent scans taken at later dates, medical scans with patient data that corresponds to risk factors of the given patient, or other identified criteria, where only a subset of scans that compare favorably to the criteria are selected from the set and/or only a highest ranked single scan or subset of scans are selected from the set, where the ranking is automatically computed based on the criteria. Filtering the similar scans in this fashion can include calculating, or can be based on previously calculated, one or more scores as discussed herein. For example, the ranking can be based on a longitudinal quality score, such as the longitudinal quality score 434, which can be calculated for an identified medical scan based on a number of subsequent and/or previous scans for the patient. Alternatively or in addition, the ranking can be based on a confidence score associated with diagnosis data of the scan, such as confidence score data 460, based on performance score data associated with a user or medical entity associated with the scan, based on an amount of patient history data or data in the medical scan entry 352, or other quality factors. The identified similar medical scans can be filtered based on ranking the scans based on their quality score and/or based on comparing their quality score to a quality score threshold. In some embodiments, a longitudinal threshold must be reached, and only scans that compare favorably to the longitudinal threshold will be selected. For example, only scans with at least three scans on file for the patient and final biopsy data will be included.

In some embodiments, the similarity algorithm can be utilized in addition to or instead of the trained abnormality classification function to determine some or all of the inferred classification data 1375 of the medical scan, based on the classification data such as abnormality classification data 445 or other diagnosis data 440 mapped to one or more of the identified similar scans. In other embodiments, the similarity algorithm is merely used to identify similar scans for review by medical professionals to aid in review, diagnosis, and/or generating medical reports for the medical image.

A display parameter step 1378 can be performed based on the detection and/or classification of the abnormality. The display parameter step can include generating display parameter data 1379, which can include parameters that can be used by an interactive interface to best display each abnormality. The same or different display parameters can be generated for each abnormality. The display parameter data generated in the display parameter step 1378 can be mapped to the medical scan to populate some or all of its corresponding display parameter data 470 for use by one or more other subsystems 101 and/or client devices 120.

Performing the display parameter step 1378 can include selecting one or more image slices that include the abnormality by determining the one or more image slices that include the abnormality and/or determining one or more image slices that has a most optimal two-dimensional view of the abnormality, for example by selecting the center slice in a set of consecutive slices that are determined to include the abnormality, selecting a slice that has the largest cross-section of the abnormality, selecting a slice that includes a two-dimensional image of the abnormality that is most similar to a selected most similar two-dimensional-image, selecting the slice that was used as input to the abnormality classification step and/or similar scan identification step, or based on other criteria. This can also include automatically cropping one or more selected image slices based on an identified region that includes the abnormality. This can also select an ideal Hounsfield window that best displays the abnormality. This can also include selecting other display parameters based on data generated by the medical scan interface evaluating system and based on the medical scan.

FIGS. 8A-8F illustrate embodiments of a medical picture archive integration system 2600. The medical picture archive integration system 2600 can provide integration support for a medical picture archive system 2620, such as a PACS that stores medical scans. The medical picture archive integration system 2600 can utilize model parameters received from a central server system 2640 via a network 2630 to perform an inference function on de-identified medical scans of medical scans received from the medical picture archive system 2620. The annotation data produced by performing the inference function can be transmitted back to the medical picture archive system. Furthermore, the annotation data and/or de-identified medical scans can be sent to the central server system 2640, and the central server system can train on this information to produce new and/or updated model parameters for transmission back to the medical picture archive integration system 2600 for use on subsequently received medical scans.

In various embodiments, medical picture archive integration system 2600 includes a de-identification system that includes a first memory designated for protected health information (PHI), operable to perform a de-identification function on a DICOM image, received from a medical picture archive system, to identify at least one patient identifier and generate a de-identified medical scan that does not include the at least one patient identifier. The medical picture archive integration system further includes a de-identified image storage system that stores the de-identified medical scan in a second memory that is separate from the first memory, and an annotating system, operable to utilize model parameters received from a central server to perform an inference function on the de-identified medical scan, retrieved from the second memory to generate annotation data for transmission to the medical picture archive system as an annotated DICOM file.

The first memory and the second memory can be implemented by utilizing separate storage systems: the first memory can be implemented by a first storage system designated for PHI storage, and the second memory can be implemented by a second storage system designated for storage of de-identified data. The first storage system can be protected from access by the annotating system, while the second storage system can be accessible by the annotating system. The medical picture archive integration system 2600 can be operable to perform the de-identification function on data in first storage system to generate de-identified data. The de-identified data can then be stored in the second storage system for access by the annotating system. The first and second storage systems can be physically separate, each utilizing at least one of their own, separate memory devices. Alternatively, the first and second storage systems can be virtually separate, where data is stored in separate virtual memory locations on the same set of memory devices. Firewalls, virtual machines, and/or other protected containerization can be utilized to enforce the separation of data in each storage system, to protect the first storage system from access by the annotating system and/or from other unauthorized access, and/or to ensure that only data of the first storage system that has been properly de-identified through application of the de-identification function can be stored in the second storage system.

As shown in FIG. 8A, the medical picture archive system 2620 can receive image data from a plurality of modality machines 2622, such as CT machines, MRI machines, x-ray machines, and/or other medical imaging machines that produce medical scans. The medical picture archive system 2620 can store this image data in a DICOM image format and/or can store the image data in a plurality of medical scan entries 352 as described in conjunction with some or all of the attributes described in conjunction with FIGS. 4A and 4B. While “DICOM image” will be used herein to refer to medical scans stored by the medical picture archive system 2620, the medical picture archive integration system 2600 can provide integration support for medical picture archive systems 2620 that store medical scans in other formats.

The medical picture archive integration system 2600 can include a receiver 2602 and a transmitter 2604, operable to transmit and receive data from the medical picture archive system 2620, respectively. For example, the receiver 2602 and transmitter 2604 can be configured to receive and transmit data, respectively, in accordance with a DICOM communication protocol and/or another communication protocol recognized by the medical picture archive system 2620. The receiver can receive DICOM images from the medical picture archive system 2620. The transmitter 2604 can send annotated DICOM files to the medical picture archive system 2620.

DICOM images received via receiver 2602 can be sent directly to a de-identification system 2608. The de-identification system 2608 can be operable to perform a de-identification function on the first DICOM image to identify at least one patient identifier in the DICOM image, and to generate a de-identified medical scan that does not include the identified at least one patient identifier. As used herein, a patient identifier can include any patient identifying data in the image data, header, and/or metadata of a medical scan, such as a patient ID number or other unique patient identifier, an accession number, a service-object pair (SOP) instance unique identifier (UID) field, scan date and/or time that can be used to determine the identity of the patient that was scanned at that date and/or time, and/or other private data corresponding to the patient, doctor, or hospital. In some embodiments, the de-identified medical scan is still in a DICOM image format. For example, a duplicate DICOM image that does not include the patient identifiers can be generated, and/or the original DICOM image can be altered such that the patient identifiers of the new DICOM image are masked, obfuscated, removed, replaced with a custom fiducial, and/or otherwise anonymized. In other embodiments, the de-identified medical scan is formatted in accordance with a different image format and/or different data format that does not include the identifying information. In some embodiments, other private information, for example, associated with a particular doctor or other medical professional, can be identified and anonymized as well.

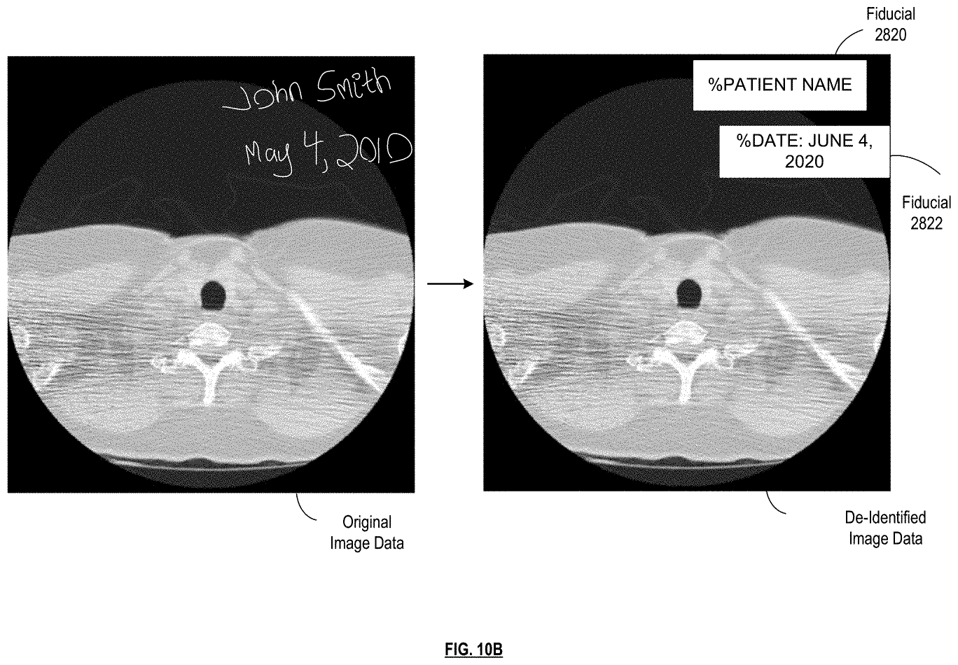

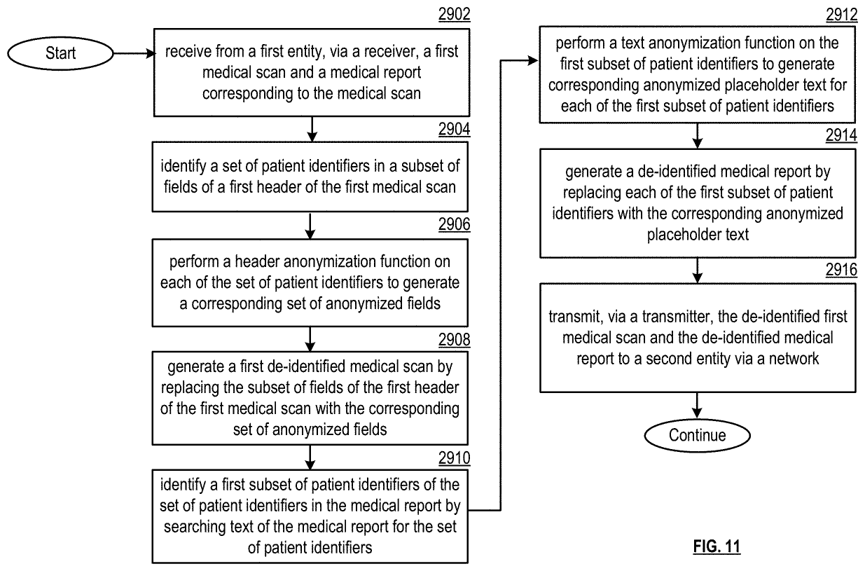

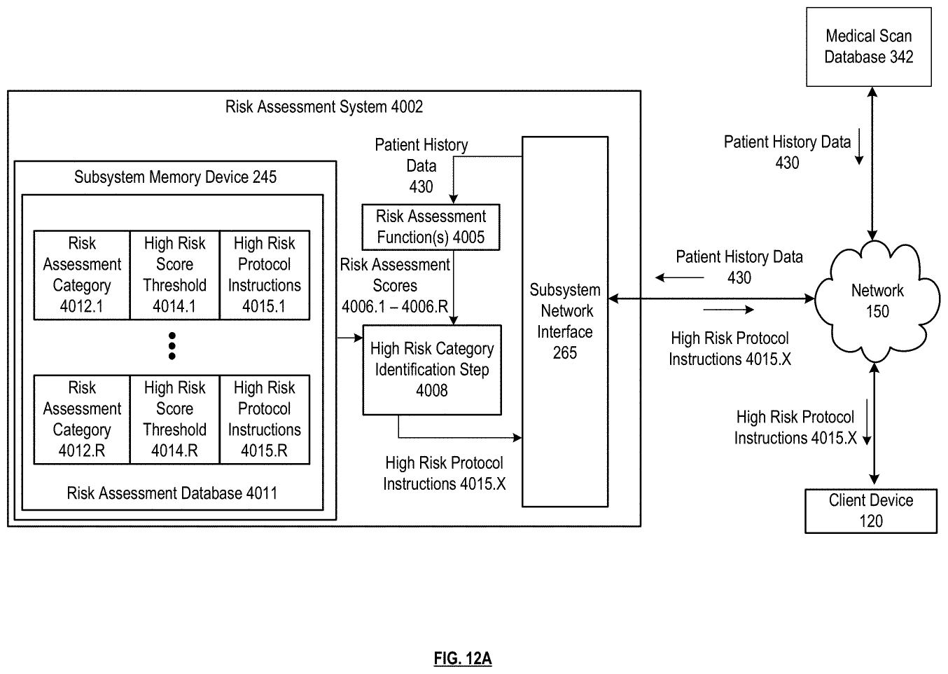

Some patient identifying information can be included in a DICOM header of the DICOM image, for example, in designated fields for patient identifiers. These corresponding fields can be anonymized within the corresponding DICOM header field. Other patient identifying information can be included in the image itself, such as in medical scan image data 410. For example, the image data can include a patient name or other identifier that was handwritten on a hard copy of the image before the image was digitized. As another example, a hospital administered armband or other visual patient information in the vicinity of the patient may have been captured in the image itself. A computer vision model can detect the presence of these identifiers for anonymization, for example, where a new DICOM image includes a fiducial image that covers the identifying portion of the original DICOM image. In some embodiments, patient information identified in the DICOM header can be utilized to detect corresponding patient information in the image itself. For example, a patient name extracted from the DICOM header before anonymization can be used to search for the patient name in the image and/or to detect a location of the image that includes the patient name. In some embodiments, the de-identification system 2608 is implemented by the de-identification system discussed in conjunction with FIGS. 10A, 10B and 11, and/or utilizes functions and/or operations discussed in conjunction with FIGS. 10A, 10B and 11.

The de-identified medical scan can be stored in de-identified image storage system 2610 and the annotating system 2612 can access the de-identified medical scan from the de-identified image storage system 2610 for processing. The de-identified storage system can archive a plurality of de-identified DICOM images and/or can serve as temporary storage for the de-identified medical scan until processing of the de-identified medical scan by the annotating system 2612 is complete. The annotating system 2612 can generate annotation data by performing an inference function on the de-identified medical scan, utilizing the model parameters received from the central server system 2640. The annotation data can correspond to some or all of the diagnosis data 440 as discussed in conjunction with FIGS. 4A and 4B. In come embodiments, the annotating system 2612 can utilize the model parameters to perform inference step 1354, the detection step 1372, the abnormality classification step 1374, the similar scan identification step 1376, and/or the display parameter step 1378 of the medical scan image analysis system 112, as discussed in conjunction with FIG. 7B, on de-identified medical scans received from the medical picture archive system 2620.

In some embodiments, model parameters for a plurality of inference functions can be received from the central server system 2640, for example, where each inference function corresponds to one of a set of different scan categories. Each scan category can correspond to a unique combination of one or a plurality of scan modalities, one of a plurality of anatomical features, and/or other scan classifier data 420. For example, a first inference function can be trained on and intended for de-identified medical scans corresponding chest CT scans, and a second inference function can be trained on and intended for de-identified medical scans corresponding to head MRI scans. The annotating system can select one of the set of inference functions based on determining the scan category of the DICOM image, indicated in the de-identified medical scan, and selecting the inference function that corresponds to the determined scan category.

To ensure that scans received from the medical picture archive system 2620 match the set of scan categories for which the annotating system is operable to perform a corresponding inference function, the transmitter can transmit requests, such as DICOM queries, indicating image type parameters such as parameters corresponding to scan classifier data 420, for example indicating one or more scan modalities, one or more anatomical regions, and/or other parameters. For example, the request can indicate that all incoming scans that match the set of scan categories corresponding to a set of inference functions the annotating system 2612 for which the annotating system has obtained model parameters from the central server system 2640 and is operable to perform.

Once the annotation data is generated by performing the selected inference function, the annotating system 2612 can generate an annotated DICOM file for transmission to the medical picture archive system 2620 for storage. The annotated DICOM file can include some or all of the fields of the diagnosis data 440 and/or abnormality annotation data 442 of FIGS. 4A and 4B. The annotated DICOM file can include scan overlay data, providing location data of an identified abnormality and/or display data that can be used in conjunction with the original DICOM image to indicate the abnormality visually in the DICOM image and/or to otherwise visually present the annotation data, for example, for use with the medical scan assisted review system 102. For example, a DICOM presentation state file can be generated to indicate the location of an abnormality identified in the de-identified medical scan. The DICOM presentation state file can include an identifier of the original DICOM image, for example, in metadata of the DICOM presentation state file, to link the annotation data to the original DICOM image. In other embodiments, a full, duplicate DICOM image is generated that includes the annotation data with an identifier linking this duplicate annotated DICOM image to the original DICOM image.

The identifier linking the annotated DICOM file to the original DICOM image can be extracted from the original DICOM file by the de-identification system 2608, thus enabling the medical picture archive system 2620 to link the annotated DICOM file to the original DICOM image in its storage. For example, the de-identified medical scan can include an identifier that links the de-identified medical scan to the original DICOM file, but does not link the de-identified medical scan to a patient identifier or other private data.

In some embodiments, generating the annotated DICOM file includes altering one or more fields of the original DICOM header. For example, standardized header formatting function parameters can be received from the central server system and can be utilized by the annotating system to alter the original DICOM header to match a standardized DICOM header format. The standardized header formatting function can be trained in a similar fashion to other medical scan analysis functions discussed herein and/or can be characterized by some or all fields of a medical scan analysis function entry 356. The annotating system can perform the standardized header formatting function on a de-identified medical scan to generate a new, standardized DICOM header for the medical scan to be sent back to the medical picture archive system 2620 in the annotated DICOM file and/or to replace the header of the original DICOM file. The standardized header formatting function can be run in addition to other inference functions utilized to generate annotation data. In other embodiments, the medical picture archive integration system 2600 is implemented primarily for header standardization for medical scans stored by the medical picture archive system 2620. In such embodiments, only the standardized header formatting function is performed on the de-identified data to generate a modified DICOM header for the original DICOM image, but the de-identified medical scan is not annotated.

In some embodiments of header standardization, the annotation system can store a set of acceptable, standardized entries for some or all of the DICOM header fields, and can select one of the set of acceptable, standardized entries in populating one or more fields of the new DICOM header for the annotated DICOM file. For example, each of the set of scan categories determined by the annotating system can correspond to a standardized entry of one or more fields of the DICOM header. The new DICOM header can thus be populated based on the determined scan category.

In some embodiments, each of the set of standardized entries can be mapped to a set of related, non-standardized entries, such as entries in a different order, commonly misspelled entries, or other similar entries that do not follow a standardized format. For example, one of the set of acceptable, standardized entries for a field corresponding to a scan category can include “Chest CT”, which can be mapped to a set of similar, non-standardized entries which can include “CT chest”, “computerized topography CT”, and/or other entries that are not standardized. In such embodiments, the annotating system can determine the original DICOM header is one of the similar non-standardized entries, and can select the mapped, standardized entry as the entry for the modified DICOM header. In other embodiments, the image data itself and/or or other header data can be utilized by the annotation system to determine a standardized field. For example, an input quality assurance function 1106 can be trained by the central server system and sent to the annotating system to determine one or more appropriate scan classifier fields, or one or more other DICOM header fields, based on the image data or other data of the de-identified medical scan. One or more standardized labels can be assigned to corresponding fields of the modified DICOM header based on the one or more fields determined by the input quality assurance function.

In some embodiments, the DICOM header is modified based on the annotation data generated in performing the inference function. In particular, a DICOM priority header field can be generated and/or modified automatically based on the severity and/or time-sensitivity of the abnormalities detected in performing the inference function. For example, a DICOM priority header field can be changed from a low priority to a high priority in response to annotation data indicating a brain bleed in the de-identified medical scan of a DICOM image corresponding to a head CT scan, and a new DICOM header that includes the high priority DICOM priority header field can be sent back to the medical picture archive system 2620 to replace or otherwise be mapped to the original DICOM image of the head CT scan.

In various embodiments, the medical picture archive system 2620 is disconnected from network 2630, for example, to comply with requirements regarding Protected Health Information (PHI), such as patient identifiers and other private patient information included in the DICOM images and/or otherwise stored by the medical picture archive system 2620. The medical picture archive integration system 2600 can enable processing of DICOM images while still protecting private patient information by first de-identifying DICOM data by utilizing de-identification system 2608. The de-identification system 2608 can utilize designated processors and memory of the medical picture archive integration system, for example, designated for PHI. The de-identification system 2608 can be decoupled from the network 2630 to prevent the DICOM images that still include patient identifiers from being accessed via the network 2630. For example, as shown in FIG. 8A, the de-identification system 2608 is not connected to network interface 2606. Furthermore, only the de-identification system 2608 has access to the original DICOM files received from the medical picture archive system 2620 via receiver 2602. The de-identified image storage system 2610 and annotating system 2612, as they are connected to network 2630 via network interface 2606, only store and have access to the de-identified medical scan produced by the de-identification system 2608.

This containerization that separates the de-identification system 2608 from the de-identified image storage system 2610 and the annotating system 2612 is further illustrated in FIG. 8B, which presents an embodiment of the medical picture archive integration system 2600. The de-identification system 2608 can include its own designated memory 2654 and processing system 2652, connected to receiver 2602 via bus 2659. For example, this memory 2654 and processing system 2652 can be designated for PHI, and can adhere to requirements for handling PHI. The memory 2654 can store executable instructions that, when executed by the processing system 2652, enable the de-identification system to perform the de-identification function on DICOM images received via receiver 2602 of the de-identification system. The incoming DICOM images can be temporarily stored in memory 2654 for processing, and patient identifiers detected in performing the de-identification function can be temporarily stored in memory 2654 to undergo anonymization. Interface 2655 can transmit the de-identified medical scan to interface 2661 for use by the de-identified image storage system 2610 and the annotating system 2612. Interface 2655 can be protected from transmitting original DICOM files and can be designated for transmission of de-identified medical scan only.

Bus 2669 connects interface 2661, as well as transmitter 2604 and network interface 2606, to the de-identified image storage system 2610 and the annotating system 2612. The de-identified image storage system 2610 and annotating system 2612 can utilize separate processors and memory, or can utilize shared processors and/or memory. For example, the de-identified image storage system 2610 can serve as temporary memory of the annotating system 2612 as de-identified images are received and processed to generate annotation data.

As depicted in FIG. 8B, the de-identified image storage system 2610 can include memory 2674 that can temporarily store incoming de-identified medical scans as it undergoes processing by the annotating system 2612 and/or can archive a plurality of de-identified medical scans corresponding to a plurality of DICOM images received by the medical picture archive integration system 2600. The annotating system 2612 can include a memory 2684 that stores executable instructions that, when executed by processing system 2682, cause the annotating system 2612 perform a first inference function on de-identified medical scan to generate annotation data by utilizing the model parameters received via interface 2606, and to generate an annotated DICOM file based on the annotation data for transmission via transmitter 2604. The model parameters can be stored in memory 2684, and can include model parameters for a plurality of inference functions, for example, corresponding to a set of different scan categories.

The medical picture archive integration system can be an onsite system, installed at a first geographic site, such as a hospital or other medical entity that is affiliated with the medical picture archive system 2620. The hospital or other medical entity can further be responsible for the PHI of the de-identification system, for example, where the memory 2654 and processing system 2652 are owned by, maintained by, and/or otherwise affiliated with the hospital or other medical entity. The central server system 2640 can be located at a second, separate geographic site that is not affiliated with the hospital or other medical entity and/or at a separate geographic site that is not affiliated with the medical picture archive system 2620. The central server system 2640 can be a server configured to be outside the network firewall and/or out outside the physical security of the hospital or other medical entity or otherwise not covered by the particular administrative, physical and technical safeguards of the hospital or other medical entity.

FIG. 8C further illustrates how model parameters can be updated over time to improve existing inference functions and/or to add new inference functions, for example corresponding to new scan categories. In particular, the some or all of the de-identified medical scans generated by the de-identification system 2608 can be transmitted back to the central server system, and the central server system 2640 can train on this data to improve existing models by producing updated model parameters of an existing inference function and/or to generate new models, for example, corresponding to new scan categories, by producing new model parameters for new inference functions. For example, the central server system 2640 can produce updated and/or new model parameters by performing the training step 1352 of the medical scan image analysis system 112, as discussed in conjunction with FIG. 7A, on a plurality of de-identified medical scans received from the medical picture archive integration system 2600.

The image type parameters can be determined by the central server system to dictate characteristics of the set of de-identified medical scans to be received to train and/or retrain the model. For example, the image type parameters can correspond to one or more scan categories, can indicate scan classifier data 420, and/or can indicate one or more scan modalities, one or more anatomical regions, a date range, and/or other parameters. The image type parameters can be determined by the central server system based on training parameters 620 determined for the corresponding inference function to be trained, and/or based on characteristics of a new and/or existing scan category corresponding to the inference function to be trained. The image type parameters can be sent to the medical picture archive integration system 2600, and a request such as a DICOM query can be sent to the medical picture archive system 2620, via transmitter 2604, that indicates the image type parameters. For example, the processing system 2682 can be utilized to generate the DICOM query based on the image type parameters received from the central server system 2640. The medical picture archive system can automatically transmit one or more DICOM images to the medical picture archive integration system in response to determining that the one or more DICOM images compares favorably to the image type parameters. The DICOM images received in response can be de-identified by the de-identification system 2608. In some embodiments, the de-identified medical scans can be transmitted directly to the central server system 2640, for example, without generating annotation data.

The central server system can generate the new and/or updated model parameters by training on the received set of de-identified medical scans, and can transmit the new and/or updated model parameters to the de-identified storage system. If the model parameters correspond to a new inference function for a new scan category, the medical picture archive integration system 2600 can generate a request, such as a DICOM query, for transmission to the medical picture archive system indicating that incoming scans corresponding to image type parameters corresponding to the new scan category be sent to the medical picture archive integration system. The annotating system can update the set of inference functions to include the new inference function, and the annotating system can select the new inference function from the set of inference functions for subsequently generated de-identified medical scans by the de-identification system by determining each of these de-identified medical scans indicate the corresponding DICOM image corresponds to the new scan category. The new model parameters can be utilized to perform the new inference function on each of these de-identified medical scans to generate corresponding annotation data, and an annotated DICOM file corresponding to each of these de-identified medical scans can be generated for transmission to the medical picture archive system via the transmitter.

In some embodiments, the central server system 2640 receives a plurality of de-identified medical scans from a plurality of medical picture archive integration system 2600, for example, each installed at a plurality of different hospitals or other medical entities, via the network 2630. The central server system can generate training sets by integrating de-identified medical scans from some or all of the plurality of medical picture archive integration systems 2600 to train one or more inference functions and generate model parameters. The plurality of medical picture archive integration systems 2600 can utilize the same set of inference functions or different sets of inference functions. In some embodiments, the set of inference functions utilized by the each of the plurality of medical picture archive systems 2620 are trained on different sets of training data. For example, the different sets of training data can correspond to the set of de-identified medical scans received from the corresponding medical picture archive integration system 2600.

In some embodiments, the medical scan diagnosing system 108 can be utilized to implement the annotating system 2612, where the corresponding subsystem processing device 235 and subsystem memory device 245 of the medical scan diagnosing system 108 are utilized to implement the processing system 2682 and the memory 2684, respectively. Rather than receiving the medical scans via the network 150 as discussed in conjunction with FIG. 6A, the medical scan diagnosing system 108 can perform a selected medical scan inference function 1105 on an incoming de-identified medical scan generated by the de-identification system 2608 and/or retrieved from the de-identified image storage system 2610. Memory 2684 can store the set of medical scan inference functions 1105, each corresponding to a scan category 1120, where the inference function is selected from the set based on determining the scan category of the de-identified medical scan and selecting the corresponding inference function. The processing system 2682 can perform the selected inference function 1105 to generate the inference data 1110, which can be further utilized by the annotating system 2612 to generate the annotated DICOM file for transmission back to the medical picture archive system 2620. New medical scan inference functions 1105 can be added to the set when corresponding model parameters are received from the central server system. The remediation step 1140 can be performed locally by the annotating system 2612 and/or can be performed by the central server system 2640 by utilizing one or more de-identified medical scans and corresponding annotation data sent to the central server system 2640. Updated model parameters can be generated by the central server system 2640 and sent to the medical picture archive integration system 2600 as a result of performing the remediation step 1140.

The central server system 2640 can be implemented by utilizing one or more of the medical scan subsystems 101, such as the medical scan image analysis system 112 and/or the medical scan diagnosing system 108, to produce model parameters for one or more inference functions. The central server system can store or otherwise communicate with a medical scan database 342 that includes the de-identified medical scans and/or annotation data received from one or more medical picture archive integration systems 2600. Some or all entries of the medical scan database 342 can be utilized to as training data to produce model parameters for one or more inference functions. These entries of the medical scan database 342 can be utilized by other subsystems 101 as discussed herein. For example, other subsystems 101 can utilize the central server system 2640 to fetch medical scans and/or corresponding annotation data that meet specified criteria. The central server system 2640 can query the medical picture archive integration system 2600 based on this criteria, and can receive de-identified medical scans and/or annotation data in response. This can be sent to the requesting subsystem 101 directly and/or can be added to the medical scan database 342 or another database of the database storage system 140 for access by the requesting subsystem 101.

Alternatively or in addition, the central server system 2640 can store or otherwise communicate with a user database 344 storing user profile entries corresponding to each of a plurality of medical entities that each utilize a corresponding one of a plurality of medical picture archive integration systems 2600. For example, basic user data corresponding to the medical entity can be stored as basic user data, a number of scans or other consumption information indicating usage of one or more inference functions by corresponding medical picture archive integration system can be stored as consumption usage data, and/or a number of scans or other contribution information indicating de-identified scans sent to the central server system as training data can be stored as contribution usage data. The user profile entry can also include inference function data, for example, with a list of model parameters or function identifiers, such as medical scan analysis function identifiers 357, of inference functions currently utilized by the corresponding medical picture archive integration system 2600. These entries of the user database 344 can be utilized by other subsystems 101 as discussed herein.

Alternatively or in addition, the central server system 2640 can store or otherwise communicate with a medical scan analysis function database 346 to store model parameters, training data, or other information for one or more inference functions as medical scan analysis function entries 356. In some embodiments, model parameter data 623 can indicate the model parameters and function classifier data 610 can indicate the scan category of inference function entries. In some embodiments, the medical scan analysis function entry 356 can further include usage identifying information indicating a medical picture archive integration system identifier, medical entity identifier, and/or otherwise indicating which medical archive integration systems and/or medical entities have received the corresponding model parameters to utilize the inference function corresponding to the medical scan analysis function entry 356. These entries of the medical scan analysis function database 346 can be utilized by other subsystems 101 as discussed herein.

In some embodiments, the de-identification function is a medical scan analysis function, for example, with a corresponding medical scan analysis function entry 356 in the medical scan analysis function database 346. In some embodiments, the de-identification function is trained by the central server system 2640. For example, the central server system 2640 can send de-identification function parameters to the medical picture archive integration system 2600 for use by the de-identification system 2608. In embodiments with a plurality of medical picture archive integration systems 2600, each of the plurality of medical picture archive integration systems 2600 can utilize the same or different de-identification functions. In some embodiments, the de-identification function utilized by the each of the plurality of medical picture archive integration systems 2600 are trained on different sets of training data. For example, the different sets of training data can correspond to each different set of de-identified medical scans received from each corresponding medical picture archive integration system 2600.

In some embodiments, as illustrated in FIGS. 8D-8F, the medical picture archive integration system 2600 can further communicate with a report database 2625, such as a Radiology Information System (RIS), that includes a plurality of medical reports corresponding to the DICOM images stored by the medical picture archive system 2620.

As shown in FIG. 8D, the medical picture archive integration system 2600 can further include a receiver 2603 that receives report data, corresponding to the DICOM image, from report database 2625. The report database 2625 can be affiliated with the medical picture archive system 2620 and can store report data corresponding to DICOM images stored in the medical picture archive system. The report data of report database 2625 can include PHI, and the report database 2625 can thus be disconnected from network 2630.

The report data can include natural language text, for example, generated by a radiologist that reviewed the corresponding DICOM image. The report data can be used to generate the de-identified medical scan, for example, where the de-identification system 2608 performs a natural language analysis function on the report data to identify patient identifying text in the report data. The de-identification system 2608 can utilize this patient identifying text to detect matching patient identifiers in the DICOM image to identify the patient identifiers of the DICOM image and generate the de-identified medical scan. In some embodiments, the report data can be de-identified by obfuscating, hashing, removing, replacing with a fiducial, or otherwise anonymizing the identified patient identifying text to generate de-identified report data.

The de-identified report data can be utilized by the annotating system 2612, for example, in conjunction with the DICOM image, to generate the annotation data. For example, the annotating system 2612 can perform a natural language analysis function on the de-identified natural language text of the report data to generate some or all of the annotation data. In some embodiments, the de-identified report data is sent to the central server system, for example, to be used as training data for inference functions, for natural language analysis functions, for other medical scan analysis functions, and/or for use by at least one other subsystem 101. For example, other subsystems 101 can utilize the central server system 2640 to fetch medical reports that correspond to particular medical scans or otherwise meet specified criteria. The central server system 2640 can query the medical picture archive integration system 2600 based on this criteria, and can receive de-identified medical reports in response. This can be sent to the requesting subsystem 101 directly, can be added to the medical scan database 342, a de-identified report database, or another database of the database storage system 140 for access by the requesting subsystem 101.

In some embodiments the medical picture archive integration system 2600 can query the report database 2625 for the report data corresponding to a received DICOM image by utilizing a common identifier extracted from the DICOM image.

In some embodiments, the report data can correspond to a plurality of DICOM images. For example, the report data can include natural language text describing a plurality of medical scans of a patient that can include multiple sequences, multiple modalities, and/or multiple medical scans taken over time. In such embodiments, the patient identifying text and/or annotation data detected in the report data can also be applied to de-identify and/or generate annotation data for the plurality of DICOM images it describes. In such embodiments, the medical picture archive integration system 2600 can query the medical picture archive system 2620 for one or more additional DICOM images corresponding to the report data, and de-identified data and annotation data for these additional DICOM images can be generated accordingly by utilizing the report data.

In some embodiments, as shown in FIG. 8E, the medical picture archive system 2620 communicates with the report database 2625. The medical picture archive system 2620 can request the report data corresponding to the DICOM image from the report database 2625, and can transmit the report data to the medical picture archive integration system 2600 via a DICOM communication protocol for receipt via receiver 2602. The medical picture archive system 2620 can query the report database 2625 for the report data, utilizing a common identifier extracted from the corresponding DICOM image, in response to determining to send the corresponding DICOM image to the medical picture archive integration system 2600.

FIG. 8F presents an embodiment where report data is generated by the annotating system 2612 and is transmitted, via a transmitter 2605, to the report database 2625, for example via a DICOM communication protocol or other protocol recognized by the report database 2625. In other embodiments, the report data is instead transmitted via transmitter 2604 to the medical picture archive system 2620, and the medical picture archive system 2620 transmits the report data to the report database 2625.

The report data can be generated by the annotating system 2612 as output of performing the inference function on the de-identified medical scan. The report data can include natural language text data 448 generated automatically based on other diagnosis data 440 such as abnormality annotation data 442 determined by performing the inference function, for example, by utilizing a medical scan natural language generating function trained by the medical scan natural language analysis system 114. The report data can be generated instead of, or in addition to, the annotated DICOM file.

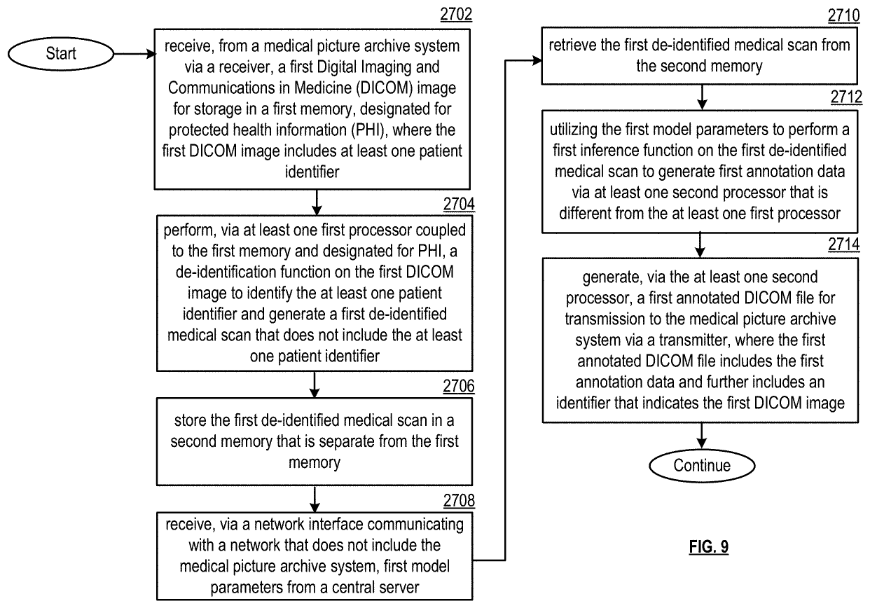

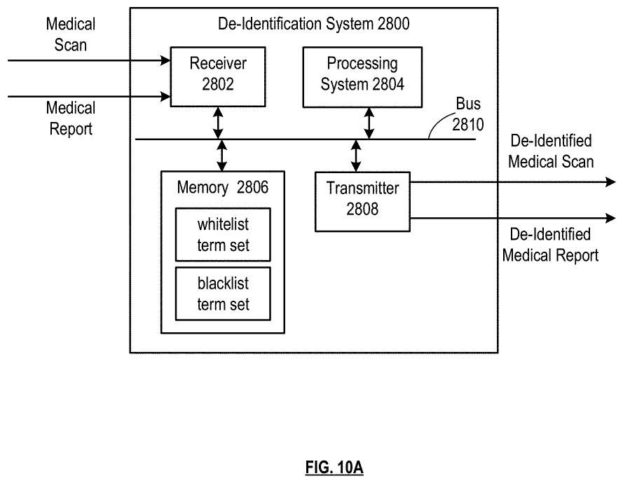

FIG. 9 presents a flowchart illustrating a method for execution by a medical picture archive integration system 2600 that includes a first memory and a second memory that store executional instructions that, when executed by at least one first processor and at least one second processor, respectfully, cause the medical picture archive integration system to perform the steps below. In various embodiments, the first memory and at least one first processor are implemented by utilizing, respectfully, the memory 2654 and processing system 2652 of FIG. 8B. In various embodiments, the second memory is implemented by utilizing the memory 2674 and/or the memory 2684 of FIG. 8B. In various embodiments, the at least one second processor is implemented by utilizing the processing system 2682 of FIG. 8B.

Step 2702 includes receiving, from a medical picture archive system via a receiver, a first DICOM image for storage in the first memory, designated for PHI, where the first DICOM image includes at least one patient identifier. Step 2704 includes performing, via at least one first processor coupled to the first memory and designated for PHI, a de-identification function on the first DICOM image to identify the at least one patient identifier and generate a first de-identified medical scan that does not include the at least one patient identifier.

Step 2706 includes storing the first de-identified medical scan in a second memory that is separate from the first memory. Step 2708 includes receiving, via a network interface communicating with a network that does not include the medical picture archive system, first model parameters from a central server.

Step 2710 includes retrieving the first de-identified medical scan from the second memory. Step 2712 includes utilizing the first model parameters to perform a first inference function on the first de-identified medical scan to generate first annotation data via at least one second processor that is different from the at least one first processor. Step 2714 includes generating, via the at least one second processor, a first annotated DICOM file for transmission to the medical picture archive system via a transmitter, where the first annotated DICOM file includes the first annotation data and further includes an identifier that indicates the first DICOM image. In various embodiments, the first annotated DICOM file is a DICOM presentation state file.

In various embodiments, the second memory further includes operational instructions that, when executed by the at least one second processor, further cause the medical picture archive integration system to retrieve a second de-identified medical scan from the de-identified image storage system, where the second de-identified medical scan was generated by the at least one first processor by performing the de-identification function on a second DICOM image received from the medical picture archive system. The updated model parameters are utilized to perform the first inference function on the second de-identified medical scan to generate second annotation data. A second annotated DICOM file is generated for transmission to the medical picture archive system via the transmitter, where the second annotated DICOM file includes the second annotation data and further includes an identifier that indicates the second DICOM image.

In various embodiments, the second memory stores a plurality of de-identified medical scans generated by the at least one first processor by performing the de-identification function on a corresponding plurality of DICOM images received from the medical picture archive system via the receiver. The plurality of de-identified medical scans is transmitted to the central server via the network interface, and the central server generates the first model parameters by performing a training function on training data that includes the plurality of de-identified medical scans.

In various embodiments, the central server generates the first model parameters by performing a training function on training data that includes a plurality of de-identified medical scans received from a plurality of medical picture archive integration systems via the network. Each of the plurality of medical picture archive integration systems communicates bidirectionally with a corresponding one of a plurality of medical picture archive systems, and the plurality of de-identified medical scans corresponds to a plurality of DICOM images stored by the plurality of medical picture archive integration systems.

In various embodiments, the first de-identified medical scan indicates a scan category of the first DICOM image. The second memory further stores operational instructions that, when executed by the at least one second processor, further cause the medical picture archive integration system to select the first inference function from a set of inference functions based on the scan category. The set of inference functions corresponds to a set of unique scan categories that includes the scan category. In various embodiments, each unique scan category of the set of unique scan categories is characterized by one of a plurality of modalities and one of a plurality of anatomical features.

In various embodiments, the first memory further stores operational instructions that, when executed by the at least one first processor, further cause the medical picture archive integration system to receive a plurality of DICOM image data from the medical picture archive system via the receiver for storage in the first memory in response to a query transmitted to the medical picture archive system via the transmitter. The query is generated by the medical picture archive integration system in response to a request indicating a new scan category received from the central server via the network. The new scan category is not included in the set of unique scan categories, and the plurality of DICOM image data corresponds to the new scan category. The de-identification function is performed on the plurality of DICOM image data to generate a plurality of de-identified medical scans for transmission to the central server via the network.

The second memory further stores operational instructions that, when executed by the at least one second processor, further cause the medical picture archive integration system to receive second model parameters from the central server via the network for a new inference function corresponding to the new scan category. The set of inference functions is updated to include the new inference function. The second de-identified medical scan is retrieved from the first memory, where the second de-identified medical scan was generated by the at least one first processor by performing the de-identification function on a second DICOM image received from the medical picture archive system. The new inference function is selected from the set of inference functions by determining the second de-identified medical scan indicates the second DICOM image corresponds to the new scan category. The second model parameters are utilized to perform the new inference function on the second de-identified medical scan to generate second annotation data. A second annotated DICOM file is generated for transmission to the medical picture archive system via the transmitter, where the second annotated DICOM file includes the second annotation data and further includes an identifier that indicates the second DICOM image.

In various embodiments, the medical picture archive integration system generates parameter data for transmission to the medical picture archive system that indicates the set of unique scan categories. The medical picture archive system automatically transmits the first DICOM image to the medical picture archive integration system in response to determining that the first DICOM image compares favorably to one of the set of unique scan categories.

In various embodiments, the second memory further stores operational instructions that, when executed by the at least one second processor, cause the medical picture archive integration system to generate a natural language report data is based on the first annotation data and to transmit, via a second transmitter, the natural language report data to a report database associated with the medical picture archive integration system, where the natural language report data includes an identifier corresponding to the first DICOM image.

In various embodiments, the first memory further stores operational instructions that, when executed by the at least one first processor, cause the medical picture archive integration system to receive, via a second receiver, a natural language report corresponding to the first DICOM image from the report database. A set of patient identifying text included in the natural language report are identified. Performing the de-identification function on the first DICOM image includes searching the first DICOM image for the set of patient identifying text to identify the at least one patient identifier.

In various embodiments, the first memory is managed by a medical entity associated with the medical picture archive system. The medical picture archive integration system is located at a first geographic site corresponding to the medical entity, and the central server is located at a second geographic site. In various embodiments, the first memory is decoupled from the network to prevent the first DICOM image that includes the at least one patient identifier from being communicated via the network. In various embodiments, the medical picture archive system is a Picture Archive and Communication System (PACS) server, and the first DICOM image is received in response to a query sent to the medical picture archive system by the transmitter in accordance with a DICOM communication protocol.

FIG. 10A presents an embodiment of a de-identification system 2800. The de-identification system 2800 can be utilized to implement the de-identification system 2608 of FIGS. 8A-8F. In some embodiments, the de-identification system 2800 can be utilized by other subsystems to de-identify image data, medical report data, private fields of medical scan entries 352 such as patient identifier data 431, and/or other private fields stored in databases of the database memory device 340.

The de-identification system can be operable to receive, from at least one first entity, a medical scan and a medical report corresponding to the medical scan. A set of patient identifiers can be identified in a subset of fields of a header of the medical scan. A header anonymization function can be performed on each of the set of patient identifiers to generate a corresponding set of anonymized fields. A de-identified medical scan can be generated by replacing the subset of fields of the header of the medical scan with the corresponding set of anonymized fields.

A subset of patient identifiers of the set of patient identifiers can be identified in the medical report by searching text of the medical report for the set of patient identifiers. A text anonymization function can be performed on the subset of patient identifiers to generate corresponding anonymized placeholder text for each of the subset of patient identifiers. A de-identified medical report can be generated by replacing each of the subset of patient identifiers with the corresponding anonymized placeholder text. The de-identified medical scan and the de-identified medical report can be transmitted to a second entity via a network.

As shown in FIG. 10A, the de-identification system 2800 can include at least one receiver 2802 operable to receive medical scans, such as medical scans in a DICOM image format. The at least one receiver 2802 is further operable to receive medical reports, such as report data 449 or other reports containing natural language text diagnosing, describing, or otherwise associated the medical scans received by the de-identification system. The medical scans and report data can be received from the same or different entity, and can be received by the same or different receiver 2802 in accordance with the same or different communication protocol. For example, the medical scans can be received from the medical picture archive system 2620 of FIGS. 8A-8F and the report data can be received from the report database 2625 of FIGS. 8D-8F. In such embodiments, the receiver 2802 can be utilized to implement the receiver 2602 of FIG. 8B.

The de-identification system 2800 can further include a processing system 2804 that includes at least one processor, and a memory 2806. The memory 2806 can store operational instructions that, when executed by the processing system, cause the de-identification system to perform at least one patient identifier detection function on the received medical scan and/or the medical report to identify a set of patient identifiers in the medical scan and/or the medical report. The operational instructions, when executed by the processing system, can further cause the de-identification system to perform an anonymization function on the medical scan and/or the medical report to generate a de-identified medical scan and/or a de-identified medical report that do not include the set of patient identifiers found in performing the at least one patient identifier detection function. Generating the de-identified medical scan can include generating a de-identified header and generating de-identified image data, where the de-identified medical scan includes both the de-identified header and the de-identified image data. The memory 2806 can be isolated from Internet connectivity, and can be designated for PHI.

The de-identification system 2800 can further include at least one transmitter 2808, operable to transmit the de-identified medical scan and de-identified medical report. The de-identified medical scan and de-identified medical report can be transmitted back to the same entity from which they were received, respectively, and/or can be transmitted to a separate entity. For example, the at least one transmitter can transmit the de-identified medical scan to the de-identified image storage system 2610 of FIGS. 8A-8F and/or can transmit the de-identified medical scan to central server system 2640 via network 2630 of FIGS. 8A-8F. In such embodiments, the transmitter 2808 can be utilized to implement the interface 2655 of FIG. 8B. The receiver 2802, processing system 2804, memory 2806, and/or transmitter 2808 can be connected via bus 2810.

Some or all of the at least one patient identifier detection function and/or at least one anonymization function as discussed herein can be trained and/or implemented by one or subsystems 101 in the same fashion as other medical scan analysis functions discussed herein, can be stored in medical scan analysis function database 346 of FIG. 3, and/or can otherwise be characterized by some or all fields of a medical scan analysis function entry 356 of FIG. 5.

The de-identification system 2800 can perform separate patient identifier detection functions on the header of a medical report and/or medical scan, on the text data of the medical report, and/or on the image data of the medical scan, such as text extracted from the image data of the medical scan. Performance of each of these functions generates an output of its own set of identified patient identifiers. Combining these sets of patient identifiers yields a blacklist term set. A second pass of the header of a medical report and/or medical scan, on the text data of the medical report, and/or on the image data of the medical scan that utilizes this blacklist term set can catch any terms that were missed by the respective patient identifier detection function, and thus, the outputs of these multiple identification processes can support each other. For example, some of the data in the headers will be in a structured form and can thus be easier to reliably identify. This can be exploited and used to further anonymize these identifiers when they appear in free text header fields, report data, and/or in the image data of the medical scan. Meanwhile, unstructured text in free text header fields, report data, and/or image data of the medical scan likely includes pertinent clinical information to be preserved in the anonymization process, for example, so it can be leveraged by at least one subsystems 101 and/or so it can be leveraged in training at least one medical scan analysis function.

At least one first patient identifier detection function can include extracting the data in a subset of fields of a DICOM header, or another header or other metadata of the medical scan and/or medical report with a known type that corresponds to patient identifying data. For example, this patient identifying subset of fields can include a name field, a patient ID number field or other unique patient identifier field, a date field, a time field, an age field, an accession number field, SOP instance UID, and/or other fields that could be utilized to identify the patient and/or contain private information. A non-identifying subset of fields of the header can include hospital identifiers, machine model identifiers, and/or some or all fields of medical scan entry 352 that do not correspond to patient identifying data. The patient identifying subset of fields and the non-identifying subset of fields can be mutually exclusive and collectively exhaustive with respect to the header. The at least one patient identifier function can include generating a first set of patient identifiers by ignoring the non-identifying subset of fields and extracting the entries of the patient identifying subset of fields only. This first set of patient identifiers can be anonymized to generate a de-identified header as discussed herein.

In some embodiments, at least one second patient identifier detection function can be performed on the report data of the medical report. The at least one second patient identifier detection function can include identifying patient identifying text in the report data by performing a natural language analysis function, for example, trained by the medical scan natural language analysis system 114. For example, the at least one second patient identifier detection function can leverage the known structure of the medical report and/or context of the medical report. A second set of patient identifiers corresponding to the patient identifying text can be determined, and the second set of patient identifiers can be anonymized to generate a de-identified medical report. In some embodiments, a de-identified medical report includes clinical information, for example, because the portion of the original medical report that includes the clinical information was deemed to be free of patient identifying text and/or because the portion of the original medical report that includes the clinical information was determined to include pertinent information to be preserved.

In some embodiments, the medical report includes image data corresponding to freehand or typed text. For example the medical report can correspond to a digitized scan of original freehand text written by a radiologist or other medical professional. In such embodiments, the patient identifier detection function can first extract the text from the freehand text in the image data to generate text data before the at least one second patient identifier detection function is performed on the text of the medical report to generate the second set of patient identifiers.

In some embodiments, the at least one second patient identifier detection function can similarly be utilized to identify patient identifying text in free text fields and/or unstructured text fields of a DICOM header and/or other metadata of the medical scan and/or medical report data by performing a natural language analysis function, for example, trained by the medical scan natural language analysis system 114. A third set of patient identifiers corresponding to this patient identifying text of the free text and/or unstructured header fields can be determined, and the third set of patient identifiers can be anonymized to generate de-identified free text header field and/or unstructured header fields. In some embodiments, a de-identified free text header field and/or unstructured header field includes clinical information, for example, because the portion of the original corresponding header field that includes the clinical information was deemed to be free of patient identifying text and/or because the portion of the original corresponding header field that includes the clinical information was determined to include pertinent information to be preserved.

Patient identifiers can also be included in the image data of the medical scan itself. For example, freehand text corresponding to a patient name written on a hard copy of the medical scan before digitizing can be included in the image data, as discussed in conjunction with FIG. 10B. Other patient identifiers, such as information included on a patient wristband or other identifying information located on or within the vicinity of the patient may have been captured when the medical scan was taken, and can thus be included in the image. At least one third patient identifier detection function can include extracting text from the image data and/or detecting non-text identifiers in the image data by performing a medical scan image analysis function, for example, trained by the medical scan image analysis system 112. For example, detected text that corresponds to an image location known to include patient identifiers, detected text that corresponds to a format of a patient identifier, and/or or detected text or other image data determined to correspond to a patient identifier can be identified. The at least one third patient identifier detection function can further include identifying patient identifying text in the text extracted from the image data by performing the at least one second patient identifier detection function and/or by performing a natural language analysis function. A fourth set of patient identifiers corresponding to patient identifying text or other patient identifiers detected in the image data of the medical scan can be determined, and the fourth set of patient identifiers can be anonymized in the image data to generate de-identified image data of the medical scan as described herein. In particular, the fourth set of patient identifiers can be detected in a set of regions of image data of the medical scan, and the set of regions of the image data can be anonymized.

In some embodiments, only a subset of the patient identifier detection functions described herein are performed to generate respective sets of patient identifiers for anonymization. In some embodiments, additional patient identifier detection functions can be performed on the medical scan and/or medical report to determine additional respective sets of patient identifiers for anonymization. The sets of patient identifiers outputted by performing each patient identifier detection function can have a null or non-null intersection. The sets of patient identifiers outputted by performing each patient identifier function can have null or non-null set differences.

Cases where the sets of patient identifiers have non-null set differences can indicate that a patient identifier detected by one function may have been missed by another function. The combined set of patient identifiers, for example, generated as the union of the sets of sets of patient identifiers outputted by performing each patient identifier function, can be used to build a blacklist term set, for example, stored in memory 2806. The blacklist term set can designate the final set of terms to be anonymized. A second pass of header data, medical scans, medical reports, and/or any free text extracted from the header data, the medical scan, and/or the medical report can be performed by utilizing the blacklist term set to flag terms for anonymization that were not caught in performing the respective at least one patient identifier detection function. For example, performing the second pass can include identifying at least one patient identifier of the blacklist term set in the header, medical report, and/or image data of the medical scan. This can include by searching corresponding extracted text of the header, medical report, and/or image data for terms included in blacklist term set and/or by determining if each term in the extracted text is included in the blacklist term set.

In some embodiments, at least one patient identifier is not detected until the second pass is performed. Consider an example where a free text field of a DICOM header included a patient name that was not detected in performing a respective patient identifier detection function on the free text field of the DICOM header. However, the patient name was successfully identified in the text of the medical report in performing a patient identifier detection function on the medical report. This patient name is added to the blacklist term list, and is detected in a second pass of the free text field of the DICOM header. In response to detection in the second pass, the patient name of the free text field of the DICOM header can be anonymized accordingly to generate a de-identified free text field. Consider a further example where the patient name is included in the image data of the medical scan, but was not detected in performing a respective patient identifier detection function on the free text field of the DICOM header. In the second pass, this patient name can be detected in at least one region of image data of the medical scan by searching the image data for the blacklist term set.

In some embodiments, performing some or all of the patient identifier detection functions includes identifying a set of non-identifying terms, such as the non-identifying subset of fields of the header. In particular, the non-identifying terms can include terms identified as clinical information and/or other terms determined to be preserved. The combined set of non-identifying terms, for example, generated as the union of the sets of sets of non-identifying outputted by performing each patient identifier function, can be used to build a whitelist term set, for example, stored in memory 2806. Performing the second pass can further include identifying at least one non-identifying term of the whitelist term set in the header, medical report, and/or image data of the medical scan, and determining not to anonymize, or to otherwise ignore, the non-identifying term.

In various embodiments, some or all terms of the whitelist term set can be removed from the blacklist term set. In particular, at least one term previously identified as a patient identifier in performing one or more patient identifier detection functions is determined to be ignored and not anonymized in response to determining the term is included in the whitelist term set. This can help ensure that clinically important information is not anonymized, and is thus preserved in the de-identified medical scan and de-identified medical report.

In some embodiments, the second pass can be performed after each of the patient identifier detection functions are performed. For example, performing the anonymization function can include performing this second pass by utilizing the blacklist term set to determine the final set of terms to be anonymized. New portions of text in header fields, not previously detected in generating the first set of patient identifiers or the third set of patient identifiers, can be flagged for anonymization by determining these new portions of text correspond to terms of the blacklist term set. New portions of text the medical report, not previously detected in generating in the second set of patient identifiers, can be flagged for anonymization by determining these new portions of text correspond to terms of the blacklist term set. New regions of the image data of the medical scan, not previously detected in generating the fourth set of patient identifiers, can be flagged for anonymization by determining these new portions of text correspond to terms of the blacklist term set.

In some embodiments, the blacklist term set is built as each patient identifier detection function is performed, and performance of subsequent patient identifier detection functions includes utilizing the current blacklist term set. For example, performing the second patient identifier detection function can include identifying a first subset of the blacklist term set in the medical report by searching the text of the medical report for the blacklist term set and/or by determining if each term in the text of the medical report is included in the blacklist term set. Performing the second patient identifier detection function can further include identifying at least one term in the medical report that is included in the whitelist term set, and determining to ignore the term in response. The first subset can be anonymized to generate the de-identified medical report as discussed herein. New patient identifiers not already found can be appended to the blacklist term set, and the updated blacklist term set can be applied to perform a second search of the header and/or image data of the medical scan, and at least one of the new patient identifiers can be identified in the header in the second search of the header and/or in the image data in a second search of the image data. These newly identified patient identifiers in the header and/or image data are anonymized in generating the de-identified medical scan.

As another example, a second subset of the blacklist term set can be detected in a set of regions of image data of the medical scan by performing the medical scan image analysis function on image data of the medical scan, where the image analysis function includes searching the image data for the set of patient identifiers. For example, the medical scan image analysis function can include searching the image data for text, and the second subset can include detected text that matches one or more terms of the blacklist term set. In some embodiments, detected text that matches one or more terms of the whitelist term set can be ignored. The second subset can be anonymized to generate de-identified image data as discussed herein. New patient identifiers that are detected can be appended to the blacklist term set, and the updated blacklist term set can be applied to perform a second search of the header and/or metadata of the medical scan, and/or can be applied to perform a second search of the medical report. At least one of the new patient identifiers can be identified in the header as a result of performing the second search of the header and/or at least one of the new patient identifiers can be identified medical report as a result of performing the second search of the medical report. These newly identified patient identifiers can be anonymized in the header along with the originally identified blacklist term set in generating the de-identified header, and/or can be anonymized in the medical report along with the originally identified first subset in generating the de-identified medical report.

In some embodiments, the memory 2806 further stores a global blacklist, for example, that includes a vast set of known patient identifying terms. In some embodiments, the global blacklist is also utilized by at least one patient identifier detection function and/or in performing the second pass to determine patient identifying terms for anonymization. In some embodiments, the blacklist term set generated for a particular medical scan and corresponding medical report can be appended to the global blacklist for use in performing the second pass and/or in detecting patient identifiers in subsequently received medical scans and/or medical reports.

Alternatively or in addition, the memory 2806 can further store a global whitelist, for example, that includes a vast set of terms that can be ignored. In particular, the global whitelist can include clinical terms and/or other terms that are deemed beneficial to preserve that do not correspond to patient identifying information. In some embodiments, the global whitelist is utilized by at least one patient identifier detection function and/or in performing the second pass to determine terms to ignore in the header, image data, and/or medical report. In some embodiments, the whitelist term set generated for a particular medical scan and corresponding medical report can be appended to the global whitelist for use in performing the second pass and/or in ignoring terms in subsequently received medical scans and/or medical reports.

Alternatively or in addition, the memory 2806 can further store a global graylist, for example, that includes ambiguous terms that could be patient identifying terms in some contexts, but non-identifying terms in other contexts. For example, “Parkinson” could correspond to patient identifying data if part of a patient name such as “John Parkinson”, but could correspond to non-patient identifying data meant to be ignored and preserved in the de-identified medical report and/or de-identified medical scan if part of a diagnosis term such as “Parkinson's disease.” In some embodiments, the global graylist is also utilized in performing the second pass and/or in performing at least one patient identifier detection function to determine that a term is included in the graylist, and to further determine whether the term should be added to the blacklist term set for anonymization or whitelist term set to be ignored by leveraging context of accompanying text, by leveraging known data types of a header field from which the term was extracted, by leveraging known structure of the term, by leveraging known data types of a location of the image data from which the term was extracted, and/or by leveraging other contextual information. In some embodiments, the graylist term set can be updated based on blacklist and/or whitelist term sets for a particular medical scan and corresponding medical report.

In some embodiments, the at least one anonymization function includes a fiducial replacement function. For example, some or all of the blacklist term set can be replaced with a corresponding, global fiducial in the header, report data, and/or image data. In some embodiments, the global fiducial can be selected from a set of global fiducials based on a type of the corresponding patient identifier. Each patient identifier detected in the header and/or medical report can be replaced with a corresponding one of the set of global text fiducials. Each patient identifiers detected in the image data can be replaced with a corresponding one of the set of global image fiducials. For example, one or more global image fiducials can overlay pixels of regions of the image data that include the identifying patient data, to obfuscate the identifying patient data in the de-identified image data.

The global text fiducials and/or global image fiducials can be recognizable by inference functions and/or training functions, for example, where the global text fiducials and global image fiducials are ignored when processed in a training step to train an inference function and/or are ignored in an inference step when processed by an inference function. Furthermore, the global text fiducials and/or global image fiducials can be recognizable by a human viewing the header, medical report, and/or image data. For example, a radiologist or other medical professional, upon viewing a header, medical report, and/or image data, can clearly identify the location of a patient identifier that was replaced by the fiducial and/or can identify the type of patient identifier that was replaced by the fiducial.

As an example, the name “John Smith” can be replaced in a header and/or medical report with the text “% PATIENT NAME %”, where the text “% PATIENT NAME %” is a global fiducial for name types of the header and/or the text of medical reports. The training step and/or inference step of medical scan natural language analysis functions can recognize and ignore text that matches “% PATIENT NAME %” automatically.

FIG. 10B illustrates an example of anonymizing patient identifiers in image data of a medical scan. In this example, the name “John Smith” and the date “May 4, 2010” is detected as freehand text in the original image data of a medical scan. The regions of the image data that include the patient identifiers can each be replaced by global fiducial in the shape of a rectangular bar, or any other shape. As shown in FIG. 10B, a first region corresponding to the location of “John Smith” in the original image data is replaced by fiducial 2820 in the de-identified image data, and a second region corresponding to the location of “May 4, 2010” in the original image data is replaced by fiducial 2822 in the de-identified image data. The size, shape, and/or location of each global visual fiducial can be automatically determined based on the size, shape, and/or location of the region that includes the patient identifier to minimize the amount of the image data that is obfuscated, while still ensuring the entirety of the text is covered. While not depicted in FIG. 10B, the fiducial can be of a particular color, for example, where pixels of the particular color are automatically recognized by the training step and/or inference step of medical scan image analysis functions to indicate that the corresponding region be ignored, and/or where the particular color is not included in the original medical scan and/or is known to not be included in any medical scans. The fiducial can include text recognizable to human inspection such as “% PATIENT NAME” and “% DATE” as depicted in FIG. 10B, and/or can include a QR code, logo, or other unique symbol recognizable to human inspection and/or automatically recognizable by the training step and/or inference step of medical scan image analysis functions to indicate that the corresponding region be ignored.

In some embodiments, other anonymization functions can be performed on different ones of the patient identifying subset of fields to generate the de-identified header, de-identified report data, and/or de-identified image data. For example, based on the type of identifying data of each field of the header, different types of header anonymization functions and/or text anonymization functions can be selected and utilized on the header fields, text of the report, and/or text extracted from the image data. A set of anonymization functions can include a shift function, for example, utilized to offset a date, time or other temporal data by a determined amount to preserve absolute time difference and/or to preserve relative order over multiple medical scans and/or medical reports of a single patient. FIG. 10B depicts an example where the shift function is performed on the date detected in the image data to generate fiducial 2822, where the determined amount is 10 years and 1 month. The determined amount can be determined by the de-identification system randomly and/or pseudo-randomly for each patient and/or for each medical scan and corresponding medical report, ensuring the original date cannot be recovered by utilizing a known offset. In various embodiments, other medical scans and/or medical reports are fetched for the same patient by utilizing a patient ID number or other unique patient identifier of the header. These medial scans and reports can be anonymized as well, where the dates and/or times detected in these medical scans and/or medical reports offset by the same determined amount, randomized or pseudo-randomized for particular patient ID number, for example, based on performing a hash function on the patient ID number.

The set of anonymization functions can include at least one hash function, for example utilized to hash a unique patient ID such as a patient ID number, accession number, and/or SOP instance UID of the header and/or text. In some embodiments, the hashed SOP instance UID, accession number, and/or patient ID number are prepended with a unique identifier, stored in a database of the memory 2806 and/or shared with the entities to which the de-identified medical scans and/or medical reports are transmitted, so that de-identified medical scans and their corresponding de-identified medical reports can be linked and retrieved retroactively. Similarly, longitudinal data can be preserved as multiple medical scans and/or medical reports of the same patient will be assigned the same hashed patient ID.

The set of anonymization functions can further include at least one manipulator function for some types of patient identifiers. Some values of header fields and/or report text that would normally not be considered private information can be considered identifying patient data if they correspond to an outlier value or other rare value that could then be utilized to identify the corresponding patient from a very small subset of possible options. For example, a patient age over 89 could be utilized to determine the identity of the patient, for example, if there are very few patients over the age of 89. To prevent such cases, in response to determining that a patient identifier corresponds to an outlier value and/or in response to determining that a patient identifier compares unfavorably to a normal-range threshold value, the patient identifier can be capped at the normal-range threshold value or can otherwise be manipulated. For example, a normal-range threshold value corresponding to age can be set at 89, and generating a de-identified patient age can include capping patient ages that are higher than 89 at 89 and/or can include keeping the same value for patient ages that are less than or equal to 89.

In some embodiments, the de-identified header data is utilized to replace the corresponding first subset of patient identifiers detected in the medical report with text of the de-identified header fields. In other embodiments, a set of text anonymization functions includes a global text fiducial replacement function, shift function, a hash function, and/or manipulator functions that anonymize the corresponding types of patient identifiers in the medical report separately.

In some embodiments where the image data of a medical scan includes an anatomical region corresponding to a patient's head, the image data may include an identifying facial structure and/or facial features that could be utilized to determine the patient's identity. For example, a database of facial images, mapped to a corresponding plurality of people including the patient, could be searched and a facial recognition function could be utilized to identify the patient in the database. Thus, facial structure included in the image data can be considered patient identifying data.

To prevent this problem and maintain patient privacy, the de-identification system can further be implemented to perform facial obfuscation for facial structure detected in medical scans. At least one region of the image data that includes identifying facial structure can be determined by utilizing a medical image analysis function. For example, the medical image analysis function can include a facial detection function that determines the regions of the image data that include identifying facial structure based on searching the image data for pixels with a density value that corresponds to facial skin, facial bone structure, or other density of an anatomical mass type that corresponds to identifying facial structure, and the facial obfuscation function can be performed on the identified pixels. Alternatively or in addition, the facial detection function can determine the region based on identifying at least one shape in the image data that corresponds to a facial structure.

The image obfuscation function can include a facial structure obfuscation function performed on the medical scan to generate de-identified image data that does not include identifying facial structure. For example, the facial structure obfuscation function can mask, scramble, replace with a fiducial, or otherwise obfuscate the pixels of the region identified by the facial detection function. In some embodiments, the facial structure obfuscation function can perform a one-way function on the region that preserves abnormalities of the corresponding portions of the image, such as nose fractures or facial skin legions, while still obfuscating the identifying facial structure such that the patient is not identifiable. For example, the pixels of the identifying facial structure can be altered such that they converge towards a fixed, generic facial structure. In some embodiments, a plurality of facial structure image data of a plurality of patients can be utilized to generate the generic facial structure, for example, corresponding to an average or other combination of the plurality of faces. For example, the pixels of the generic facial structure can be averaged with, superimposed upon, or otherwise combined with the pixels of the region of the image data identified by the facial detection function in generating the de-identified image data.

In some embodiments, a hash function can be performed on an average of the generic facial structure and the identified facial structure of the image data so that the generic facial structure cannot be utilized in conjunction with the resulting data of the de-identified image data to reproduce the original, identifying facial structure. In such embodiments, the hash function can alter the pixel values while still preserving abnormalities. In some embodiments, a plurality of random, generic facial structures can be generated by utilizing the plurality of facial structure image data, for example, where each if the plurality of facial structure image data are assigned a random or pseudo-random weight in an averaging function utilized to create the generic facial structure, where a new, random or pseudo-random set of weights are generated each time the facial structure obfuscation function is utilized to create a new, generic facial structure to be averaged with the identified facial structure in creating the de-identified image data to ensure the original identifying facial structure cannot be extracted from the resulting de-identified image data.

While facial obfuscation is described herein, similar techniques can be applied in a similar fashion to other anatomical regions that are determined to include patient identifiers and/or to other anatomical regions that can be utilized to extract patient identifying information if not anonymized.

In some embodiments, the at least one receiver 2802 is included in at least one transceiver, for example, enabling bidirectional communication between the medical picture archive system 2620 and/or the report database 2625. In such embodiments, the de-identification system 2800 can generate queries to the medical picture archive system 2620 and/or the report database 2625 for particular medical scans and/or medical reports, respectively. In particular, if the medical scan and medical report are stored and/or managed by separate memories and/or separate entities, they may not be received at the same time. However, a linking identifier, such as DICOM identifiers in headers or metadata of the medical scan and/or medical report, such accession number, patient ID number, SOP instance UID, or other linking identifier that maps the medical scan to the medical report can be utilized to fetch a medical report corresponding to a received medical scan and/or to fetch a medical scan corresponding to a received medical report via a query sent utilizing the at least one transceiver. For example, in response to receiving the medical scan from the medical picture archive system 2620, the de-identification system can extract a linking identifier from a DICOM header of the medical scan, and can query the report database 2625 for the corresponding medical report by indicating the linking identifier in the query. Conversely, in response to receiving the medical report from the report database 2625, the de-identification system can extract the linking identifier from a header, metadata, and/or text body of the medical report, and can query the medical picture archive system 2620 for the corresponding medical scan by indicating the linking identifier in the query. In some embodiments, a mapping of de-identified medical scans to original medical scans, and/or a mapping of de-identified medical reports to original medical reports can be stored in memory 2806. In some embodiments, linking identifiers such as patient ID numbers can be utilized to fetch additional medical scans, additional medical reports, or other longitudinal data corresponding to the same patient.

FIG. 11 presents a flowchart illustrating a method for execution by a de-identification system 2800 that stores executional instructions that, when executed by at least one processor, cause the de-identification to perform the steps below.

Step 2902 includes receiving from a first entity, via a receiver, a first medical scan and a medical report corresponding to the medical scan. Step 2904 includes identifying a set of patient identifiers in a subset of fields of a first header of the first medical scan. Step 2906 includes performing a header anonymization function on each of the set of patient identifiers to generate a corresponding set of anonymized fields. Step 2908 includes generating a first de-identified medical scan by replacing the subset of fields of the first header of the first medical scan with the corresponding set of anonymized fields. Step 2910 includes identifying a first subset of patient identifiers of the set of patient identifiers in the medical report by searching text of the medical report for the set of patient identifiers. Step 2912 includes performing a text anonymization function on the first subset of patient identifiers to generate corresponding anonymized placeholder text for each of the first subset of patient identifiers. Step 2914 includes generating a de-identified medical report by replacing each of the first subset of patient identifiers with the corresponding anonymized placeholder text. Step 2916 includes transmitting, via a transmitter, the de-identified first medical scan and the de-identified medical report to a second entity via a network.

In various embodiments, the medical scan is received from a Picture Archive and Communication System (PACS), where the medical report is received from a Radiology Information System (RIS), and where the first de-identified medical scan and the de-identified medical report are transmitted to a central server that is not affiliated with the PACS or the MS. In various embodiments, first medical scan and the medical report are stored in a first memory for processing. The first memory is decoupled from the network to prevent the set of patient identifiers from being communicated via the network. The first de-identified medical scan and the de-identified medical report are stored in a second memory that is separate from the first memory. The first de-identified medical scan and the de-identified medical report are fetched from the second memory for transmission to the second entity.

In various embodiments, the header anonymization function performed on each of the set of patient identifiers is selected from a plurality of header anonymization functions based on one of a plurality of identifier types of the corresponding one of the subset of fields. In various embodiments, the plurality of identifier types includes a date type. A shift function corresponding to the date type is performed on a first date of the first header to generate the first de-identified medical scan, where the shift function includes offsetting the first date by a determined amount. A second medical scan that includes a second header is received, via the receiver. A unique patient ID of the first header matches a unique patient ID of the second header. The shift function is performed on a second date of the second header by offsetting the second date by the determined amount to generate a second de-identified medical scan. The second de-identified medical scan is transmitted to the second entity via the network.

In various embodiments, the plurality of identifier types includes a unique patient ID type. A hash function corresponding the unique patient ID type is performed on the unique patient ID of the first header to generate the first de-identified medical scan. The hash function is performed on the unique patient ID of the second header to generate the second de-identified medical scan. An anonymized unique patient ID field of the first de-identified medical scan matches an anonymized unique patient ID field of the second de-identified medical scan as a result of the unique patient ID of the first header matching the unique patient ID of the second header.

In various embodiments, the plurality of identifier types includes a linking identifier type that maps the medical scan to the medical report. A hash function corresponding to the linking identifier type is performed on a linking identifier of the first header to generate a hashed linking identifier. A linking identifier field of the first de-identified medical scan includes the hashed linking identifier. Performing the text anonymization function on the first subset of patient identifiers includes determining one of the first subset of patient identifiers corresponds to linking identifier text and performing the hash function on the one of the first subset of patient identifiers to generate the hashed linking identifier, where the de-identified medical report includes the hashed linking identifier.

In various embodiments, a second subset of patient identifiers of the set of patient identifiers is identified in a set of regions of image data of the medical scan by performing an image analysis function on image data of the medical scan. The image analysis function includes searching the image data for the set of patient identifiers. An identifier type is determined for each of the second subset of patient identifiers. One of a plurality of image fiducials is selected for each of the second subset of patient identifiers based on the identifier type. De-identified image data is generated, where a set of regions of the de-identified image data, corresponding to the set of regions of the image data, includes the one of the plurality of image fiducials to obfuscate each of the second subset of patient identifiers. Generating the first de-identified medical scan further includes replacing the image data of the medical scan with the de-identified image data.

In various embodiments, a new patient identifier is identified in the medical report by performing a natural language analysis function on the medical report, where new patient identifier is not included in the set of patient identifiers. The set of patient identifiers is updated to include the new patient identifier prior to searching the image data of the medical scan for the set of patient identifiers, and the second subset of patient identifiers includes the new patient identifier.

In various embodiments, the memory further stores a global identifier blacklist. The natural language analysis function includes searching the medical report for a plurality of terms included in the global identifier blacklist to identify the new patient identifier. In various embodiments, the de-identification system determines that the global identifier blacklist does not include one of the set of patient identifiers, and the global identifier blacklist is updated to include the one of the set of patient identifiers.

In various embodiments, performing the image analysis function further includes identifying a new patient identifier in the image data, where new patient identifier is not included in the set of patient identifiers. Identifying text is extracted from a region of the image data corresponding to the new patient identifier. The new patient identifier is identified in the medical report by searching text of the medical report for the identifying text. The text anonymization function is performed on new patient identifier to generate anonymized placeholder text for the new patient identifier. Generating the de-identified medical report further includes replacing the identifying text with the anonymized placeholder text for the new patient identifier.

In various embodiments, generating the de-identified image data further includes detecting an identifying facial structure in the image data of the medical scan. Generating the de-identified image data includes performing a facial structure obfuscation function on the image data, and where the de-identified image data does not include the identifying facial structure.

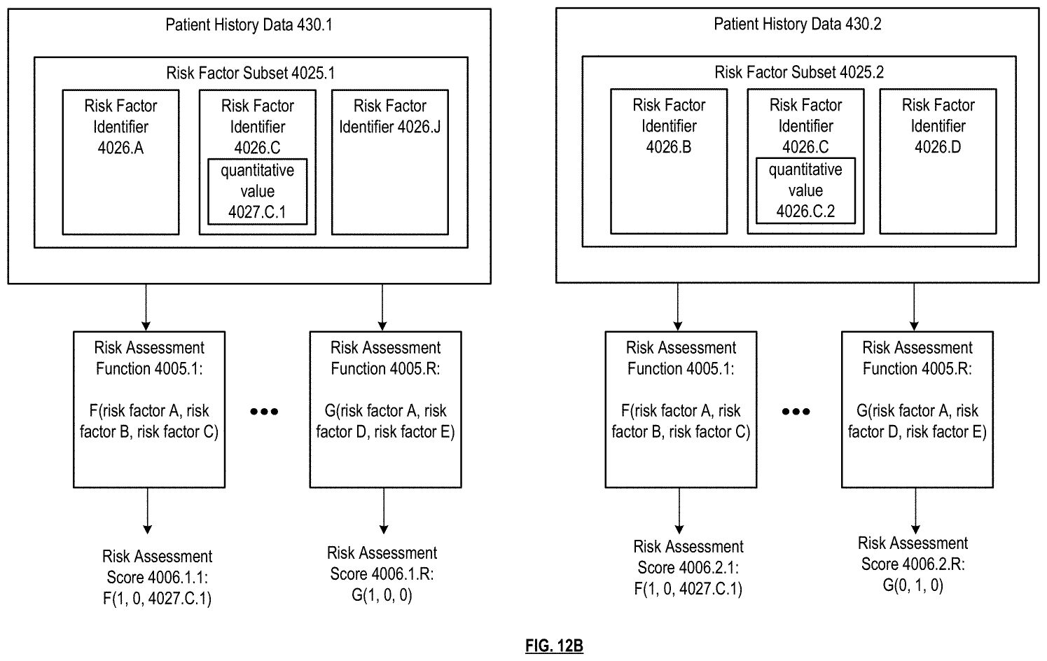

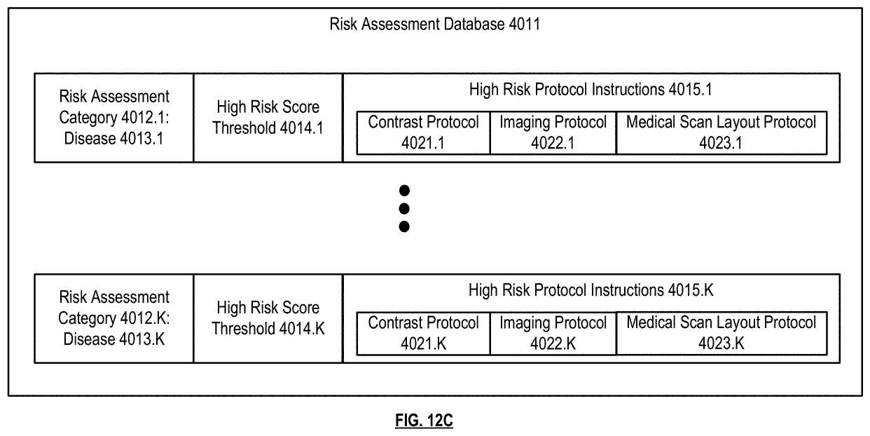

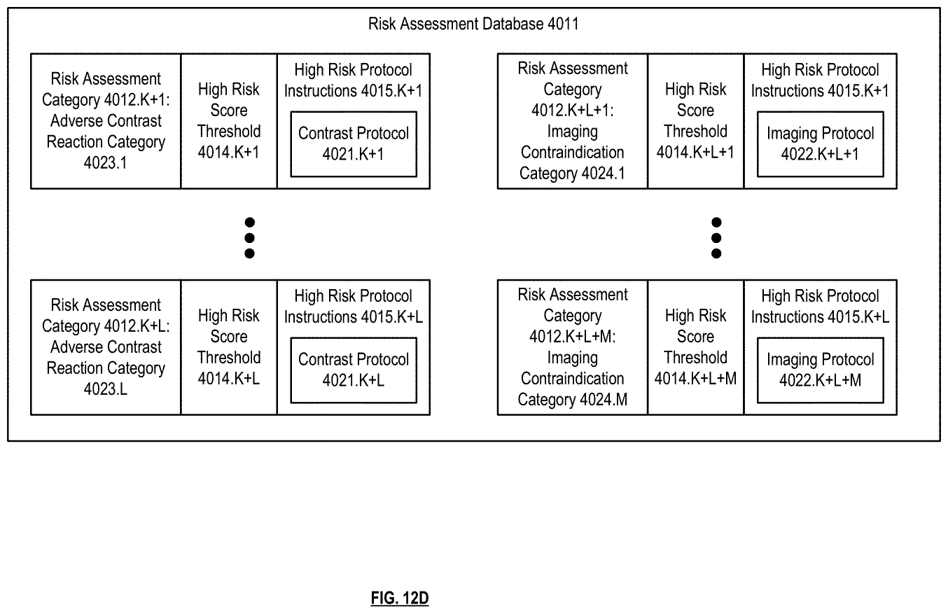

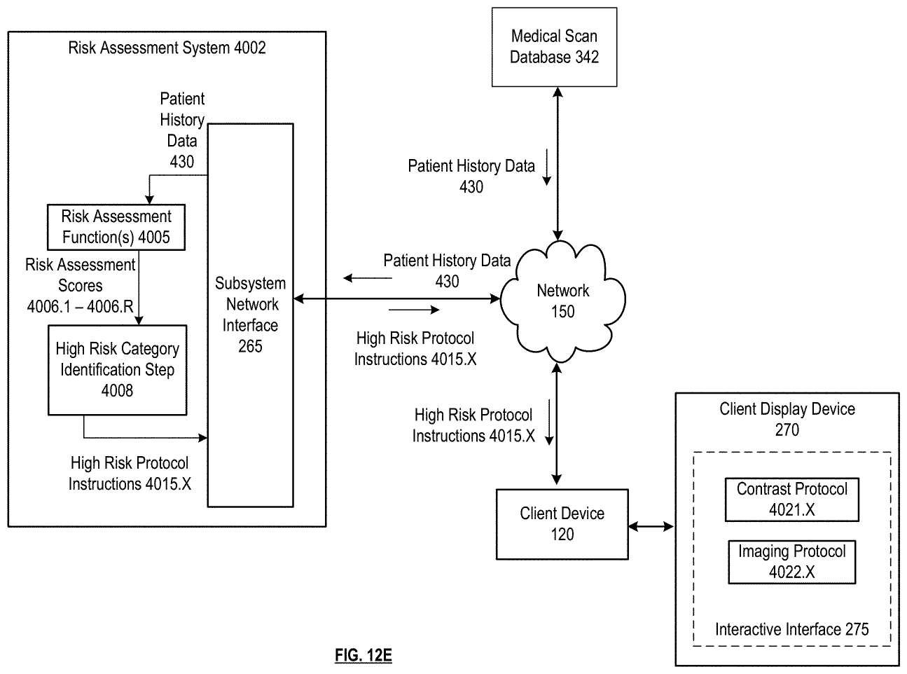

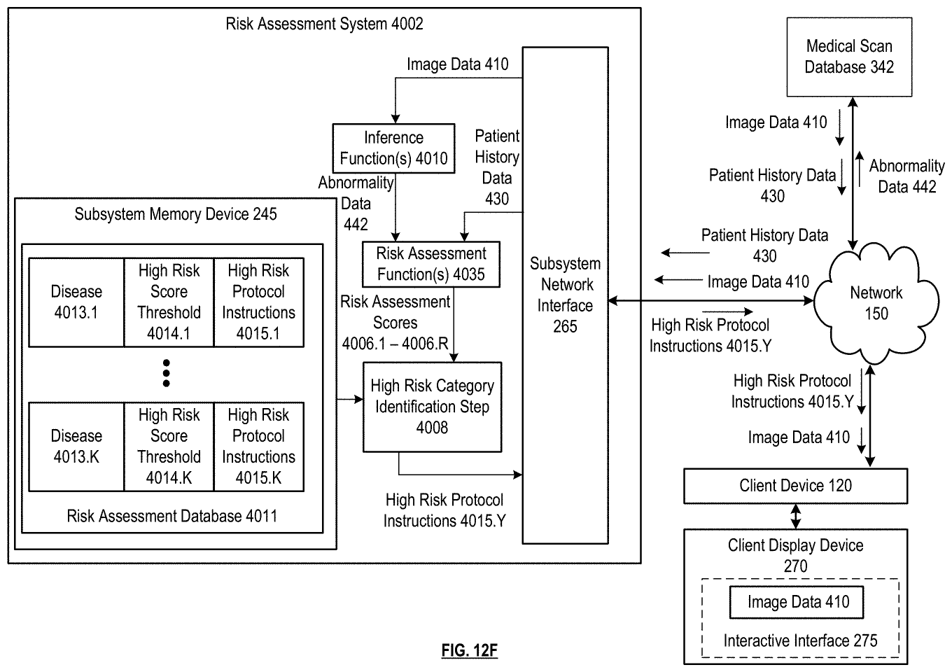

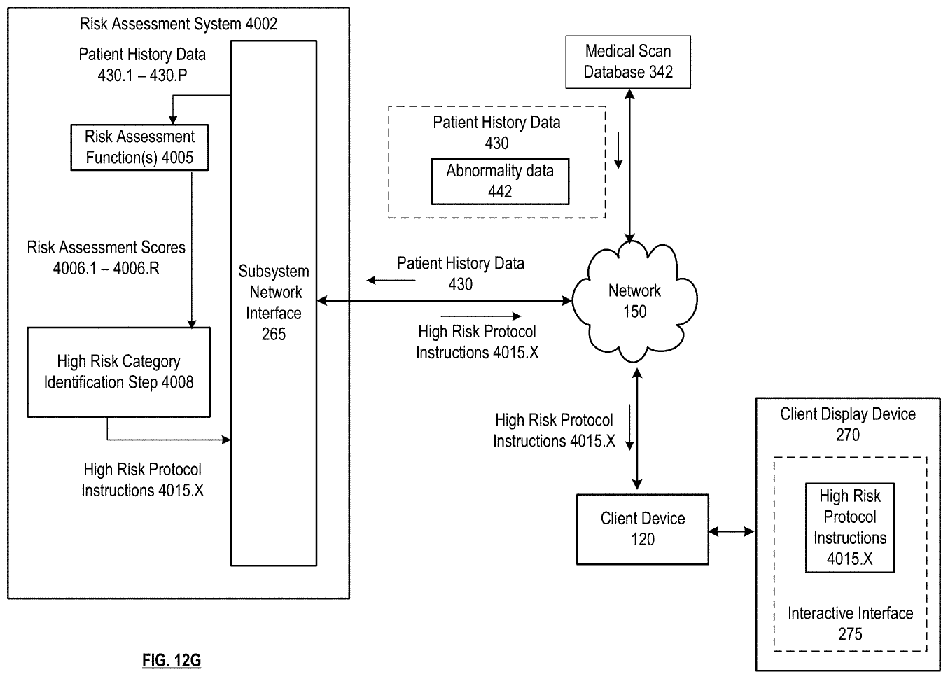

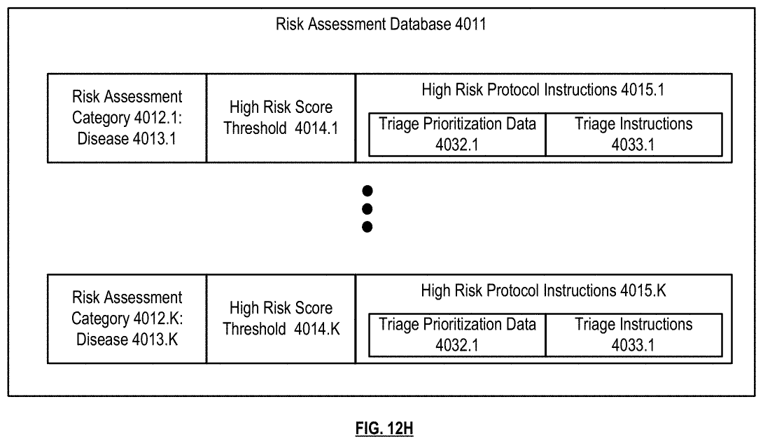

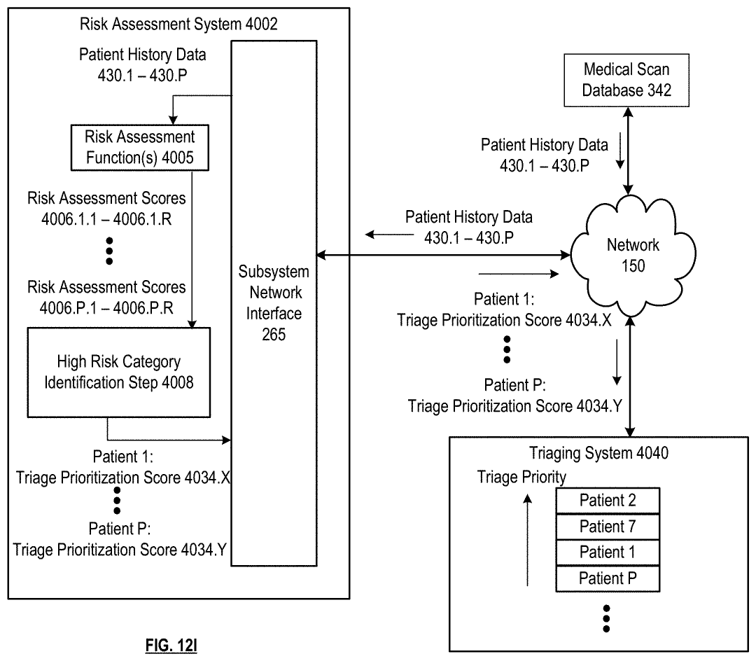

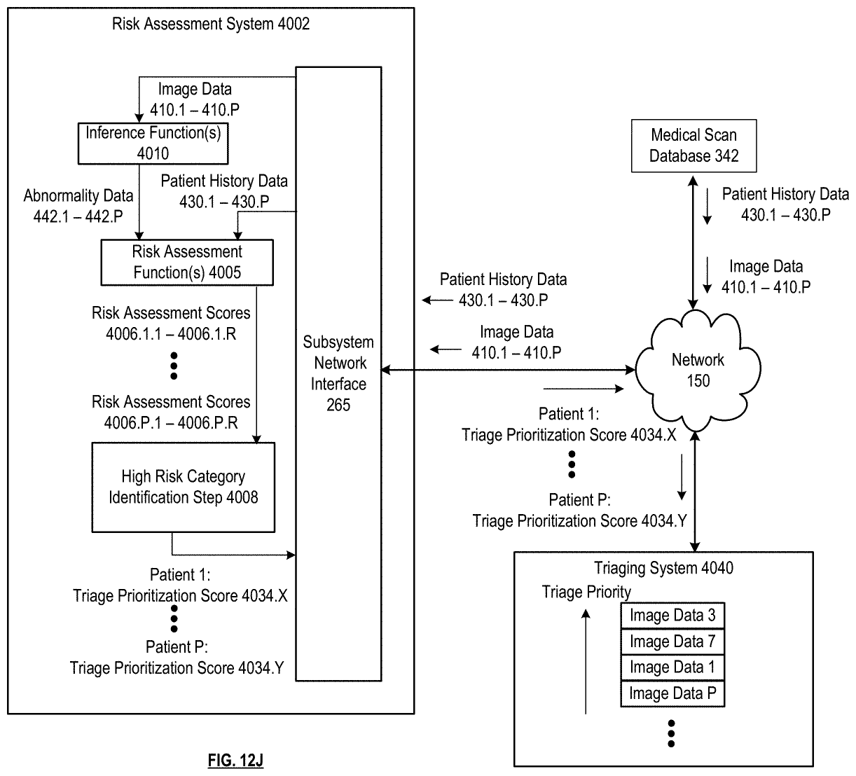

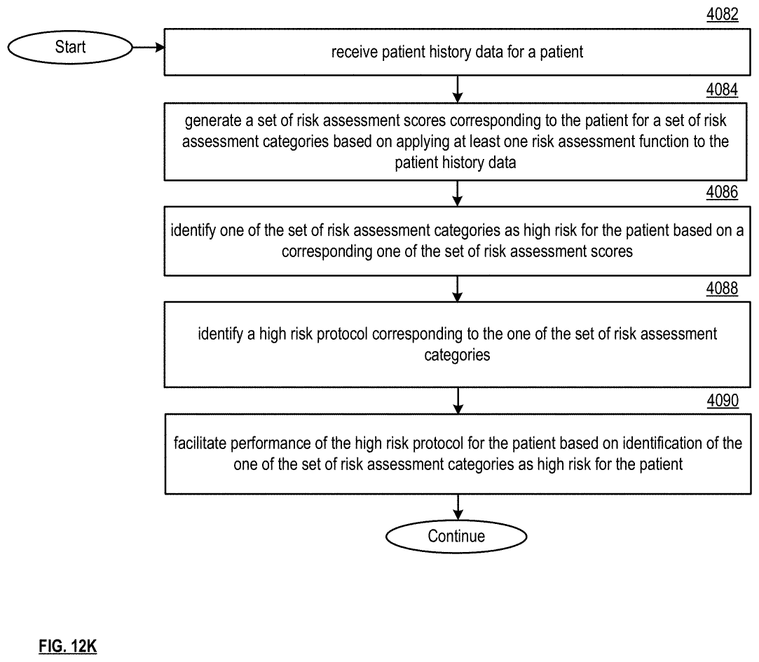

FIGS. 12A-12J present embodiments of a risk assessment system 4002. The risk assessment system 4002 is operable to receive and utilize patient history data 430 of a patient to generate a risk assessment score 4006 corresponding to the patient for each of a set of risk assessment categories 4012. One or more risk assessment scores may indicate the corresponding risk assessment category is a high risk category for the patient. These one or more high risk categories can have corresponding protocols, and high risk protocol instructions for the one or more high risk categories can be sent to a client device for viewing by a medical professional and/or can be executed automatically by a processing module of the risk assessment system 4002 and/or by another subsystem 101. The automatic determination of high risk conditions and the initiation of corresponding protocols improves the technology of patient care for medical conditions by automatically ensuring that patients receive care and/or automatically recommending particular medical protocols be utilized in accordance with inherent risks that may be detected for a particular patient based on their medical history.

Risk assessment system 4002 can communicate bi-directionally, via network 150, with the medical scan database 342 and/or other databases of the database storage system 140, with one or more client devices 120, and/or, while not shown in FIG. 12A, one or more subsystems 101 of FIG. 1. In some embodiments, the risk assessment system 4002 is an additional subsystem 101 of the medical scan processing system 100, implemented by utilizing the subsystem memory device 245, subsystem processing device 235, and/or subsystem network interface 265 of FIG. 2B. In some embodiments, some or all of the risk assessment system 4002 is implemented by utilizing other subsystems 101 and/or is operable to perform functions or other operations described in conjunction with one or more other subsystems 101.

As illustrated in FIG. 12A, the risk assessment system 4002 can be operable to receive patient history data 430. The patient history data 430 can be received from medical scan database 342 and/or can be received from another database and/or memory that stores patient history. In some cases, the patient history data 430 is received directly from a client device 120 based on user input entered by the patient and/or a medical professional associated with the patient indicating some or all of the patient history data 430. In some cases, the patient history data 430 is extracted from prior report data 449 and/or prior medical scans as one or more abnormality data 442.

The patient history data 430 can include some or all of the patient identifier data 431, risk factor data 432, longitudinal data 433, and/or longitudinal quality score 434 as illustrated in FIG. 4A. In some cases, the received patient history data only includes risk factor data 432 and/or patient identifier data 431. The patient history data 430 can otherwise indicate one or more risk factors denoted based on one or more of: previous medical history, family medical history, smoking and/or drug habits, pack years corresponding to tobacco use, environmental exposures and/or working conditions, travel history, diet, exercise habits, recreational activities, current and/or past patient symptoms, allergies, and/or other medical risk factors.

The one or more risk factors indicated the patient history data 430 can indicate risk factors associated with one or more particular diseases and/or other medical conditions. For example, a history of smoking and/or an occupation of working in a shipyard can be indicated in patient history data 430, and can correspond to risk factors associated with lung cancer. The one or more risk factors indicated in the patient history data 430 can alternatively or additionally indicate risk factors associated with one or more contrasts that could be administered to the patient in capturing medical scans, such as risk factors indicating allergic reactions to one or more contrasts and/or such as risk factors indicating history of anaphylactic shock.

In some cases, the patient history data 430 can be standardized by the risk assessment system 4002 and/or by another subsystem 101. For example, different risk factors included in a given patient history data 430 for a given patient can be denoted via standardized codes and/or labels, such as medical codes 447 and/or custom labels utilized by the risk assessment system to easily identify a known set of risk factors in each incoming patient history data 430. In some cases, a natural language processing function can be trained and performed to extract and/or assign a set of standardized codes indicating a set of risk factors for a given patient from medical reports, natural language text, and/or speech entered by a patient and/or medical professional and/or patient via user input, for example, by utilizing the natural language medical scan analysis system 114. An example embodiment of the structure of patient history data 430 is discussed in conjunction with FIG. 12B.

The set of risk factors indicated in given patient history data 430 can be utilized as input to one or more risk assessment functions 4005 that, when performed upon patient history data 430, generates a set of one or more risk assessment scores 4006.1-4006.R for the patient, where each risk assessment score 4006 corresponds to one of a set of one or more risk assessment categories 4012.1-4012.R. Some or all risk assessment scores 4006 can indicate a probability value or other numerical value denoting a level of risk that the patient has and/or will contract a condition associated with the corresponding risk assessment category 4012. Some or all risk assessment scores 4006 can indicate a binary value denoting whether or not the patient is at risk for having and/or contracting the condition associated with the corresponding risk assessment category 4012. In some embodiments, a single risk assessment score 4006 is alternatively generated that corresponding to a single, global risk category denoting whether the patient is at risk for health conditions in general.

In some embodiments, each risk assessment category 4012 has its own corresponding risk assessment function 4005, where a set of set of risk assessment functions 4005.1-4005.R are each performed to generate the set of risk assessment scores 4006.1-4006.R. In other embodiments, one or more risk assessment functions 4005 are performed that generate multiple risk assessment scores 4006 corresponding to multiple ones of the set of risk assessment scores 4006.1-4006. The one or more risk assessment functions 4005 can be retrieved from local memory, can be received via network interface 265, can be configured via user input, can be trained by applying one or more machine learning models based on a training set of patient history data and final diagnosis data for a set of patients, and/or can be stored in function database 346. Example embodiments of risk assessment functions 4005 are discussed in conjunction with FIG. 12B.

A high risk category identification step can be performed based on the risk assessment scores 4006.1-4006.R to identify one or more risk assessment categories deemed as “high risk” for the patient that necessitate action be taken. In this example, a particular risk assessment category 4012.X is identified as the single high risk category, for example, based on the corresponding risk assessment score 4006.X. In some cases, multiple risk assessment categories are identified as high risk categories for the corresponding patient. In some cases, no risk assessment categories are identified as high risk categories for the corresponding patient. In cases where a single risk assessment score 4006 is generated for a single global risk category, the single risk assessment score 4006 is utilized to determine whether the patient is a “high risk” in general.

In some embodiments, as illustrated in FIG. 12A, a risk assessment database 4011 can store a set of high risk score thresholds 4014.1-4014.R corresponding to the set of risk assessment categories 4014.1-40124.R. The risk assessment database 4011 can be stored in memory of the risk assessment system 4002 as illustrated in FIG. 12A. In other embodiments, the risk assessment database 4011 is stored in other memory accessible by the risk assessment system 4002, for example, via the network. In some embodiments, the risk assessment database 4011 is stored as an additional database stored by database memory device 340.

In such embodiments, for example, where the risk assessment functions 4005 are configured to generate risk assessment scores with increasing values indicating higher levels of risk, risk assessment scores 4006 with values that meet, exceed, and/or otherwise compare favorably to the high risk score threshold 4014 of the corresponding risk assessment category are deemed as “high risk”. Risk assessment scores 4006 with values that do not meet, exceed, and/or otherwise compare favorably to the high risk score threshold 4014 of the corresponding risk assessment category are not deemed as “high risk”. In other embodiments, for example, where the risk assessment functions 4005 are configured to generate risk assessment scores with decreasing values indicating higher levels of risk, only risk assessment scores 4006 with values that meet, fall below, and/or otherwise compare favorably to the high risk score threshold 4014 of the corresponding risk assessment category are deemed as “high risk”. In other embodiments, for example, where the risk assessment functions 4005 are configured to generate a binary output indicating whether the patient is at risk for the corresponding risk assessment category, only risk assessment scores 4006 with binary values indicating the risk are deemed as “high risk.”

Different high risk score thresholds can optionally be configured for different risk assessment categories. For example, risk assessment categories corresponding to more severe diseases and/or more time sensitive and/or conditions can have looser high risk score thresholds than that of less severe and/or less time sensitive diseases and/or conditions to ensure that patients with risk assessment scores indicating lower risks of having and/or contracting the severe disease and/or condition are treated more cautiously with appropriate protocols initiated accordingly. In other embodiments, a same high risk score thresholds can optionally be applied to risk assessment scores of some or all risk assessment categories.

In some embodiments, the risk assessment scores 4006.1-4006.R are optionally ranked by their value in increasing or decreasing order, where one or more risk assessment categories 4012 with highest ranked, highly ranked, and/or otherwise favorably ranked risk assessment scores 4006 in the ranking correspond to “high risk”. In some embodiments, first risk assessment category 4012 with a first risk assessment score 4006 that is ranked higher than a second risk assessment score 4006 of a second risk assessment category 4012 is determined to be a higher risk than the second risk assessment category 4012 for the given patient.

Once the one or more high risk categories are identified, the risk assessment system 4002 can initiate and/or facilitate performance of one or more protocols corresponding to the one or more high risk categories. As illustrated in FIG. 12A, the risk assessment database 4011 can store a set of high risk protocol instructions to be performed when the corresponding risk assessment category is identified as high risk for the corresponding patient. In some embodiments, the high risk protocol instructions are preset and/or are the same for all patients with a corresponding risk assessment score for the corresponding category that compares favorably to the high risk score threshold and/or is otherwise deemed as high risk. In other embodiments, the high risk protocol instructions are determined and/or selected as function of the risk assessment score for the corresponding category patients, where a more extreme high risk protocol is initiated for patients with a first risk assessment score for the corresponding category indicating they are at incredibly high risk, while more extreme high risk protocol is initiated for patients with a second risk assessment score for the corresponding category indicating they are at moderately high risk, for example based on the first risk assessment score being higher than or otherwise indicating higher risk than the second risk assessment score.

As illustrated in FIG. 12A, the high risk protocol instructions 4015.X can be transmitted to the client device 120 for execution and/or for display to a medical professional. Examples of executing and/or communicating the high risk protocol instructions are discussed in further detail in conjunction with FIGS. 12E-12J.

FIG. 12B illustrates an example embodiment of how risk assessment scores 4006.1-4006.R of FIG. 12A are generated for two different patient history data 430.1 and 430.2. Patient history data 430.1, corresponding to a first patient, has a risk factor subset 4025.1 indicating the presence of three risk factors A, C, and J, identified with corresponding risk factor identifiers 4026.A, 4026,C, and 4026.J, which can be implemented utilizing as standardized codes and/or labels identifying each of the risk factors A, C, and J. Patient history data 430.2, corresponding to a second patient, has a risk factor subset 4025.2 indicating the presence of three risk factors B, C, and D, identified with corresponding risk factor identifiers 4026.B, 4026.C, and 4026.D. In this example, other possible risk factors, such as risk factors E, F, G, H, and I, are not identified in either patient's patient history data 430. In some cases, some or all of these other risk factors E, F, G, H, and I can be denoted in risk factor subset 4025 as either being known to not be present or being unknown as to whether or not the risk factor is not present.

Some risk factors can correspond to binary indications of whether or not the corresponding risk factor is present for the corresponding patient. Presence of the corresponding identifier in risk factor subset 4025 denotes presence of the risk factor, and any other identifiers not included in risk factor subset 4025 are not present. For example, risk factor identifier 4026.A can indicate the first patient had significant exposure to asbestos, for example, based on extracting text from in textual data indicating that they currently work in a shipyard.

Other risk factors, such as risk factor C in this example, can optionally have additional information in patient history data, such as one or more quantitative values 4027. For example, risk factor C can correspond to a history of smoking, and the quantitative value 4027.C can indicate a number of years of smoking history and/or an average number of packs per day. In this example, the first and second patient both have a risk factor C with different quantitative values. For example, quantitative value 4027.C.1 can indicate the first patient smoked for 30 years and quantitative value 4027.C.2 can indicate the second patient smoked for 10 years. As another example, a risk factor corresponding to breast cancer family history can have multiple qualitative and/or quantitative values 4027 indicating each of a set of family members that had breast cancer, their relation to the patient, temporal data indicating when each of the set of family members had breast cancer, additional identifiers indicating treatments and/or recovery of each family member, and/or other relevant and/or structured information.

In some embodiments, some risk factors can correspond to abnormalities, such as abnormality data 442 identified by a human and/or automatically detected by an inference function that utilizes a computer vision model, that were identified in image past medical scans and/or a recently captured medical scan. For example, a particular risk factor included in a risk factor subset 4025 can identify presence of a particular abnormality classification category 444 and/or presence of a particular abnormality pattern category 445. As another example, a risk factor can identify presence of a particular abnormality classification category 444 and/or presence of a particular abnormality pattern category 445. As another example, a particular risk factor included in a risk factor subset 4025 can identify presence of lung nodules, where one or more corresponding quantitative value 4027 can denote a number of lung nodules and/or a measurement of each lung nodule. Utilizing abnormality data 442 to generate risk assessment scores 4006 is discussed in further detail in conjunction with FIGS. 12F and 12G.

In this example, the risk assessment score 4006 for each risk assessment category 4012 is generated by a corresponding risk assessment function 4005, where a set of risk assessment functions 4005.1-4005.R are performed upon patient history data 430.1 to generate a set of corresponding risk assessment scores 4006.1.1-4006.1.R for the first patient, and where the same set of risk assessment functions 4005.1-4005.R are performed upon patient history data 430.2 to generate a set of corresponding risk assessment scores 4006.2.1-4006.2.R for the second patient.

Different risk assessment functions 4005 can be a function of different risk factors, such as risk factors relevant to the particular disease and/or condition for the corresponding category. In this example, the first risk assessment function 4005.1 is a function of risk factors A, B, and C, while risk assessment function 4005. R is a function of risk factors A, D, and E. For example, risk factors A, B, and C are the only risk factors deemed relevant to the corresponding medical condition of risk factor category 4012.1, while risk factors A, D, and E are the only risk factors deemed relevant to the corresponding medical condition of risk factor category 4012.1. Some risk assessment functions 4006 can utilize every possible risk factor as input. Different risk assessment functions 4006 can utilize different sets of risk factors as input and/or different numbers of risk functions as input. As a particular example, one risk factor function corresponding to lung cancer can utilize a set of risk factors that include at least smoking history, exposure to asbestos, and/or detection of lung nodules in medical scans as input, while other risk factors such as allergy to peanuts or history of depression may not be utilized as input to the risk factor function corresponding to lung cancer.

In the particular example illustrated in FIG. 12B, the input to a given risk assessment function 4005 can include a set of binary identifiers indicating whether each relevant risk factor is present. The input to a given risk assessment function 4005 can optionally utilize the quantitative value 4027 of one or more relevant risk factors as input. For example, in computing risk assessment score 4006.1.1, a binary value of 1 is utilized for risk factor A based on risk factor A being present in risk factor subset 4025.1; a binary value of 0 is utilized for risk factor B based on risk factor B not being present in risk factor subset 4025.1; and the quantitative value 4027.C.1 is utilized for risk factor C based on being included in risk factor subset 4025.1. Similarly, in computing risk assessment score 4006.2.1 a binary value of 0 is utilized for risk factor A based on risk factor A not being present in risk factor subset 4025.2; a binary value of 1 is utilized for risk factor B based on risk factor B not being present in risk factor subset 4025.2; and the quantitative value 4027.C.2 is utilized for risk factor C based on being included in risk factor subset 4025.2.

As another example, in computing risk assessment score 4006.1.R, a binary value of 1 is utilized for risk factor A based on risk factor A being present in risk factor subset 4025.1; a binary value of 0 is utilized for risk factor D based on risk factor D not being present in risk factor subset 4025.1; and a binary value of 0 is utilized for risk factor E based on risk factor E not being present in risk factor subset 4025. Similarly, in computing risk assessment score 4006.2.R, a binary value of 0 is utilized for risk factor A based on risk factor A being absent from risk factor subset 4025.2; a binary value of 1 is utilized for risk factor D based on risk factor D not being present in risk factor subset 4025.2; and a binary value of 0 is utilized for risk factor E based on risk factor E not being present in risk factor subset 4025.2.

In some embodiments, one or more risk assessment functions can be expressed as a weighted sum of the binary and/or continuous values identified for each of the set of relevant risk factors in the risk factor subset. As a particular example, the risk assessment function 4005.1 can be expressed as α(A)+β(B)+γ(C), where α, β and γ are constant weights automatically determined by the risk assessment system and/or configured via user input, where A and B are binary values indicating whether or not risk factors A and B are present, respectively, and where C is the quantitative value 4027.C for risk factor C. The different weights can be configured based on the relative importance of each risk factor and/or based on normalizing the range of values that can populate any quantitative value 4027 supplied as input relative to the binary values. For example, the value of α can be greater than the value of β based on risk factor A being more likely to cause, more likely to be caused by, and/or being more highly correlated with the medical condition of risk factor category 4012.1 than risk factor B.

In some embodiments, the configuration of weights can be automatically performed by the risk assessment system and/or another subsystem 101 based on applying one or more analytic functions and/or machine learning models to historical data indicating a plurality of patient risk factors mapped to final diagnosis data for each of a plurality of patients. In other embodiments, the configuration of weights can be performed via user input by a medical professional and/or can be automatically set based on medical standards indicating relative importance of different risk factors.

Suppose this example risk factor function 4006.1 corresponds to risk of lung cancer. Risk factor identifier 4026.A can correspond to exposure to asbestos, and the risk factor identifier 4026.A can be present in the first patient's risk factor subset 40251.1 based on the first patient having had significant exposure to asbestos, for example, based on having worked in a shipyard. Risk factor identifier 4026.B can correspond to family history of lung cancer, where the risk factor identifier 4026.B is not present in the first patient's risk factor subset 40251.1 based on the first patient having no family history of lung cancer. Risk factor identifier 4026.A can correspond to exposure to smoking years, and quantitative value 4027.C.1 can be equal to 30 based on the first patient having a history of 30 years of smoking. The risk assessment score 4006.1.1 can be computed as α(1)+β(0)+γ(30). Note that in other embodiments, the risk factor function 4006 corresponding to risk of lung cancer can further be a function of: whether, how much, and/or how long a given patient was exposed to secondhand smoke; whether, how much, and/or how long a given patient had previous radiation therapy; whether, how much, and/or how long a given patient was exposed to radon gas; and/or whether, how much, and/or how long a given patient was exposed to one or more other carcinogens.

Other embodiments of risk assessment functions can optionally be other types of mathematical functions and/or inference functions of binary, discrete and/or continuous values for each of a subset of possible risk factors that are relevant to the corresponding risk factor category, where weights are optionally applied in a similar fashion to denote relative importance of different risk factor for different types of functions 4006.

FIG. 12C illustrates an example embodiment of the risk assessment database 4011 of FIG. 12A that includes a set of risk assessment categories 4012.1-4012.K that correspond to a set of diseases and/or other medical conditions 4013.1-4013.K. This set of risk assessment categories 4012.1-4012.K can correspond to the full set of risk assessment categories 4012.1-4012.R and/or can correspond to a proper subset of the full set of risk assessment categories 4012.1-4012.R.

In some embodiments, the risk assessment system 4002 is utilized prior to capturing of medical scans for a given patient. In such embodiments, the risk assessment system 4002 can be utilized to optimize the capturing of medical scans and/or the display of a resulting set of medical images based on one or more diseases and/or conditions that are identified as highest risk for the patient. In particular, the high risk protocol instructions 4015 for some or all risk assessment categories can include a contrast protocol 4021, an imaging protocol 4022, and/or a medical scan layout protocol 4023.

The contrast protocol 4021 can indicate types and/or dosages of contrasts to be administered to the patient for capturing of medical images and/or can indicate any other instructions regarding administering of contrasts to the patient. Risk assessment categories 4012 for different diseases can have same or different contrast protocols 4021. For example, the contrast protocol 4021 for a given risk assessment category is one of a plurality of possible contrast protocols 4021.

The imaging protocol 4022 can indicate scan type data 421, modality, anatomical region, and/or views of medical scans that should be captured, the type of imaging study that should be ordered, and/or can indicate any other instructions regarding the medical images to be captured for the patient. Risk assessment categories 4012 for different diseases can have same or different imaging protocols 4022. For example, the image protocol 4022 for a given risk assessment category is one of a plurality of possible image protocols 4022.

The medical scan layout protocol 4023 can indicate a hanging protocol and/or can otherwise indicate an arrangement and/or ordering of image data of the resulting medical scans when presented to a radiologist via an interactive interface 275 and/or client display device 270 of a client device 120. Risk assessment categories 4012 for different diseases can have same or different medical scan layout protocols 4023. For example, the medical scan layout protocol 4023 for a given risk assessment category is one of a plurality of possible medical scan layout protocols 4023.

For example, detection of abnormality patterns associated with a first disease can be optimized by applying a first contrast protocol, a first imaging protocol, and/or a first medical scan layout protocol, while detection of abnormality patterns associated with a second disease can be optimized by applying a second contrast protocol, a second imaging protocol, and/or a second medical scan layout protocol. If the first disease is determined to have a higher risk than the second disease for a given patient based on the corresponding risk factor scores and/or if the first disease is determined to be high risk for the given patient and the second disease is not determined to be high risk for the given patient, it can be ideal to capture medical scans by administering contrasts and/or by capturing types of images to optimize detection and/or characterization of the corresponding abnormality pattern indicative of the first disease.

As a particular example, if a given patient is identified as high risk for lung cancer based on 30 years of smoking and based on having worked in a shipyard, a contrast protocol 4021 and/or imaging protocol 4022 associated with the lung disease risk assessment category 4012, such as a contrast protocol 4021 and/or imaging protocol 4022 that optimize the detection and/or viewing of lung nodules, can be indicated in high risk protocol instructions 4015, which can be displayed to a technician or other medical professional responsible for administering medical scans of the patient. Once these medical scans are captured, they can be automatically displayed to a radiologist and/or other medical professional in accordance with the medical scan layout protocol 4023 of the high risk protocol instructions 4015, such as a hanging protocol associated with optimal detection and/or viewing of lung nodules.

FIG. 12D illustrates an example embodiment of the risk assessment database 4011 of FIG. 12A that includes a set of risk assessment categories 4012.K+1-4012.K+L that correspond to a set of one or more adverse contrast reaction categories 4023.1-4023.L. This set of risk assessment categories 4012.K+1-4012.K+L can correspond to the full set of risk assessment categories 4012.1-4012.R and/or can correspond to a proper subset of the full set of risk assessment categories 4012.1-4012.R. For example this set of risk assessment categories 4012.K+1-4012.K+L of FIG. 12D relating to adverse contrast reaction categories can be included in addition to the set of risk assessment categories 4012.1-4012.K relating to different diseases and/or medical conditions as illustrated in FIG. 12C.

The set of adverse contrast reaction categories 4023.1-4023.L can each correspond to a possible adverse reaction that a patient could have when contrasts are administered. For example, one or more of the set of adverse contrast reaction categories 4023.1-4023.L can correspond to allergic reactions to one or more types of contrasts, where the corresponding risk assessment scores 4006 denote the risk allergic reactions to these one or more types of contrasts. As another example, one or more of the set of adverse contrast reaction categories 4023.1-4023.L can correspond to anaphylactic shock, where the corresponding risk assessment scores 4006 denote the risk of anaphylactic shock in response to administering of contrast. In some cases, the corresponding risk assessment functions 4005 can be functions of: whether and/or how many times the patient had previously experienced anaphylactic shock; whether the patient has family members with history of anaphylactic shock; whether the patient had previously had allergic reactions to components included in one or more types of contrasts; whether the patient has allergies to other common triggers of anaphylaxis; and/or whether and/or how many times the patient had previously had any adverse reactions to contrast in previous medical scans.

In some cases, some or all of the set of risk assessment categories 4012.K+1-4012.K+L can have high risk protocol instructions indicating contrast protocols 4021. These contrast protocols 4021 can indicate that only a certain subset of contrasts be administered to the patient; can indicate that only up to a certain dosage of contrast be administered to the patient; can indicate one or more types of contrasts that should not be administered to the patient; can indicate that contrasts should be administered with extra caution and/or that the patient should be more closely monitored after administering of the contrasts; and/or can indicate that no contrast should be administered.

The example embodiment of FIG. 12D further illustrates that the risk assessment database 4011 of FIG. 12A can includes a set of risk assessment categories 4012.K+L+1-4012.K+L+M that correspond to a set of one or more imaging contraindication categories 4024.1-4024.M. One or more imaging contraindication categories 4024 can be included alternatively or in addition to the one or more adverse contrast reaction categories 4023. Some or all adverse contrast reaction categories 4023 alternatively be a subset of the set of imaging contraindication categories 4024.1-4024.M.

Some or all of the set of imaging contraindication categories 4024.1-4024.L can correspond to a type of contradicted metallic implant that could cause a patient to have an adverse reaction when undergoing one or more types of imaging studies. Some or all of the set of imaging contraindication categories 4024.1-4024.L can correspond to other types of contraindications for one or more imaging modalities. Each imaging contraindication category 4024 can have a corresponding imaging protocol 4022. Some or all of these imaging protocols 4022 can indicate a corresponding contraindication to performing an MRI and/or one or more other types of imaging studies upon one or more anatomical regions, can indicate that an MRI and/or one or more other types of imaging studies should not be performed due to the corresponding contraindication, and/or can indicate that imaging studies should be performed with caution due to the corresponding contraindication.

For example, one or more imaging contraindication categories 4024 can correspond to an absolute contraindicated metallic implant category and/or a surface foreign body category. The corresponding risk assessment scores 4006 can denote the risk of adverse reaction to imaging based on patient history data 430 indicating the patient has one or more known and/or suspected absolute contraindicated metallic implants and/or surface foreign bodies, for example, based on the patient history data 430 indicating one of more absolute contraindicated metallic implants were previously implanted in the patient and/or detected in the patient. For example, the risk assessment scores 4006 for one or more imaging contraindication categories 4024 can indicate high risk based on the patient history data 430 indicating the patient having an implantable pediatric sternum device, a metallic foreign body in eye, “Triggerfish” contact lens or other types of contact lens, gastric reflux device, insulin pumps, temporary transvenous pacing leads, and/or other contraindicated metallic implants and/or surface foreign bodies.

As another example, one or more imaging contraindication categories 4024 can correspond to a relatively contraindicated metallic implant category. The corresponding risk assessment scores 4006 can denote the risk of adverse reaction to imaging based on patient history data 430 indicating the patient has one or more known and/or suspected relatively contraindicated metallic implants, for example, based on the patient history data 430 indicating one of more relatively contraindicated metallic implants were previously implanted in the patient and/or detected in the patient. For example, the risk assessment scores 4006 for one or more imaging contraindication categories 4024 can indicate high risk based on the patient history data 430 indicating the patient having an implantable drug infusion pump, epidural catheter, feeding tubes, spinal fixation hardware, Halo, neurostimulation systems, bone fusion stimulator, Cochlear implants, intracranial vascular clips, EEG electrodes, ventricular catheters, breast tissue expanders, prosthetic heart valves, pacemakers, cardioverter defibrillators, pacing wires and loop recorders, penile implants, Foley catheter with temperature probe, other medical implants, shrapnel within their body, and/or other relatively contraindicated metallic implants. As another example, the risk assessment scores 4006 for one or more contraindicated metallic implant categories 4024 can indicate high risk based on the patient history data 430 indicating the patient is pregnant or is possibly pregnant.

The corresponding imaging protocols can indicate that: no MRI study should be administered; no MRI study should be administered for any clinically relevant anatomical region if an MRI contraindicated metallic foreign body is anywhere within the patient's body regardless of location; confirmation that no absolute and/or relative contraindicated metallic implants exist is required prior to capturing an MRI and/or other imaging study of the patient; any known absolute and/or relative contraindicated metallic implants must be removed prior to capturing an MM and/or other imaging study of the patient; and/or an MRI and/or other imaging studies be should be administered with caution due to the confirmed and/or suspected presence of an absolute contraindicated metallic implant, a relative contraindicated metallic implant, and/or other contraindication for MRI studies or other imaging studies.

As a particular example, a patient scheduled for an ankle MRI can be identified as high risk for an imaging contraindication category 4024 based on their patient history data 430 indicating a contraindicated eye contact lens may be present. The corresponding imaging protocol can require that, prior to the patient entering an MRI examination room for capturing of the ankle MRI, any contraindicated eye contact lens of the patient must first be removed and/or must otherwise be verified to be not present in the patient.

FIG. 12E illustrates an example embodiment of risk assessment system 4002 of FIG. 12A, where the high risk protocol instructions 4015.X are sent to a client device 120 for display via a client display device 270, for example, via interactive interface 275. In particular a contrast protocol 4021.X and/or an imaging protocol 4022.X for a particular risk assessment category 4012.X identified as high risk in the high risk category identification step 408 are sent to the client device for display to a technician and/or are otherwise communicated to the technician and/or another medical professional prior to capturing of medical scans of the patient, enabling the technician to facilitate capture of the medical scans of the patient accordingly.

This particular risk assessment category 4012.X can correspond to a risk assessment category associated with a disease and/or medical condition as discussed in conjunction with FIG. 12C. This particular risk assessment category 4012.X can correspond to a risk assessment category associated with an adverse reaction to contrast as discussed in conjunction with FIG. 12D, for example, where only a corresponding contrast protocol 4021.X is sent to the client device 120 for display.

This improves the technology of medical imaging by customizing the capturing of medical scans for different patients based on automatically identifying which types of conditions for which they have greater disposition based on risk factors of their patient history data, and by selecting one of a possible set of imaging protocols and/or one of a possible set of contrast protocols to optimize the detection of abnormalities associated with the corresponding condition in review of the resulting image data. This also improves the technology of patient care and contrast administration by automatically identifying patients at high risk for adverse reaction to contrasts to ensure that an appropriate contrast protocol is selected for these patients and/or to ensure that technicians are automatically alerted of this risk.

FIG. 12F illustrates an example embodiment of risk assessment system 4002 of FIG. 12A, for example, after one or more medical scans are captured for a patient. For example, the medical scans were captured in accordance with the contrast protocol 4021.X and/or an imaging protocol 4022.X identified as discussed in conjunction with FIG. 12E. As another example, the risk assessment system 4002 is performed upon the patient history data 430 for the first time for the given patient in conjunction with available medical scans that were captured for the patient.

In embodiments where the medical scans were captured in accordance with the contrast protocol 4021 and/or an imaging protocol 402 identified as discussed in conjunction with FIG. 12E, the image data 410 can be sent to the client device 120 for display via client display device 470 in accordance with the medical scan layout protocol 4023 indicated in the high risk protocol instructions 4015.X that are sent by the risk assessment system 4002 as discussed in conjunction with FIG. 12E. The medical scan layout protocol 4023 can optionally be sent to a client device configured to display the image data 410 to a radiologist that reviews the medical scans, where the contrast protocol and/or imaging protocol 4022 were sent to a different client device configured to display the image data 410 to a technician that is responsible for administering contrasts and/or capturing of the medical scans. This improves the technology of medical imaging by customizing the display of medical scans for different patients based on automatically identifying which types of conditions for which they have greater disposition based on risk factors of their patient history data, and by selecting one of a possible set of hanging protocols to optimize the detection of abnormalities associated with the corresponding condition in review of the resulting image data.

The image data 410 of one or more captured medical scans of the patient can alternatively or additionally be sent to risk assessment system 4002 from medical scan database 342, such as a medical picture archive system 2620 implementing the medical scan database 342. The risk assessment system 4002 can also receive patient history data 430 for the corresponding patient from the same or different location as the image data 410. The image data 410 can be processed via performance of one or more inference functions 4010 to generate abnormality data 442. For example, the one or more inference functions 4010 utilize a computer vision model trained upon a plurality of medical scan image data and corresponding labels. The inference functions 4010 can be operable to generate the abnormality data 442 as output that indicates detection or, characterization of, and/or measurements of one or more abnormalities in the image data of a medical scan. For example, the one or more inference functions 4010 are trained and/or performed by utilizing the medical scan image analysis system 112. The one or more inference functions 4010 can correspond to any inference function discussed herein and/or can be included in function database 346. The abnormality data 442 can indicate detection of, location of, and/or measurements of one or more abnormalities automatically detected in the image data 410 of the one or more medical scans.

The abnormality data 442 can alternatively be received via subsystem network interface 265, for example, from the medical scan database 342 and/or from another subsystem 101 that automatically generated the abnormality data 442 in inference data and/or diagnosis data 440. In some cases, the abnormality data 442 corresponds to and/or is based on human-generated abnormality data based on human annotations to image data of one or more medical scans of the patient and/or based on a medical report written by a human describing the image data of one or more medical scans of the patient.

Risk assessment functions 4035 that utilize abnormality data 442 as input alternatively or in addition to patient history data 430 can be performed to generate a set of risk assessment scores 4006.1-4006.R. The risk assessment functions 4035 can be the same as or similar to the risk assessment functions 4005 of FIGS. 12A-12E. For example, the same risk assessment functions 4005 can be applied regardless of whether abnormality data 442 is available as input. In other cases, the risk assessment functions 4035 are different from risk assessment functions 4005 because risk assessment functions 4035 utilize abnormality data 442 as input and risk assessment functions 4005 cannot handle abnormality data 442 as input. This can be ideal to differentiate the absence of detected abnormalities from the absence of abnormality data 442 due to medical scans not yet having been captured. In such cases, the risk assessment functions 4035 can be similar to risk assessment function 4005, where different weights are applied to risk factors of patient history data 430 based on also utilizing abnormality data 442 as input.

Some or all risk assessment scores 4006.1-4006.R of FIG. 12F can thus be a function of detected abnormalities in image data 410. In cases where risk assessment scores 4006.1-4006.R were previously generated for the patient prior to capturing of the medical scans as discussed in conjunction with FIG. 12E, the risk assessment scores 4006.1-4006.R can correspond to updated risk assessment scores 4006.1-4006.R from the previously generated risk assessment scores 4006.1-4006.R prior to the additional information supplied in abnormality data 442. For example, an updated risk assessment score 4006 for a given risk assessment category 4012 can have a value indicating a higher risk than the value of the previously generated risk assessment score 4006 for the given risk assessment category 4012 based on detection of abnormalities in the image data 410 that correspond to the disease and/or medical condition of the given risk assessment category 4012. As another example, an updated risk assessment score 4006 for a given risk assessment category 4012 can have a value indicating a lower risk than the value of increase from the previously generated risk assessment score 4006 for the given risk assessment category 4012 based on no detection of abnormalities in the image data 410 that correspond to the disease and/or medical condition of the given risk assessment category 4012.

For example, one or more risk assessment functions can utilize a binary identifier indicating whether or not an abnormality pattern and/or type of abnormality corresponding to and/or related to a disease and/or medical condition of the corresponding risk assessment category 4012 was detected and indicated in abnormality data 442. As another example, the one or more risk assessment functions can utilize one or more quantitative values as input indicating measurements of, a number of instances of, and/or severity of abnormality pattern and/or type of abnormality corresponding to and/or related to a disease and/or medical condition of the corresponding risk assessment category 4012 was detected and indicated in abnormality data 442.

In some cases, performing the one or more risk assessment functions includes generating a severity score and/or time-sensitivity score for detected abnormalities in abnormality data 442. For example, larger lesions and/or nodules detected in first image data are assigned higher severity scores and/or time-sensitivity scores than smaller lesions and/or nodules detected in second image data. As another example, a first abnormality pattern detected in first image data that corresponds to a first disease is assigned a higher severity score and/or time-sensitivity score than a second abnormality pattern detected in second image data that corresponds to a second disease that is less severe and/or time-sensitive than the first disease. As another example, a first abnormality detected to have grown in size by a first amount and/or at a first rate based on registration of the first abnormality in another, previous medical scan captured of a same first patient is assigned higher severity scores and/or time-sensitivity scores than a second abnormality detected to have grown in size by a second amount and/or at a second rate based on registration of the second abnormality in another, previous medical scan captured of a same second patient based on the first amount and/or first rate being greater than the second amount and/or the second rate.

In some cases, the severity score and/or time-sensitivity score is generated by performing the one or more inference functions 4010 and/or is included in the abnormality data 442. A risk assessment score 4006 for a first patient can indicate a first value while a risk assessment score 4006 for a first patient can indicate a second value. The first risk can indicate a higher risk than the second value based on the abnormality data 442 generated for the image data 410 of the first patient indicating a first severity score and/or first time-sensitivity score for its one or more detected abnormalities that higher than and/or otherwise indicates greater severity and/or time-sensitivity than a second severity score and/or second time-sensitivity score of the abnormality data 442 generated for the image data 410 of the second patient.

As a particular example, the example risk factor function 4006.1 corresponding to risk of lung cancer as discussed in conjunction with FIG. 12B further utilizes a risk factor as input denoting whether or not any lung nodules were detected in medical scans of the corresponding patient. As another particular example, the example risk factor function 4006.1 corresponding to risk of lung cancer as discussed in conjunction with FIG. 12B further utilizes a risk factor as input denoting how many lung nodules were detected in medical scans of the corresponding patient and/or denoting sizes of lung nodules that were detected in medical scans of the corresponding patient. For example, a risk assessment score for risk of lung cancer generated for a first patient with a history of smoking and exposure to asbestos with no detected lung nodules in image data 410 of their medical scans has a first value indicating a lower risk of lung cancer than a second value generated for a second patient with a history of smoking, and exposure to asbestos, and detected lung nodules in image data 410 of their medical scans. As another example, a risk assessment score for risk of lung cancer generated for a first patient, prior to capturing of medical scans, with a history of smoking and exposure to asbestos of their medical scans has a first value indicating a lower risk of lung cancer than a second value generated for the first patient, after capturing of medical scans with detected lung nodules in image data 410.

The high risk category identification step 4008 can be performed as discussed in conjunction with FIG. 12A. The high risk protocol instructions 4015 for one or more risk assessment categories identified as high risk can similarly be executed and/or communicated to a medical professional. As medical scans were already captured for the patient, the risk assessment system 4002 optionally evaluates risk assessment categories relating to diseases and/or medical conditions, as contrast was already administered, and adverse contract reaction is not of concern.

In embodiments where the high risk category identification step 4008 was previously performed based on previously generated risk assessment scores 4006.1-4006.R without utilizing image data 410, the high risk category identification step 4008 can again be performed utilizing the updated risk assessment scores 4006.1-4006.R. As these updated risk assessment scores 4006.1-4006.R may be different from the previously generated risk assessment scores 4006.1-4006.R based on the utilization of abnormality data 442 in performing the risk assessment functions 4005, a different one or more high risk categories may be identified. As illustrated in FIG. 12F, new high risk protocol instructions 4015.Y corresponding to a risk assessment category 4012.Y that is different from a risk assessment category 4012.X and is identified as high risk based on the updated risk assessment score 4006.Y is identified and communicated to client device 120. The abnormality data 442 and/or the image data 410 of the medical scan can also be communicated to client device 120, for example, for display via interactive interface 275.

For example, updated risk assessment score 4006.Y compares favorably to high risk score threshold 4014.Y and previously generated risk assessment score 4006.Y did not compare favorably to high risk score threshold 4014.Y based on detection of abnormalities in image data 410 that correspond to a disease and/or medical condition of risk assessment category 4012 and/or based on the patient history data 430 not including other risk factors sufficient in identifying the corresponding disease and/or medical condition as high risk.

As another example, updated risk assessment score 4006.X compares unfavorably to high risk score threshold 4014.X despite previously generated risk assessment score 4006.X comparing favorably to high risk score threshold 4014.X as discussed in conjunction with FIG. 12E, based on the absence of abnormalities in image data 410 that correspond to a disease and/or medical condition of risk assessment category 4012, despite patient history data 430 including other risk factors sufficient in identifying the corresponding disease and/or medical condition as high risk prior to capturing of the medical scan.

In some cases, the abnormality data 442 is sent to medical scan database 342 for storage, for example, in conjunction with the medical scan's diagnosis data 440. In such embodiments the abnormality data 442 is optionally included in patient history data 430 for subsequent uses of risk assessment system 4002 for the same patient, for example, prior to capture of subsequent medical scans.

Such an embodiment is illustrated in FIG. 12G, which illustrates an example embodiment of the risk assessment system 4002 of FIG. 12A. The patient history data 430 can include abnormality data 442, for example, as longitudinal data 433, which can be utilized to identify additional risk factors based on abnormalities detected in and/or absent from image data of previously captured medical scans. This abnormality data 442 may have been previously generated by risk assessment system 4002 as discussed in conjunction with FIG. 12F. Alternatively, the abnormality data 442 may have been previously generated by another subsystem 101 via performance of one or more inference functions, based on user input, and/or based on human review of corresponding medical scans. The risk assessment category 4012.X is identified based on applying some or all risk assessment functions 4005 to abnormality data 442 of the patient history data 430. For example, the one or more risk assessment functions 4005 are implemented as risk assessment function 4035 to handle abnormality data 4035 as input. The high risk protocol instructions 4015.X are executed and/or sent to the client device 120 for display as discussed previously.

As a particular example, fat and/or muscle composition can be detected and/or measured by one or more inference functions 4010 in medical scans captured for patients and can be included in abnormality data 442. This fat and/or muscle composition can be used to determine additional patient risk factors of patient history data as necessary for future medical scans and/or patient visits. For example, if one or more medical scans are captured for the patient due to symptoms and/or suspicion of appendicitis, the fat and/or muscle composition can be measured in image data 410 for scans of the entire abdomen captured to characterize the appendicitis, despite this information not necessarily being relevant to the suspicion of appendicitis. While this and/or muscle composition information may not be pertinent at this time, it can be stored in patient history data 130 of the patient. This fat and/or muscle composition can then be utilized as risk factors for other conditions such as stroke and/or joint disorders at that time and/or at a future date. For example, the patient may have other symptoms and/or risk factors indicative of stroke and/or joint disorders at a future date, and the fat and/or muscle composition from the prior appendicitis scan included in the patient history 130 can be utilized as input to risk assessment functions 4005 for a risk assessment category associated with risk of stroke and/or for one or more risk assessment categories associated with risk of one or more joint disorders to generate corresponding risk assessment scores indicating risk of stroke and/or joint disorders. Other types of measurements and/or characterizations of anatomical features present in medical scan captured for patients can similarly be automatically collected by default, regardless of the condition for which the scan was captured, and these measurements and/or characterizations of anatomical features can be included in patient history data 130 of the patient for use in generating risk assessment scores 4006 via risk assessment functions 4005 at that time or in the future.

In some embodiments, one or more inference functions 4010 can be performed in accordance with a radiomics method and/or one or more data-characterization algorithms. One or more inference functions 4010 can be applied to the medical image 410 generate one or more radiomic features as abnormality data 442, where the or one or more inference functions 4010 are optionally implemented utilizing a radiomic method and/or one or more data-characterization algorithms. In some embodiments, any of the abnormality data 442 and/or diagnosis data 440 discussed herein can include and/or be based on one or more radiomic features extracted by applying an radiomic method and/or one or more data-characterization algorithms. As a particular example, a radiomic method and/or one or more data-characterization algorithms can be utilized to extract radiomic features indicating and/or relating to fat and/or muscle composition extracted from image data 410 of a medical scan, where these radiomic features indicating and/or relating to fat and/or muscle composition are utilized to update patient history data 430 and/or to determine additional risk factor data for the patient as discussed above.

FIG. 12H illustrates an example of a risk assessment database 4011 of FIG. 12A that includes high risk protocol instructions indicating triage prioritization data 4032 and/or triage instruction data 4033 for diseases and/or medical condition of corresponding risk assessment categories. The triage prioritization data 4032 can correspond to a fixed triage priority score for the corresponding risk assessment category and/or can include function that is utilized to calculate the triage priority score as a function of the corresponding risk assessment score for a given patient. The triage instruction data 4033 can denote other information denoting how the patient can be triaged.

In particular, different diseases may have triage prioritization data indicating how patients be prioritized for capturing of medical scans, how medical scans for different patients be prioritized for review, and/or how patients be prioritized for treatment. For example, patients at high risk for a first disease that is more severe and/or more time sensitive than a second disease can be prioritized higher for triage than patients at high risk for the second disease to ensure patients at risk for more severe and/or more time sensitive diseases are prioritized. As another example, patients at high risk for a first disease can be triaged for treatment and/or for their medical scans to be reviewed by a first type of specialist associated with expertise in the first disease, while patients at high risk for a second disease can be triaged for treatment and/or for their medical scans to be reviewed by a second type of specialist associated with expertise in the second disease.

Alternatively or in addition, patients with different levels of risk for a given medical condition, given their patient history data, can be prioritized according to their respective levels of risk. For example, a first patient with a first risk assessment score 4006 for a given risk assessment category 4012 associated with a given disease can be prioritized higher than a second patient with a second risk assessment score 4006 for the given risk assessment category 4012 associated with the given disease based on the first risk assessment score 4006 indicating higher risk than the second risk assessment score.

FIGS. 12I and 12J illustrate example embodiments of the risk assessment system 4002 of FIG. 12A that utilize the triage prioritization data 4032 of FIG. 12G to generate triage prioritization scores 4034 for each of a plurality of patients 1-P. Each triage prioritization score 4034 can be identified and/or calculated based on the triage prioritization data 4032 for one or more risk assessment categories 4012 identified as high risk. The different patients can have different diseases of different risk assessment categories identified as high risk based on the risk assessment scores 4006 calculated as a function of their respective patient history data 430 and/or abnormalities detected in image data 410 of their medical scans, which can induce different triage prioritization scores be generated for the different patients. In these example, patient 1 is assigned a triage prioritization score 4034.X which can be a same or different value than a triage prioritization score 4034.Y assigned to patient P. This improves the technology of medical triage by automatically prioritizing triage based on risk factors for different patients that may affect the likelihood that certain patients have serious medical conditions requiring urgent review and/or care.

Each triage prioritization score 4034 can be a function of severity of the corresponding disease and/or a function of time-sensitivity of the corresponding disease. For example, the triage prioritization data 4032 of a given risk assessment category 4012 can indicate a constant value to be assigned to as triage prioritization score 4034 for patients identified as high risk for the given risk assessment category 4012, where the constant value of more time-sensitive and/or severe diseases indicates a higher priority than the constant value of less time-sensitive and/or severe diseases.

The triage prioritization score 4034 can optionally be a function of the patient's risk assessment score for the corresponding category. For example, the triage prioritization data 4032 of a given risk assessment category 4012 can indicate a function for calculating the triage prioritization score as a function of the constant value assigned to the given risk assessment category 4012 and the patient's risk factor score 4006 for the given risk assessment category 4012, and can include computing a sum and/or product of the patient's risk assessment score 4006 and the constant value, where the constant value utilized in functions for risk assessment categories 4012 corresponding to more severe and/or time sensitive diseases is greater than and/or induces higher priority scores than the constant value utilized in functions of risk assessment category 4012 corresponding to less time-sensitive and/or severe diseases.

In some embodiments, the triage prioritization score 4034 is only generated for a patient based on triage prioritization data 4032 of a corresponding risk assessment category 4012 when the patient's risk assessment score 4006 for the corresponding risk assessment category 4012 compares favorably to the high risk score threshold 4014 for the corresponding risk assessment category 4012 and/or otherwise deems the patient as high risk for the corresponding risk assessment category 4012.

In some embodiments, if a patient has no risk assessment scores 4006 indicating corresponding risk assessment categories 4012 are high risk for the patient, the triage prioritization score 4034 is automatically set to a default triage prioritization score for the patient that is a least favorable score and/or is less favorable than triage prioritization score for all other patients with at least one risk assessment score 4006 indicating high risk.

In some embodiments, a patient is identified as high risk for multiple risk assessment categories, for example, based on multiple risk assessment scores 4006 each comparing favorably to the high risk score threshold 4014 of the corresponding risk assessment category and/or otherwise indicating high risk for multiple risk assessment categories 4012. In such cases, the triage prioritization score 4034 can be calculated based on utilizing the triage prioritization data 4032 of each category in which the patient is identified as high risk. For example, the triage prioritization score 4034 can correspond to a function, such as a sum and/or a maximum, of the prioritization scores calculated individually for each of these high risk categories in accordance with their triage prioritization data 4032.

For example, the triage prioritization score 4034 for a first patient has a first value based on a risk assessment score 4006 for the first patient indicating high risk for a risk assessment category 4012 corresponding to a first disease, and the triage prioritization score 4034 for a second patient has a second value based on a risk assessment scores 4006 for the second patient indicating high risk for a risk assessment category 4012 corresponding to a second disease.

In some embodiments, the first value can indicate a more favorable priority than the second value based on the first disease being more severe and/or time sensitive than the second disease. Alternatively, the first value can indicate a less favorable priority than the second value despite the first disease being more severe and/or time sensitive than the second disease based on the first patient's risk factor score for the first disease indicating a lower risk than the second patient's risk factor score for the second disease.

The first value can indicate a more favorable priority than the second value based on the first patient's risk factor score for the first disease indicating a higher risk than the second patient's risk factor score for the second disease. Alternatively, the first value can indicate a less favorable priority than the second value despite the first patient's risk factor score for the first disease indicating a higher risk than the second patient's risk factor score for the second disease based on the second disease being more severe and/or time sensitive than the first disease.

As a particular example, a first patient has patient history data 430 indicating history of smoking and significant exposure to asbestos. In performing inference function 4010, no lung nodules are detected in the image data 410 of their medical scans. A risk assessment score 4006 for a lung cancer risk assessment category 4012 calculated for the first patient via a risk assessment function 4005 has a first value that compares favorably to the high risk score threshold 4014 based on the first patient's history of smoking and significant exposure to asbestos, despite the lack of detection of lung nodules. Meanwhile, a second patient has patient history data 430 also indicating history of smoking and significant exposure to asbestos. In performing inference function 4010, lung nodules are also detected in the image data 410 of their medical scans. A risk assessment score 4006 for a lung cancer risk assessment category 4012 calculated for the second patient via the risk assessment function 4005 has a second value that also compares favorably to the high risk score threshold 4014 based on the first patient's history of smoking, significant exposure to asbestos, and presence of lung nodules in their medical scan.

Both patients have triage prioritization scores 4034 generated based on the triage prioritization data 4032 of the lung cancer risk assessment category 4012 based on both being identified as high risk. The triage prioritization data 4032 of the lung cancer risk assessment category 4012 can indicate the triage prioritization score 4034 be calculated as an increasing function of the risk assessment score 4006 for the lung cancer risk assessment category 4012, and optionally as a function of a constant value assigned to the lung cancer risk assessment category 4012 based on the severity and/or time-sensitivity of lung cancer relative to diseases of other categories. Because the second value is higher than the first value based on the second patient having the detected of lung nodules in their medical scan and based on the first patient not having detected lung nodules in their medical scan, the triage prioritization score 4034 generated for the second patient has a value indicating a higher priority than the value of the triage prioritization score 4034 generated for the first patient. The second patient is prioritized more highly for medical care than the first patient and/or their medical scans are prioritized for human review by a radiologist more highly than the medical scans of the first patient.

As illustrated in FIG. 12I, the triage prioritization scores 4034 can indicate an ordering of the plurality of patients for triage by a triaging system 4040, for example, denoting an ordering in which the patients should be prioritized for capturing of medical scans, for treatments, and/or for other appointments with medical professionals. Patients with higher priority triage prioritization scores 4034 are prioritized higher for triage than patients with lower priority triage prioritization scores 4034. In this example, patient 2 has a highest priority triage prioritization score 4034, and is triaged via a triaging system 4040 with a highest priority, while patient P has a lowest priority triage prioritization score 4034, and is triaged via a triaging system 4040 with a lowest priority. For example, the triaging system 4040 maintains an ordering of patients in accordance with their triage prioritization score denoting the ordering in which the patients should be seen for medical care by a medical professional. In some cases, the patients are triaged by triaging system 4040 for capturing of medical scans, and the medical scans are captured for each patient utilizing the contrast protocol 4021 and/or the imaging protocol 4022 based on a risk assessment category identified as high risk for each patient as discussed in conjunction with FIG. 12E.

As illustrated in FIG. 12I, the triage prioritization scores 4034 can alternatively or additionally indicate an ordering that a plurality of medical scans captured for the plurality of patients should be triaged for review by a medical professional via the same or different triaging system 4040, for example, denoting an ordering in which captured medical scans of different patients should be prioritized for review. In this example, as the medical scans have already been captured, the medical scans are also processed via inference functions 4010 of FIG. 12F, and resulting abnormality data is utilized as input to the risk assessment functions 4035 as discussed in conjunction with FIG. 12F to generate the risk assessment scores for each patient. Patients with higher priority triage prioritization scores 4034 have their medical scans prioritized higher for review than the medical scans of patients with lower priority triage prioritization scores 4034. In this example, patient 3 has a highest priority triage prioritization score 4034, and their medical scan with image data 3 is triaged via a triaging system 4040 with a highest priority, while patient P has a lowest priority triage prioritization score 4034, and their medical scan with image data P is triaged via a triaging system 4040 via a triaging system 4040 with a lowest priority. The triage prioritization score can optionally implement the scan priority data 427 of FIG. 4A mapped to medical scans of the corresponding patient.

In some embodiments, a set of patients are first triaged via triaging system 4040 as discussed in conjunction with FIG. 12I prior to capturing of medical scans, and the resulting medical scans captured of some or all of the set of patients are then triaged for review as illustrated in FIG. 12J. In such embodiments, the triage prioritization score for some patients may change based on utilizing the abnormality data detected in medical scans of the patients, and the resulting ordering of the patients by their image data in triaging system 4040 of FIG. 12J can therefore be different from the prior ordering of the patients by triaging system 4040 of FIG. 12I. For example, patient 2 is no longer the highest priority patient based on their image data not including any detected abnormalities, while patient 3 has a highest priority after processing of their image data based on their image data including detected abnormalities.

In various embodiments, a risk assessment system includes a network interface; a processing system that includes a processor, and/or a memory device that stores executable instructions. The executable instructions, when executed by the risk assessment system, configure the processor to perform operations that include receiving, via the network interface, patient history data for a patient; generating a set of risk assessment scores corresponding to the patient for a set of risk assessment categories based on applying at least one risk assessment function to the patient history data; identifying one of the set of risk assessment categories as high risk for the patient based on a corresponding one of the set of risk assessment scores; identifying a high risk protocol corresponding to the one of the set of risk assessment categories; and/or facilitating performance of the high risk protocol for the patient based on identification of the one of the set of risk assessment categories as high risk for the patient, for example, by transmitting, displaying, and./or executing high risk protocol instructions of the high risk protocol.

FIG. 12K presents a flowchart illustrating a method for execution by a risk assessment system 4002 and/or other subsystem 101 that stores executional instructions that, when executed by at least one processor, cause the system to perform the steps below.

Step 4082 includes receiving, patient history data for a patient, for example, via a network interface. Step 4084 includes generating a set of risk assessment scores corresponding to the patient for a set of risk assessment categories based on applying at least one risk assessment function to the patient history data. Step 4086 includes identifying one or more of the set of risk assessment categories as high risk for the patient based on one or more of the set of risk assessment scores. Step 4088 includes identifying a high risk protocol corresponding to the one or more of the set of risk assessment categories. In various embodiments, the set of risk assessment categories include a set of disease-based risk assessment categories for each of a set of diseases, where the one of the set of risk assessment categories corresponds to one of the set of diseases.

Step 4090 includes facilitating performance of the high risk protocol for the patient based on identification of the one of the set of risk assessment categories as high risk for the patient. For example, high risk protocol instructions corresponding to the high risk protocol are transmitted to a client device for display and/or execution, and/or are executed by the risk assessment system itself. In cases where none of the set of risk assessment categories are identified as high risk for the patient, no high risk protocol is initiated for the patient.

In various embodiments, the method includes comparing each of the set of risk assessment scores to a risk assessment score threshold, for example, of a corresponding one of the set of risk assessment categories. The risk assessment score threshold can be the same or different for different risk assessment categories. The one of the set of risk assessment categories is identified as high risk based on having a corresponding one of the set of risk assessment scores that compares favorably to a risk assessment score threshold, for example, based on indicating a risk greater than or equal to a risk of the risk assessment score threshold.

In various embodiments, the method includes identifying an ordering of the set of risk assessment categories for the patient based on the set of risk assessment scores. The one of the set of risk assessment categories is identified as high risk based on being a most-favorably ordered risk assessment category in the ordering of the set of risk assessment categories. Alternatively or in addition, the one of the set of risk assessment categories is identified as high risk and another one of the set of risk assessment categories is not identified as high risk based on the risk assessment score of the one of the set of risk assessment categories being more favorable than the risk assessment score of the another one of the set of risk assessment categories.

In various embodiments, the method includes receiving, via the network interface, at least one medical scan of the patient. The method further includes generating abnormality data for the medical scan by performing at least one inference function that utilizes a computer vision model upon image data of the medical scan, where the computer vision model is trained on a plurality of training medical images. Some or all of the set of risk assessment scores corresponding to the patient for the set of disease-based risk assessment categories set is based on applying the at least one risk assessment function to the patient history data and to the abnormality data. In various embodiments, the high risk protocol corresponding to the one of the set of risk assessment categories includes one of a set of possible medical scan image layout protocols based on the one of the set of diseases, and facilitating performance of the high risk protocol for the patient includes facilitating display of the at least one medical scan in accordance with the one of the set of possible medical scan image layout protocols via display device of a client device.

In various embodiments, facilitating performance of the high risk protocol includes generating triage data for the patient based on the one of the set of diseases and further includes automatically triaging the patient based on the triage data. In various embodiments, the method includes receiving, via the network interface, patient history data for a plurality of patients that includes the patient. A plurality of sets of risk assessment scores corresponding to the plurality of patients are generated for the set of disease-based risk assessment categories based on applying the at least one risk assessment function to the patient history data of each of the plurality of patients. A plurality of triage data is generated for the plurality of patients based on the plurality of sets of risk assessment scores.

In various embodiments, the triage data for the patient indicates a first priority. A second patient has triage data in the plurality of triage data indicating a second priority, and the first priority is more favorable than the second priority based on the one of the set of risk assessment scores of the patient for the one of the set of risk assessment categories corresponding to the one of the set of diseases indicating a higher risk than the one of the set of risk assessment scores of the second patient for the one of the set of risk assessment categories corresponding to the one of the set of diseases.

In various embodiments, another one of the set of disease-based risk assessment categories corresponding to another one of the set of diseases is identified high risk for a second patient based on one of the plurality of sets of risk assessment scores generated for the second patient. The triage data of the patient indicates a first priority, and the second patient has triage data in the plurality of triage data indicating a second priority. The first priority is more favorable than the second priority based on the one of the set of diseases being one of: more severe than the another one of the set of diseases, or more time-sensitive than the another one of the set of diseases.

In various embodiments, the high risk protocol includes one of a set of possible image protocols based on the one of the set of diseases. Facilitating performance of the high risk protocol further includes generating a notification for display via a display device of at least one client device recommending capturing of at least one medical scan of the patient via the one of the set of possible image protocols. In various embodiments, the high risk protocol alternatively or additionally includes one of a set of possible contrast protocols corresponding to the one of the set of diseases. Facilitating performance of the high risk protocol alternatively or additionally includes generating a notification for display via a display device of at least one client device recommending administering contrast to the patient for capturing of the at least one medical scan in accordance with the one of the set of possible contrast protocols.

In various embodiments, the method further includes receiving, via the network interface, at least one medical scan of the patient, where the least one medical scan is captured based on the one of the set of possible image protocols and/or the one of the set of possible contrast protocols indicated in display of the high risk protocol. Abnormality data is generated for the at least one medical scan by performing at least one inference function that utilizes a computer vision model, where the computer vision model is trained on a plurality of training medical images. The method includes generating an updated risk assessment score for the one of the set of risk assessment categories based on the abnormality data and based on the patient history data.

In various embodiments, the updated risk assessment score for the one of the set of risk assessment categories indicates a higher risk than the risk assessment score for the one of the set of risk assessment categories based on the abnormality data indicating at least one detected abnormality pattern that corresponds to the one of the set of diseases. In other embodiments, the updated risk assessment score for the one of the set of risk assessment categories indicates a lower risk than the risk assessment score for the one of the set of risk assessment categories based on the abnormality data indicating no detected abnormality pattern that corresponds to the one of the set of diseases.

In various embodiments, the method includes generating updated patient history data for the patient based on the abnormality data. Storage of the updated patient history data is facilitated, for example, in a database and/or mapped to a patient identifier of the patient. The method can further include retrieving the updated patient history data from storage, for example, at a later time and/or data associated with a new appointment with the patient and/or a new medical concern associated with the patient. An updated set of risk assessment scores corresponding to the patient for a set of risk assessment categories can be generated based on applying the at least one risk assessment function to the updated patient history data, for example, where the updated set of risk assessment scores are generated as a function of the abnormality data included in the updated patient history data.

In various embodiments, the abnormality data indicates fat composition data of anatomical features included in the at least one medical scan and/or muscle composition data of anatomical features included in the at least one medical scan. Another one of the set of risk assessment categories corresponds to one of: a stroke category or a joint disorder category. One of the updated set of risk assessment scores corresponding to the another one of the set of risk assessment categories is generated based on the fat composition data and/or or the muscle composition data. The method further includes identifying the another one of the set of risk assessment categories as high risk for the patient based on the one of the updated set of risk assessment scores.

In various embodiments, the one of the set of risk assessment categories corresponds to an adverse contrast reaction category for at least one of a set of possible contrast protocols. The high risk protocol corresponds to a contrast administering protocol for medical imaging of the patient that indicates not administering contrast in accordance with the at least one of the set of possible contrast protocols. In various embodiments, facilitating performance of the contrast administering protocol includes generating a notification for display via a display device of at least one client device recommending not administering contrast in accordance with the at least one of the set of possible contrast protocols. In various embodiments, the adverse contrast reaction category corresponds an anaphylactic shock category and/or an allergic reaction category.

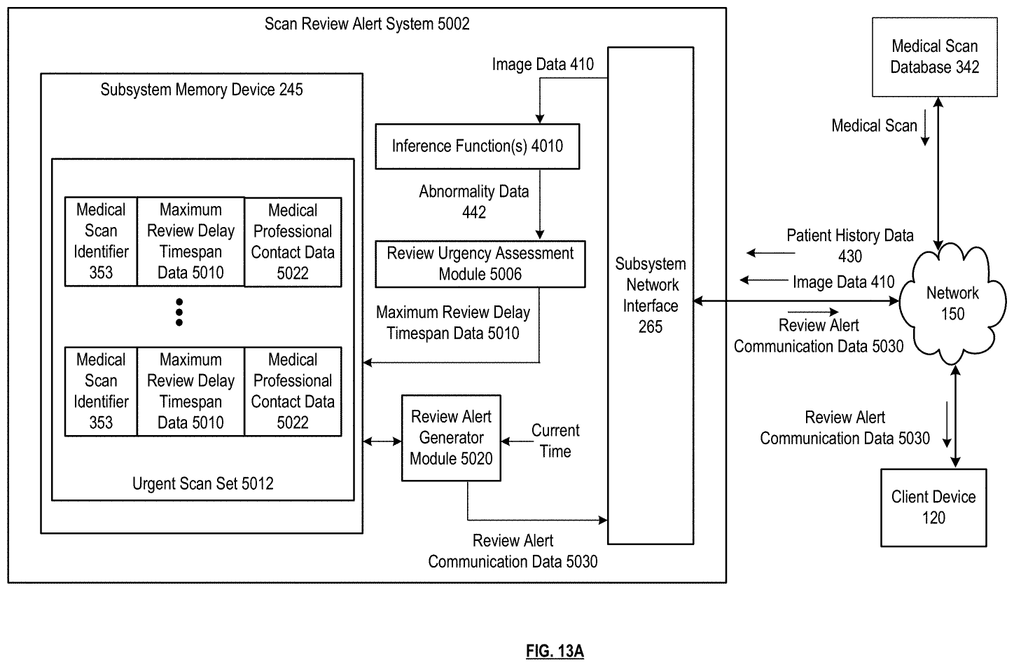

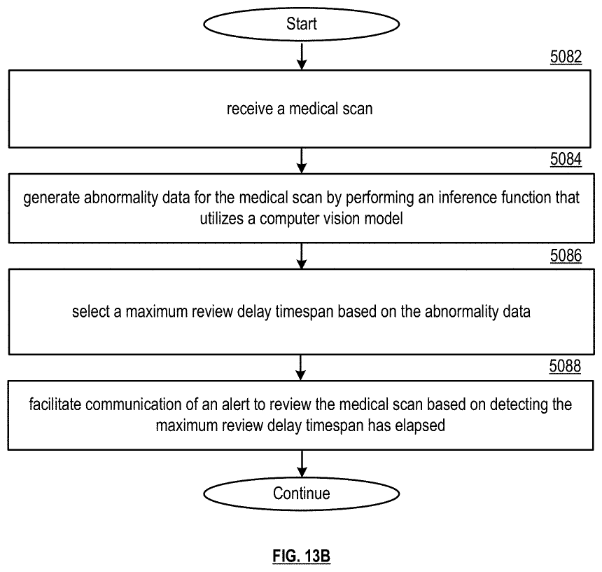

FIG. 13A presents an embodiment of a scan review alert system 5002. Patients may have critical conditions indicated in their medical scan that go undetected until the scan is reviewed by a radiologist. If this condition was never suspected by the referring clinician, the scan may be low priority and remain unreviewed for a long time. To mitigate this problem, if an urgent condition is detected automatically by utilizing a computer vision model trained to detect, characterize, and/or measure abnormalities in medical scans by applying one or more corresponding inference functions as discussed previously, escalation can be initiated immediately and/or after a predetermined amount of time has elapsed via communicating an alert to one or more designated points of contact. In particular, if urgent condition is detected in output of an inference function performed upon a medical scans captured for a particular patient, review of the medical scan and/or care of the patient can be automatically escalated by alerting the detection of the urgent condition to one or more identified contacts associated with the patient, such as the referring physician, a radiologist assigned to review the medical scan, one or more other radiologists available to review the scan more promptly than the assigned radiologist, one or more family members and/or emergency contacts of the patient, and/or the patient themselves.

The scan review alert system 5002 can communicate bi-directionally, via network 150, with the medical scan database 342 and/or other databases of the database storage system 140, with one or more client devices 120, and/or, while not shown in FIG. 13A, one or more subsystems 101 of FIG. 1. In some embodiments, the scan review alert system 5002 is an additional subsystem 101 of the medical scan processing system 100, implemented by utilizing the subsystem memory device 245, subsystem processing device 235, and/or subsystem network interface 265 of FIG. 2B. In some embodiments, some or all of the scan review alert system 5002 is implemented by utilizing other subsystems 101 and/or is operable to perform functions or other operations described in conjunction with one or more other subsystems 101.

The scan review alert system 5002 can be operable to perform one or more inference functions upon image data of one or more medical scans captured for a patient, for example, immediately after being captured and/or prior to their review by a radiologist or other medical professional. The inference functions 4010 can be the same or different from inference function 4010 of FIG. 12F. The inference functions 4010 can utilized a computer vision model trained utilizing a training set of medical scans and corresponding labeling data. The inference functions 4010 can be operable to generate the abnormality data 442 as output that indicates detection or, characterization of, and/or measurements of one or more abnormalities in the image data of a medical scan. For example, the one or more inference functions 4010 are trained and/or performed by utilizing the medical scan image analysis system 112. The one or more inference functions 4010 can correspond to any inference function discussed herein and/or can be included in function database 346.

The scan review alert system 5002 can identify any time-sensitive and/or urgent conditions evident in abnormality data 442 generated for given image data 410 by utilizing a review urgency assessment module 5006. This can optionally include applying one or more risk assessment functions 4035 to the abnormality data and/or patient history data 430 to generate risk assessment scores 4006 for one or more risk factor categories. This can optionally include generating a severity score and/or time-sensitivity score for one or more abnormalities automatically detected in the image data 410 of the one or more medical scans based on measurements and/or characterization of these abnormalities as indicated in abnormality data 442, for example, as discussed in conjunction with FIG. 12F.

The review urgency assessment module 5006 can generate maximum review delay timespan data 5010 based on the abnormality data 442. For example, the maximum review delay timespan data 5010 can indicate an amount of time, a deadline time and/or date, and/or a window of time in which the medical scan should be reviewed by a medical professional. In particular, the maximum review delay timespan data 5010 can correspond to a “countdown timer” set as a function of the urgency of the condition and/or as a function of the patient's risk factors, where an alert is transmitted to an assigned radiologist contacted once this countdown timer has elapsed. In some embodiments, if the assigned radiologist is not available and/or cannot be reached when this countdown timer has elapsed, other relevant contacts included in a list of contacts can be alerted to ensure the medical scan is reviewed. In some embodiments, to ensure that not too many personnel are alerted unnecessarily, the list is ordered and a given contact is only alerted when one or more prior contacts in the list are unavailable and/or cannot be reached, where alerts are transmitted successively down the list until at least one contact is successfully reached.

The maximum review delay timespan data 5010 can be generated as a function of a severity score and/or time-sensitivity score indicated in abnormality data 442 and/or generated by the review urgency assessment module 5006 based on the abnormality data 442. In particular, the maximum review delay timespan data 5010 can indicate larger time windows for medical scans that are normal and/or include less severe and/or time-sensitive types of abnormalities detected in abnormality data 442, as the review of these medical scans is not particularly urgent. The maximum review delay timespan data 5010 can indicate smaller time windows for medical scans that include more severe and/or time-sensitive types of abnormalities detected in abnormality data 442, as the review of these medical scans may be more urgent due to the detection of a more severe and/or time-sensitive condition and/or abnormality.

In some cases, the scan review alert system 5002 can implement and/or communicate with the risk assessment system 4002. Risk assessment scores 4006 can be generated based on abnormality data 442 and/or patient risk factors 430 identified for the patient. The maximum review delay timespan data 5010 can be generated as a function of the risk assessment scores 4006 generated via applying one or more risk assessment function 4035. For example, the maximum review delay timespan data 5010 can indicate larger time windows when none of the risk assessment scores 4006 indicate high risk, as the review of these medical scans is not particularly urgent. The maximum review delay timespan data 5010 can indicate smaller time windows when one or more risk assessment scores 4006 indicate high risk in a corresponding category and/or when one or more risk assessment scores 4006 indicate high risk in a corresponding category that corresponds to a disease that is particularly severe and/or time sensitive, as the review of these medical scans may be more urgent due to the detection of a more severe and/or time-sensitive condition and/or abnormality. In some cases, the maximum review delay timespan data 5010 can be generated in conjunction with and/or as a function of the triage prioritization scores 4034, where maximum review delay timespan data 5010 is a function of a constant value assigned to a corresponding disease identified as high risk for the patient and/or is further a function of the risk assessment score for the corresponding risk assessment category 4012 as discussed in conjunction with FIGS. 12H and/or 12I.

The maximum review delay timespan data 5010 generated for a particular medical scan and/or particular patient can be added to an urgent scan set 5012 mapping the maximum review delay timespan data 5010 to the medical scan and/or patient. For example, the urgent scan set 5012 maps the maximum review delay timespan data 5010 to medical scan identifier 353 of the corresponding medical scan. The urgent scan set 5012 can alternatively map the maximum review delay timespan data 5010 to any other identifier corresponding to the medical scan or to the patient.

The urgent scan set 5012 be stored locally as illustrated in FIG. 13A. Alternatively, the urgent scan set 5012 can be stored in the medical scan database as scan priority data 427. The maximum review delay timespan data 5010 can otherwise be mapped to the corresponding patient and/or the corresponding medical scan in other memory accessible by the scan review alert system 5002.

Once any of the medical scans pending review have been determined to have been reviewed by a medical professional, they can be removed from this urgent scan set 5012. For example, the scan review alert system 5002 can receive: receive a request to access the medical scan; a notification that the medical scan was retrieved from the medical picture archive system 2620 and/or other embodiment of medical scan database 342; a notification that the medical scan was reviewed by a radiologist; a notification that a medical report corresponding to the medical scan was generated and/or sent to report database 2625; a notification that the patient was subsequently seen by a medical professional and/or received care by a medical professional; and/or another indication that the medical scan was reviewed and/or that a medical professional cared for the patient accordingly.

A review alert generator module 5020 can monitor the urgent scan set 5012 to determine when any maximum review delay timespan data 5010 has elapsed for one or more medical scans still pending review. If maximum review delay timespan data 5010 is determined to have elapsed for a medical scans in the urgent scan set 5012 and if the medical scans is determined to still be pending review, the review alert generator module 5020 can generate and transmit review alert communication data 5030 to one or more client devices 120. For example, the review alert generator module 5020 can compare a current time to the maximum review delay timespan data 5010 of medical scans in the urgent scan set 5012 to determine if any maximum review delay timespan data 5010 has elapsed. As another example, the review alert generator module 5020 automatically schedules sending of review alert communication data 5030 at an end time indicated by the maximum review delay timespan data 5010, and the review alert generator module 5020 automatically sends the review alert communication data 5030 at the scheduled end time based on determining the corresponding medical scan is still pending review at the end time.

In some cases, the maximum review delay timespan data 5010 indicates immediate review of the medical scan and/or indicates no delay before the medical scan be reviewed. In such cases, the maximum review delay timespan data 5010 is optionally not stored and/or the review alert generator module 5020 generates and transmits the review alert communication data 5030 immediately. For example, a medical scan is flagged for this immediate review when the severity score, time sensitivity score, and/or risk factor score compares favorably to an urgent review threshold, and medical scans flagged for immediate review have review alert communication data 5030 generated and transmitted immediately and/or otherwise without any planned and/or scheduled delay of maximum review delay timespan data 5010. In some cases, the maximum review delay timespan data 5010 instead indicates the medical scan requires no urgent review, for example, for medical scans with no detected abnormalities, where no countdown timer is set and/or where an alert is never transmitted for the medical scan.

In some embodiments, a threshold maximum timespan can be instituted based on hospital standards, imaging location standards, and/or other requirements that medical studies be reviewed and/or dictated within this threshold maximum timespan, such as a timespan of 48 hours. To ensure that the medical scan is reviewed within the threshold maximum timespan, the maximum review delay timespan data 5010 can automatically indicate this threshold maximum timespan for medical scans with no detected abnormalities. In some embodiments, the maximum review delay timespan data 5010 can automatically to indicate a buffered-timespan based on the threshold maximum timespan, such as 45 hours to ensure that a radiologist has time to dictate the medical scan within the 48 hour time limit after receiving the alert. In some embodiments, different imaging centers, hospitals, and/or medical entities can have different threshold maximum timespans based on their own requirements. In such cases, different medical scans can automatically be assigned different maximum review delay timespan data 5010 based on being generated at and/or associated with different imaging centers, hospitals, and/or medical entities with different corresponding threshold maximum timespans.

In some cases, the risk assessment category 4012 of one or more risk assessment categories 4012 of FIG. 12F indicate that the corresponding medical scan be flagged for urgent review via communication of review alert communication data 5030. In such cases, a risk assessment score 4006 generated based on abnormality data 442 and/or patient history data 430 that indicates the patient is high risk for a risk assessment category 4012 can cause the corresponding review alert communication data 5030 to be transmitted for the corresponding medical scan immediately and/or after elapsing of a time window indicated by maximum review delay timespan data 5010, based on execution of the high risk protocol instructions 4015 for this risk assessment category 4012 indicating: the corresponding medical scan be included in the urgent scan set 5012, a function to be performed upon the risk assessment score 4006 to calculating the corresponding maximum review delay timespan data 5010, that review alert communication data be transmitted to one or more points of contact immediately and/or after a fixed and/or calculated time window of maximum review delay timespan data 5010 has elapsed.

The review alert communication data 5030 can be sent to one or more medical professionals indicated in medical professional contact data 5022. The medical professional contact data 5022 can be mapped to the medical scan in the urgent scan set 5012 and/or can otherwise be determined based on the medical scan and/or the patient. The medical professional contact data 5022 can indicate names and/or contact information for one or more contacts that should be alerted with the review alert communication data 5030. This can include names and/or contact information associated with a primary physician associated with the given patient; a referring physician associated with the given patient; a radiologist assigned to review the given medical scan; a medical administrator and/or chief technologist assigned to an imaging location, such as an outpatient imaging center, where the medical scan was captured; one or more emergency contacts of the patient; and/or the patient themself. In some cases, the medical professional contact data 5022 is configured for each patient and/or medical scan via user input.

In some cases, the medical professional contact data 5022 is determined automatically based on extracting names and/or contact information from medical scan database 342 from a patient database and/or from another database indicating a list of medical professionals associated with a medical entity corresponding to the medical scan and/or the patient. In some cases, the medical professional contact data 5022 is determined automatically based on extracting names and/or contact information from medical scan database 342 from a patient database and/or from another database indicating a list of medical professionals located within a given radius of patient's address and/or within a given radius of an address of the imaging location that captured the medical scan.

In some cases, the medical professional contact data 5022 can indicate an ordering in the one or more contacts should be notified, for example, where the patient is notified when the primary physician associated with the given patient cannot be reached. For example, a contact in the medical professional contact data 5022 is only notified after attempting to alert all prior contacts in the ordering with no success. In some cases, the alert can include a prompt to acknowledge or respond to the alert, where a next contact in the ordering is transmitted an alert when no acknowledgement or response is received from a previous contact within a threshold period of time. As another example, an administrator or other user can update the medical professional contact data 5022 via user input to an interactive interface to indicate whether or not each contact was sent an alert and/or to indicate whether or not each alerted contact was successfully reached, for example, based on whether or not a telephone call was answered and/or based on another indication of whether the alert was acknowledged.

In some cases, default medical professional contact data 5022 can be utilized for some or all scans indicating one or more radiologists and/or other medical professionals. For example, if the radiologist assigned to review the given medical scan cannot be reached, other backup radiologists will be contacted and/or the medical administrator and/or chief technologist assigned to the corresponding imaging location will be contacted. As another example, scheduling data can be received and/or accessed that indicates vacations and/or work hours of radiologists and/or other medical professionals, where a radiologist is selected for contact based on being automatically determined to be available when the review alert communication data 5030 is determined to be sent based on the scheduling data. As a particular example, the scheduling data indicates the assigned radiologist is out of town for the next week, but the maximum review delay timespan data 5010 indicates the scan should be reviewed within 24 hours. As a result, the review alert generator module 5020 automatically selects a different radiologist for contact based on the scheduling data indicating that this other radiologist is available.

The review alert communication data 5030 can indicate the medical scan identifier of the medical scan and/or can include the image data 410 of the medical scan. The review alert communication data 5030 can indicate a name and/or other patient identifier of the patient associated with the medical scan. The review alert communication data 5030 can indicate some or all of abnormality data and/or can indicate one or more particular diseases and/or medical conditions suspected for the patient based on the abnormality data that warranted the escalation of the medical scan's review. The review alert communication data 5030 can otherwise indicate that the medical scan must be reviewed as soon as possible and/or that the patient should be seen by a medical professional as soon as possible.

The review alert communication data 5030 can include text data, voice data, image data of the medical scan, and/or other types of data conveying some or all of this information. The review alert communication data 5030 can be transmitted via network 150 and/or via a different communication network to one or more client devices 120. The review alert communication data 5030 can be displayed via a client display device 270 of a client device 120 associated with each contact based on the client device 120 receiving the review alert communication data 5030. The review alert communication data 5030 can alternatively or additionally be emitted via speakers of the client device 120 associated with each contact based on the client device 120 receiving the review alert communication data 5030.

In some embodiments, the review alert communication data 5030 is communicated to computer, laptop, and/or cellular device with one or more contacts indicated in the medical professional contact data 5022 via the Internet and/or via another wired and/or wireless communication network. For example, the review alert communication data 5030 is included in the body of an email and is transmitted as an email to one or more email addresses associated with one or more contacts indicated in the medical professional contact data 5022. As another example, the review alert communication data 5030 is included in the body of another message and is transmitted to one or more contacts associated with one or more contacts indicated in the medical professional contact data 5022 in accordance with another messaging platform, such as a secure messaging platform associated with a corresponding medical entity.

In some embodiments, the review alert communication data 5030 is communicated to a telephone and/or cellular device associated with one or more contacts indicated in the medical professional contact data 5022 via a cellular network and/or telephone network. For example, the review alert communication data 5030 can be transmitted as computer-generated voice data and/or computer-generated cellular text data to the telephone and/or cellular device associated with one or more contacts based on corresponding telephone numbers included in the medical professional contact data 5022. As a particular example, the transmitting the review alert communication data 5030 can include autodialing of a telephone number indicated in medical professional contact data 5022 and/or otherwise associated with the one or more contacts and transmitting computer-generated voice data indicating the requirement for the medical scan to be urgently reviewed. In some embodiments, the review alert communication data 5030 is communicated to a telephone and/or cellular device associated with one or more contacts indicated in the medical professional contact data 5022 via a cellular network and/or telephone network. For example, the review alert communication data 5030 can be transmitted as computer-generated voice data and/or computer-generated cellular text data to the telephone and/or cellular device associated with one or more contacts.

In some cases, if a telephone call is not answered and/or a message is not acknowledged as having been read within a predetermined window of time, a next contact in an ordering of the medical professional contact data 5022 is automatically called. This can help ensure the information is conveyed to a medical professional immediately rather than awaiting review in an email inbox during business hours.

In some embodiments, the review alert communication data 5030 indicates medical professional contact data 5022, and is transmitted to a receptionist and/or an employee that is responsible for contacting patients, emergency contacts, primary physicians, radiologists, medical administrators, chief technologists, and/or other contacts via email and/or telephone. The review alert communication data 5030 can be displayed by a display device of the client device 120 to this employee. For example, the names, telephone numbers, email addresses, and/or messaging handles of one or more contacts are displayed to this employee via a display device. This employee can then facilitate the calling, emailing, texting, messaging and/or other communicating with the necessary medical professionals and/or other contacts based on viewing the review alert communication data 5030, which can be preferred in ensuring this sensitive information is conveyed, for example, to a patient and/or emergency contact, via a human voice and/or human-generated message.

FIG. 13B presents a flowchart illustrating a method for execution by a scan review alert system 5002 and/or other subsystem 101 that stores executional instructions that, when executed by at least one processor, cause the system to perform the steps below.

Step 5082 includes receiving a medical scan. Step 5084 includes generating abnormality data for the medical scan by performing an inference function that utilizes a computer vision model. Step 5086 includes selecting a maximum review delay timespan based on the abnormality data. Step 5088 includes facilitating communication of an alert to review the medical scan based on detecting the maximum review delay timespan has elapsed.

It is noted that terminologies as may be used herein such as bit stream, stream, signal sequence, etc. (or their equivalents) have been used interchangeably to describe digital information whose content corresponds to any of a number of desired types (e.g., data, video, speech, text, graphics, audio, etc. any of which may generally be referred to as ‘data’).

As may be used herein, the terms “substantially” and “approximately” provide an industry-accepted tolerance for its corresponding term and/or relativity between items. For some industries, an industry-accepted tolerance is less than one percent and, for other industries, the industry-accepted tolerance is 10 percent or more. Other examples of industry-accepted tolerance range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than +/−1%). Some relativity between items may range from a difference of less than a percentage level to a few percent. Other relativity between items may range from a difference of a few percent to magnitude of differences.

As may also be used herein, the term(s) “configured to”, “operably coupled to”, “coupled to”, and/or “coupling” includes direct coupling between items and/or indirect coupling between items via an intervening item (e.g., an item includes, but is not limited to, a component, an element, a circuit, and/or a module) where, for an example of indirect coupling, the intervening item does not modify the information of a signal but may adjust its current level, voltage level, and/or power level. As may further be used herein, inferred coupling (i.e., where one element is coupled to another element by inference) includes direct and indirect coupling between two items in the same manner as “coupled to”.

As may even further be used herein, the term “configured to”, “operable to”, “coupled to”, or “operably coupled to” indicates that an item includes one or more of power connections, input(s), output(s), etc., to perform, when activated, one or more its corresponding functions and may further include inferred coupling to one or more other items. As may still further be used herein, the term “associated with”, includes direct and/or indirect coupling of separate items and/or one item being embedded within another item.

As may be used herein, the term “compares favorably”, indicates that a comparison between two or more items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1. As may be used herein, the term “compares unfavorably”, indicates that a comparison between two or more items, signals, etc., fails to provide the desired relationship.

As may be used herein, one or more claims may include, in a specific form of this generic form, the phrase “at least one of a, b, and c” or of this generic form “at least one of a, b, or c”, with more or less elements than “a”, “b”, and “c”. In either phrasing, the phrases are to be interpreted identically. In particular, “at least one of a, b, and c” is equivalent to “at least one of a, b, or c” and shall mean a, b, and/or c. As an example, it means: “a” only, “b” only, “c” only, “a” and “b”, “a” and “c”, “b” and “c”, and/or “a”, “b”, and “c”.

As may also be used herein, the terms “processing module”, “processing circuit”, “processor”, “processing circuitry”, and/or “processing unit” may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The processing module, module, processing circuit, processing circuitry, and/or processing unit may be, or further include, memory and/or an integrated memory element, which may be a single memory device, a plurality of memory devices, and/or embedded circuitry of another processing module, module, processing circuit, processing circuitry, and/or processing unit. Such a memory device may be a read-only memory, random access memory, volatile memory, non-volatile memory, static memory, dynamic memory, flash memory, cache memory, and/or any device that stores digital information. Note that if the processing module, module, processing circuit, processing circuitry, and/or processing unit includes more than one processing device, the processing devices may be centrally located (e.g., directly coupled together via a wired and/or wireless bus structure) or may be distributedly located (e.g., cloud computing via indirect coupling via a local area network and/or a wide area network). Further note that if the processing module, module, processing circuit, processing circuitry and/or processing unit implements one or more of its functions via a state machine, analog circuitry, digital circuitry, and/or logic circuitry, the memory and/or memory element storing the corresponding operational instructions may be embedded within, or external to, the circuitry comprising the state machine, analog circuitry, digital circuitry, and/or logic circuitry. Still further note that, the memory element may store, and the processing module, module, processing circuit, processing circuitry and/or processing unit executes, hard coded and/or operational instructions corresponding to at least some of the steps and/or functions illustrated in one or more of the Figures. Such a memory device or memory element can be included in an article of manufacture.

One or more embodiments have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

In addition, a flow diagram may include a “start” and/or “continue” indication. The “start” and “continue” indications reflect that the steps presented can optionally be incorporated in or otherwise used in conjunction with one or more other routines. In addition, a flow diagram may include an “end” and/or “continue” indication. The “end” and/or “continue” indications reflect that the steps presented can end as described and shown or optionally be incorporated in or otherwise used in conjunction with one or more other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

The one or more embodiments are used herein to illustrate one or more aspects, one or more features, one or more concepts, and/or one or more examples. A physical embodiment of an apparatus, an article of manufacture, a machine, and/or of a process may include one or more of the aspects, features, concepts, examples, etc. described with reference to one or more of the embodiments discussed herein. Further, from figure to figure, the embodiments may incorporate the same or similarly named functions, steps, modules, etc. that may use the same or different reference numbers and, as such, the functions, steps, modules, etc. may be the same or similar functions, steps, modules, etc. or different ones.

The term “module” is used in the description of one or more of the embodiments. A module implements one or more functions via a device such as a processor or other processing device or other hardware that may include or operate in association with a memory that stores operational instructions. A module may operate independently and/or in conjunction with software and/or firmware. As also used herein, a module may contain one or more sub-modules, each of which may be one or more modules.

As may further be used herein, a computer readable memory includes one or more memory elements. A memory element may be a separate memory device, multiple memory devices, or a set of memory locations within a memory device. Such a memory device may be a read-only memory, random access memory, volatile memory, non-volatile memory, static memory, dynamic memory, flash memory, cache memory, a quantum register or other quantum memory and/or any other device that stores data in a non-transitory manner. Furthermore, the memory device may be in a form of a solid-state memory, a hard drive memory or other disk storage, cloud memory, thumb drive, server memory, computing device memory, and/or other non-transitory medium for storing data. The storage of data includes temporary storage (i.e., data is lost when power is removed from the memory element) and/or persistent storage (i.e., data is retained when power is removed from the memory element). As used herein, a transitory medium shall mean one or more of: (a) a wired or wireless medium for the transportation of data as a signal from one computing device to another computing device for temporary storage or persistent storage; (b) a wired or wireless medium for the transportation of data as a signal within a computing device from one element of the computing device to another element of the computing device for temporary storage or persistent storage; (c) a wired or wireless medium for the transportation of data as a signal from one computing device to another computing device for processing the data by the other computing device; and (d) a wired or wireless medium for the transportation of data as a signal within a computing device from one element of the computing device to another element of the computing device for processing the data by the other element of the computing device. As may be used herein, a non-transitory computer readable memory is substantially equivalent to a computer readable memory. A non-transitory computer readable memory can also be referred to as a non-transitory computer readable storage medium.

While particular combinations of various functions and features of the one or more embodiments have been expressly described herein, other combinations of these features and functions are likewise possible. The present disclosure is not limited by the particular examples disclosed herein and expressly incorporates these other combinations.

What is claimed is:

1. A risk assessment system, comprising:
a network interface;
a processing system that includes a processor; and
a memory device that stores executable instructions that, when executed by the risk assessment system, configure the processor to perform operations comprising:
receiving, via the network interface, patient history data for a patient;
generating a set of risk assessment scores corresponding to the patient for a set of risk assessment categories based on applying at least one risk assessment function to the patient history data;
identifying one of the set of risk assessment categories as high risk for the patient based on a corresponding one of the set of risk assessment scores;
identifying a high risk protocol corresponding to the one of the set of risk assessment categories; and
facilitating performance of the high risk protocol for the patient based on identification of the one of the set of risk assessment categories as high risk for the patient.

2. The risk assessment system of claim 1, wherein the executable instructions, when executed by the processing system, further configure the processor to perform operations comprising:
comparing each of the set of risk assessment scores to a risk assessment score threshold;
wherein the one of the set of risk assessment categories is identified as high risk based on having a corresponding one of the set of risk assessment scores that compares favorably to a risk assessment score threshold.

3. The risk assessment system of claim 1, wherein the executable instructions, when executed by the processing system, further configure the processor to perform operations comprising:
identifying an ordering of the set of risk assessment categories for the patient based on the set of risk assessment scores;
wherein the one of the set of risk assessment categories is identified as high risk based on being a most-favorably ordered risk assessment category in the ordering of the set of risk assessment categories.

4. The risk assessment system of claim 1, wherein the set of risk assessment categories include a set of disease-based risk assessment categories for each of a set of diseases, and wherein the one of the set of risk assessment categories corresponds to one of the set of diseases.

5. The risk assessment system of claim 4, wherein the executable instructions, when executed by the processing system, further configure the processor to perform operations comprising:
receiving, via the network interface, at least one medical scan of the patient; and
generating abnormality data for the medical scan by performing at least one inference function that utilizes a computer vision model, wherein the computer vision model is trained on a plurality of training medical images;
wherein the set of risk assessment scores corresponding to the patient for the set of disease-based risk assessment categories set is based on applying the at least one risk assessment function to the patient history data and to the abnormality data.

6. The risk assessment system of claim 5, wherein the high risk protocol corresponding to the one of the set of risk assessment categories includes one of a set of possible medical scan image layout protocols based on the one of the set of diseases, and wherein facilitating performance of the high risk protocol for the patient includes facilitating display of the at least one medical scan in accordance with the one of the set of possible medical scan image layout protocols via display device of a client device.

7. The risk assessment system of claim 4, wherein facilitating performance of the high risk protocol includes generating triage data for the patient based on the one of the set of diseases and further includes automatically triaging the patient based on the triage data.

8. The risk assessment system of claim 7, wherein the executable instructions, when executed by the processing system, further configure the processor to perform operations comprising:
receiving, via the network interface, patient history data for a plurality of patients that includes the patient;
generating a plurality of sets of risk assessment scores corresponding to the plurality of patients for the set of disease-based risk assessment categories based on applying the at least one risk assessment function to the patient history data of each of the plurality of patients; and
generating a plurality of triage data for the plurality of patients based on the plurality of sets of risk assessment scores.

9. The risk assessment system of claim 8, wherein the triage data for the patient indicates a first priority, wherein a second patient has triage data in the plurality of triage data indicating a second priority, and wherein the first priority is more favorable than the second priority based on the one of the set of risk assessment scores of the patient for the one of the set of risk assessment categories corresponding to the one of the set of diseases indicating a higher risk than the one of the set of risk assessment scores of the second patient for the one of the set of risk assessment categories corresponding to the one of the set of diseases.

10. The risk assessment system of claim 8, wherein another one of the set of disease-based risk assessment categories corresponding to another one of the set of diseases is identified high risk for a second patient based on one of the plurality of sets of risk assessment scores generated for the second patient, wherein the triage data of the patient indicates a first priority, wherein the second patient has triage data in the plurality of triage data indicating a second priority, and wherein the first priority is more favorable than the second priority based on the one of the set of diseases being one of: more severe than the another one of the set of diseases, or more time-sensitive than the another one of the set of diseases.

11. The risk assessment system of claim 4, wherein the high risk protocol includes one of a set of possible image protocols based on the one of the set of diseases, and wherein facilitating performance of the high risk protocol further includes generating a notification for display via a display device of at least one client device recommending capturing of at least one medical scan of the patient via the one of the set of possible image protocols.

12. The risk assessment system of claim 11, wherein high risk protocol further includes one of a set of possible contrast protocols corresponding to the one of the set of diseases, and wherein facilitating performance of the high risk protocol further includes generating a notification for display via a display device of at least one client device recommending administering contrast to the patient for capturing of the at least one medical scan in accordance with the one of the set of possible contrast protocols.

13. The risk assessment system of claim 12, wherein the executable instructions, when executed by the processing system, further configure the processor to perform operations comprising: receiving, via the network interface, at least one medical scan of the patient, wherein the least one medical scan is captured based on the one of the set of possible image protocols and the one of the set of possible contrast protocols;
generating abnormality data for the at least one medical scan by performing at least one inference function that utilizes a computer vision model, wherein the computer vision model is trained on a plurality of training medical images; and
generating an updated risk assessment score for the one of the set of risk assessment categories based on the abnormality data and based on the patient history data.

14. The risk assessment system of claim 13, wherein the updated risk assessment score for the one of the set of risk assessment categories indicates one of:
a higher risk than the risk assessment score for the one of the set of risk assessment categories based on the abnormality data indicating at least one detected abnormality pattern that corresponds to the one of the set of diseases; or
a lower risk than the risk assessment score for the one of the set of risk assessment categories based on the abnormality data indicating no detected abnormality pattern that corresponds to the one of the set of diseases.

15. The risk assessment system of claim 13, wherein the executable instructions, when executed by the processing system, further configure the processor to perform operations comprising:
generating updated patient history data for the patient based on the abnormality data;
facilitating storage of the updated patient history data; and
retrieving the updated patient history data from storage;
wherein the updated set of risk assessment scores corresponding to the patient is generated based on applying the at least one risk assessment function to the updated patient history data retrieved from storage.

16. The risk assessment system of claim 15, wherein the abnormality data indicates at least one of: fat composition data of anatomical features included in the at least one medical scan, or muscle composition data of anatomical features included in the at least one medical scan, where another one of the set of risk assessment categories corresponds to one of: a stroke category or a joint disorder category, and wherein one of the updated set of risk assessment scores corresponding to the another one of the set of risk assessment categories is generated based on the at least one of: the fat composition data or the muscle composition data;
wherein the executable instructions, when executed by the processing system, further configure the processor to perform operations comprising:
identifying the another one of the set of risk assessment categories as high risk for the patient based on the one of the updated set of risk assessment scores.

17. The risk assessment system of claim 1, wherein the one of the set of risk assessment categories corresponds to an adverse contrast reaction category for at least one of a set of possible contrast protocols, wherein the high risk protocol corresponds to a contrast administering protocol for medical imaging of the patient that indicates not administering contrast in accordance with the at least one of the set of possible contrast protocols.

18. The risk assessment system of claim 17, wherein facilitating performance of the contrast administering protocol includes generating a notification for display via a display device of at least one client device recommending not administering contrast in accordance with the at least one of the set of possible contrast protocols.

19. The risk assessment system of claim 18, wherein the adverse contrast reaction category corresponds to one of: an anaphylactic shock category or an allergic reaction category.

20. A method, comprising:
receiving, via a network interface, patient history data for a patient;
generating a set of risk assessment scores corresponding to the patient for a set of risk assessment categories based on applying at least one risk assessment function to the patient history data;
identifying one of the set of risk assessment categories as high risk for the patient based on a corresponding one of the set of risk assessment scores;
identifying a high risk protocol corresponding to the one of the set of risk assessment categories; and
facilitating performance of the high risk protocol for the patient based on identification of the one of the set of risk assessment categories as high risk for the patient.
